(19) United States
(12) Patent Application Publication
(10) Pub. No.: US 2021/0130745 A1
(43) Pub. Date: May 6, 2021

(54) DETERGENT COMPOSITION

(71) Applicant: The Procter & Gamble Company, Cincinnati, OH (US)

(72) Inventors: Jean-Luc Philippe BETTIOL, Etterbeek (BE); Denis Alfred GONZALES, Brussels (BE); Hilda Andamiche NAMANJA-MAGLIANO, Loveland, OH (US); Juan Esteban VELASQUEZ, Cincinnati, OH (US)

(21) Appl. No.: 17/082,300

(22) Filed: Oct. 28, 2020

(30) Foreign Application Priority Data
Oct. 31, 2019 (EP) 19206504.3
Aug. 17, 2020 (EP) 20191348.0

(51) Int. Cl. C11D 3/386; C11D 11/00; C11D 1/94; C11D 3/39; B08B 3/08

(52) U.S. Cl. CPC C11D 3/38636; C11D 11/0023; C11D 1/29; C11D 3/3942; B08B 3/08; C11D 1/94

(57) ABSTRACT

The need for a hand-dishwashing composition which provides good sudsing and a good suds profile even in the presence of greasy stains comprising higher chain-length saturated and/or unsaturated fatty acid chains, as well as improved removal of such stains, is met by formulating the composition with a P450 fatty acid decarboxylase and a surfactant system.

### REFERENCE TO A SEQUENCE LISTING

This application contains a Sequence Listing in computer readable form. The computer readable form is incorporated herein by reference.

### FIELD OF THE INVENTION

The present invention relates to a hand dishwashing detergent composition comprising a surfactant system and at least one P450 fatty acid decarboxylase. The P450 fatty acid decarboxylases improve sudsing and grease removal by catalyzing the conversion of at least one fatty acid selected from the group consisting of: palmitic acid, stearic acid, oleic acid, linoleic acid, linolenic acid, and mixtures thereof.

### BACKGROUND OF THE INVENTION

Hand-dishwashing detergent compositions should have a good suds profile, in particular a long lasting suds profile. Users typically connate the presence of suds with good residual cleaning, a lack of suds can lead to over-use of the detergent composition, especially in the presence of greasy soils. The appearance of the suds, such as its density and whiteness is also often seen as an indicator of the cleaning efficacy of the wash solution. However, greasy soils inhibit suds generation and promote& suds collapse, even when sufficient surfactancy is present to ensure good cleaning, including grease removal. It has now been found that greasy soils containing higher chain-length saturated and/or unsaturated fatty acid chains are particularly effective at inhibiting sudsing, especially inhibiting long lasting sudsing. In addition, such greasy soils containing higher chain-length saturated and/or unsaturated fatty acid chains are particularly hard to remove from dishes. Such greasy soils comprise long chain fatty acids, especially long chain saturated fatty acids such as palmitic acid and stearic acid, and long chain unsaturated fatty acids, such as oleic acid, linoleic acid, and linolenic acid, which can act as a suds suppressors. Conversion of these long chain saturated and/or unsaturated fatty acids into suds neutral or potentially suds boosting compounds is as such desired.

The use of two different classes fatty acid decarboxylases, OleT-like and UndA-like, to enhance the sudsing profile of detergent compositions have been previously reported (EP 3,243,896B1). However, these enzymes usually have a strong preference for medium chain length fatty acids (e.g. C12, C14), while longer fatty acids (e.g. oleic acid) are converted slowly or not converted. Thus, there remains a need for hand dishwashing compositions that have greater efficacy in transforming long chain fatty acids efficiently into components which are less suds-suppressing.

Moreover, it is often necessary to add more surfactant than necessary for cleaning, in order to provide the desired level of sudsing, as well as meet the users' need for sustained sudsing.

Hence, a need remains for a hand-dishwashing detergent which provides good sudsing and a good suds profile even in the presence of greasy stains comprising higher chain-length saturated and/or unsaturated fatty acid chains, as well as improved removal of such stains. A further need remains for a hand-dishwashing detergent which provides the desired level of sudsing, especially in the presence of such greasy stains, at lower surfactant levels.

EP3556834A relates to a detergent composition, preferably a manual dishwashing detergent composition and method of washing comprising a surfactant system and a fatty acid decarboxylase enzyme. EP3243896A relates to detergent compositions, especially manual dishwashing detergent compositions and method of washing comprising a surfactant system and a fatty acid decarboxylase enzyme. US 2009/0142821 A1 relates to novel variants of cytochrome P450 oxygenases. These variants have an improved ability to use peroxide as an oxygen donor as compared to the corresponding wild-type enzyme. These variants also have an improved thermo-stability as compared to the cytochrome P450 BM-3 F87 A mutant. Preferred variants include cytochrome P450 BM-3 heme domain mutants having I58V, F87A, H100R, F107L, A135S, M145A/V, N239H, S274T, L3241, I366V, K434E, E442K, and/or V446I amino acid substitutions. S CHRISTOPHER DAVIS ET AL, “Oxidation of v-Oxo Fatty Acids by Cytochrome P450 BM-3 (CYP102)”, ARCHIVES OF BIOCHEMISTRY AND BIOPHYSICS, (19960401), vol. 328, no. 1, pages 35-42 discusses the oxidation of aldehydes by cytochrome P450 enzymes either to the corresponding acid or, via a decarboxylation mechanism, to an olefin one carbon shorter than the parent substrate, and explores the factors that control partitioning between these two pathways. The authors have examined the cytochrome P450BM-3 (CYP102)-catalyzed oxidation of fatty acids with a terminal aldehyde group. P450BM-3 has been found to oxidize 18-oxooctadecanoic, 16-oxohexadecanoic, 14-oxotetradecanoic, and 12-oxododecanoic acids exclusively to the corresponding α,ω-diacids. The results demonstrated that aldehyde oxidation by cytochrome P450BM-3 is insensitive to changes in substrate structure expected to stabilize the transition state for decarboxylation. Decarboxylation, in contrast to the oxidation of aldehydes to acids, depends on specific substrate-protein interactions and is enzyme-specific. JAMES BELCHER ET AL., “Structure and Biochemical Properties of the Alkene Producing Cytochrome P450 OleT_{JE} (CYP152L1) from the Jeotgalicoccus sp. 8456 Bacterium”, JOURNAL OF BIOLOGICAL CHEMISTRY, (20140307), vol. 289, no. 10, doi:10.1074/jbc.M113.527325, ISSN 0021-9258, pages 6535-6550, presents the biochemical characterization and crystal structures of a cytochrome P450 fatty acid peroxygenase: the terminal alkene forming OleT_{JE} (CYP152L1) from Jeotgalicoccus sp. 8456. GIRVAN HAZEL M ET AL., “Applications of microbial cytochrome P450 enzymes in biotechnology and synthetic biology”, CURRENT OPINION IN CHEMICAL BIOLOGY, (20160322), vol. 31, doi:10.1016/J.CBPA.2016.02.018, ISSN 1367-5931, pages 136-145, XP029536984 [A] 1-15 is a review focusing on the enzymatic properties and reaction mechanisms of P450 enzymes, and on recent studies that highlight their broad applications in the production of oxychemicals.

### SUMMARY OF THE INVENTION

The present invention relates to a hand-dishwashing composition comprising: a surfactant system comprising at least one anionic surfactant; and a P450 fatty acid decarboxylase.

The present invention further relates to a method of manually washing dishware comprising the steps of delivering a detergent composition according to the invention into a volume of water to form a wash solution and immersing the dishware in the solution.

### DETAILED DESCRIPTION OF THE INVENTION

The need for compositions and methods which provide for good sudsing, including a good long-lasting suds-profile, even in the presence of greasy stains comprising higher chain-length saturated and/or unsaturated fatty acid chains, even at lower surfactant levels, can be met by formulating the hand-dishwashing composition with a P450 fatty acid decarboxylase, wherein said decarboxylase comprises a polypeptide sequence having at least about 80% identity to one or more sequences selected from the group consisting of: SEQ ID NO: 2, 22, 44, 60, 65, 71, 83, 117, 121, 122, 156, and their functional fragments thereof. Such P450 fatty acid decarboxylases catalyse the conversion of at least one fatty acid selected from the group consisting of: palmitic acid, stearic acid, oleic acid, linoleic acid, linolenic acid, and mixtures thereof, preferably stearic acid, oleic acid, and mixtures thereof. Such compositions are also particularly effective at removing grease stains comprising higher chain-length saturated and/or unsaturated fatty acid chains.

### Definitions

As used herein, “dishware” includes cookware and tableware.

As used herein, the articles “a” and “an” when used in a claim, are understood to mean one or more of what is claimed or described.

As used herein, the term “substantially free of” or “substantially free from” means that the indicated material is present in an amount of no more than about 5 wt %, preferably no more than about 2%, and more preferably no more than about 1 wt % by weight of the composition.

As used therein, the term “essentially free of” or “essentially free from” means that the indicated material is present in an amount of no more than about 0.1 wt % by weight of the composition, or preferably not present at an analytically detectible level in such composition. It may include compositions in which the indicated material is present only as an impurity of one or more of the materials deliberately added to such compositions.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

As used herein the phrase “detergent composition” refers to compositions and formulations designed for cleaning soiled surfaces. Such compositions include dish-washing compositions.

As used herein the term “improved suds longevity” means an increase in the duration of visible suds in a washing process cleaning soiled articles using the composition comprising enzymes of use in the compositions of the present invention, compared with the suds longevity provided by the same composition and process in the absence of the enzyme.

As used herein, the term “soiled surfaces” refers to soiled dishware.

As used herein, the term “water hardness” or “hardness” means uncomplexed cation ions (i.e., Ca²⁺ or Mg²⁺) present in water that have the potential to precipitate with anionic surfactants or any other anionically charged detergent actives under alkaline conditions, and thereby diminishing the surfactancy and cleaning capacity of surfactants. Further, the terms “high water hardness” and “elevated water hardness” can be used interchangeably and are relative terms for the purposes of the present invention, and are intended to include, but not limited to, a hardness level containing at least 12 grams of calcium ion per gallon water (gpg, “American grain hardness” units).

As used herein, the terms “protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

As used herein, “polynucleotide” and “nucleic acid” refer to two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine, and cytosine), it may include one or more modified and/or synthetic nucleobases (e.g., inosine, xanthine, hypoxanthine, etc.). Such modified or synthetic nucleobases can be encoding nucleobases.

As used herein, “coding sequence” refers to that portion of a nucleic acid (e.g., a gene) that encodes an amino acid sequence of a protein.

As used herein, “naturally occurring,” “wild-type,” and “WT” refer to the form found in nature. For example, a naturally occurring or wild-type polypeptide or polynucleotide sequence is a sequence present in an organism that can be isolated from a source in nature and which has not been intentionally modified by human manipulation.

As used herein, “non-naturally occurring” or “engineered” or “recombinant” when used in the present invention with reference to (e.g., a cell, nucleic acid, or polypeptide), refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein the term “identity” means the identity between two or more sequences and is expressed in terms of the identity or similarity between the sequences as calculated over the entire length of a sequence aligned against the entire length of the reference sequence. Sequence identity can be measured in terms of percentage identity; the higher the percentage, the more identical the sequences are. The percentage identity is calculated over the length of comparison. For example, the identity is typically calculated over the entire length of a sequence aligned against the entire length of the reference sequence. Methods of alignment of sequences for comparison are well known in the art and identity can be calculated by many known methods. Various programs and alignment algorithms are described in the art. It should be noted that the terms ‘sequence identity’ and ‘sequence similarity’ can be used interchangeably.

As used herein, “percentage of sequence identity,” “percent identity,” and “percent identical” refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity.

As used herein, the term “variant” of P450 fatty acid decarboxylase enzyme means a modified P450 fatty acid decarboxylase enzyme amino acid sequence by or at one or more amino acids (for example 1, 2, 3, 4, 5, 6, 7, 8, 9 or 10 or more amino acid modifications) selected from substitutions, insertions, deletions and combinations thereof. The variant may have “conservative” substitutions, wherein a substituted amino acid has similar structural or chemical properties to the amino acid that replaces it, for example, replacement of leucine with isoleucine. A variant may have “non-conservative” changes, for example, replacement of a glycine with a tryptophan. Variants may also include sequences with amino acid deletions or insertions, or both. Guidance in determining which amino acid residues may be substituted, inserted, or deleted without abolishing the activity of the protein may be found using computer programs well known in the art. Variants may also include truncated forms derived from a wild-type P450 fatty acid decarboxylase enzyme, such as for example, a protein with a truncated N-terminus. Variants may also include forms derived by adding an extra amino acid sequence to a wild-type protein, such as for example, an N-terminal tag, a C-terminal tag or an insertion in the middle of the protein sequence.

As used herein, “reference sequence” refers to a defined sequence to which another sequence is compared. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a comparison window to identify and compare local regions of sequence similarity. The term “reference sequence” is not intended to be limited to wild-type sequences, and can include engineered or altered sequences. For example, a “reference sequence” can be a previously engineered or altered amino acid sequence.

As used herein, “comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered P450 fatty acid decarboxylase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

As used herein, “increased enzymatic activity” and “increased activity” refer to an improved property of a wild-type or an engineered enzyme, which can be represented by an increase in specific activity (e.g., product produced/time/weight protein) or an increase in percent conversion of the substrate to the product (e.g., percent conversion of starting amount of substrate to product in a specified time period using a specified amount of P450 fatty acid decarboxylase) as compared to a reference enzyme. Any property relating to enzyme activity may be affected, including the classical enzyme properties of Km, Vmax or kcat, changes of which can lead to increased enzymatic activity. The P450 fatty acid decarboxylase activity can be measured by any one of standard assays used for measuring P450 fatty acid decarboxylases, such as change in substrate or product concentration. Comparisons of enzyme activities are made using a defined preparation of enzyme, a defined assay under a set condition, and one or more defined substrates, as further described in detail herein. Generally, when enzymes in cell lysates are compared, the numbers of cells and the amount of protein assayed are determined as well as use of identical expression systems and identical host cells to minimize variations in amount of enzyme produced by the host cells and present in the lysates.

As used herein, “conversion” refers to the enzymatic transformation of a substrate to the corresponding product.

As used herein “percent conversion” refers to the percent of the substrate that is converted to the product within a period of time under specified conditions. Thus, for example, the “enzymatic activity” or “activity” of a P450 fatty acid decarboxylase polypeptide can be expressed as “percent conversion” of the substrate to the product.

As used herein, “amino acid difference” or “residue difference” refers to a difference in the amino acid residue at a position of a polypeptide sequence relative to the amino acid residue at a corresponding position in a reference sequence. The positions of amino acid differences generally are referred to herein as “Xn”, where n refers to the corresponding position in the reference sequence upon which the residue difference is based. For example, a “residue difference at position X46 as compared to SEQ ID NO: 1” refers to a difference of the amino acid residue at the polypeptide position corresponding to position 46 of SEQ ID NO:1. Thus, if the reference polypeptide of SEQ ID NO:1 has a tyrosine at position 46, then a “residue difference at position X46 as compared to SEQ ID NO:1” refers to an amino acid substitution of any residue other than tyrosine at the position of the polypeptide corresponding to position 46 of SEQ ID NO:1. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances, the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present invention can include at least one amino acid residue difference relative to a reference sequence, which is indicated by a list of the specified positions where residue differences are present relative to the reference sequence. In embodiments, where more than one amino acid can be used in a specific residue position of a polypeptide, the various amino acid residues that can be used are separated by a “I” (e.g., X46A/G). The present invention includes engineered polypeptide sequences comprising at least one amino acid difference that include either/or both conservative and non-conservative amino acid substitutions. The amino acid sequences of the specific recombinant P450 fatty acid decarboxylase polypeptides included in the Sequence Listing of the present invention include an initiating methionine (M) residue (i.e., M represents residue position 1). The skilled artisan, however, understands that this initiating methionine residue can be removed by biological processing machinery, such as in a host cell or in vitro translation system, to generate a mature protein lacking the initiating methionine residue, but otherwise retaining the enzyme's properties. Consequently, the term “amino acid residue difference relative to SEQ ID NO:1 at position Xn” as used herein may refer to position “Xn” or to the corresponding position (e.g., position (X−1)n) in a reference sequence that has been processed so as to lack the starting methionine.

The term “amino acid substitution set” or “substitution set” refers to a group of amino acid substitutions in a polypeptide sequence, as compared to a reference sequence. A substitution set can have 2, 3, 4, 5, 6, 7, 8, 9, 10, 11, 12, 13, 14, 15, or more amino acid substitutions. In embodiments, a substitution set refers to the set of amino acid substitutions that is present in any of the variant P450 fatty acid decarboxylases.

As used herein, the phrase “conservative amino acid substitutions” refers to the interchangeability of residues having similar side chains, and thus typically involves substitution of the amino acid in the polypeptide with amino acids within the same or similar defined class of amino acids. As such, an amino acid with an aliphatic side chain can be substituted with another aliphatic amino acid (e.g., alanine, valine, leucine, and isoleucine); an amino acid with a hydroxyl side chain can be substituted with another amino acid with a hydroxyl side chain (e.g., serine and threonine); an amino acids having aromatic side chains can be substituted with another amino acid having an aromatic side chain (e.g., phenylalanine, tyrosine, tryptophan, and histidine); an amino acid with a basic side chain can be substituted with another amino acid with a basic side chain (e.g., lysine and arginine); an amino acid with an acidic side chain can be substituted with another amino acid with an acidic side chain (e.g., aspartic acid or glutamic acid); and/or a hydrophobic or hydrophilic amino acid can be replaced with another hydrophobic or hydrophilic amino acid, respectively. The appropriate classification of any amino acid or residue will be apparent to those of skill in the art, especially in light of the detailed invention provided herein.

As used herein, the phrase “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification of the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the polypeptide while retaining enzymatic activity and/or retaining the improved properties of an engineered enzyme. Deletions can be directed to the internal portions and/or terminal portions of the polypeptide. The deletion can comprise a continuous segment or can be discontinuous.

As used herein, “insertion” refers to modification of the polypeptide by addition of one or more amino acids to the reference polypeptide. In embodiments, the improved engineered P450 fatty acid decarboxylase enzymes comprise insertions of one or more amino acids to the naturally occurring P450 fatty acid decarboxylase polypeptide as well as insertions of one or more amino acids to engineered P450 fatty acid decarboxylase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 2, 3, 4, 5, 6, 7, 8, 9, 10, 11, 12, 13, 14, 15, or more amino acid substitutions. A substitution set can refer to the set of amino acid substitutions that is present in any of the variant P450 fatty acid decarboxylases.

As used herein, “fragment” refers to a polypeptide that has an amino-terminal and/or carboxy-terminal deletion, but where the remaining amino acid sequence is identical to the corresponding positions in the sequence. Fragments can typically have about 80%, about 90%, about 95%, about 98%, or about 99% of the full-length P450 fatty acid decarboxylase polypeptide, for example, the polypeptide of SEQ ID NO: 1. In embodiments, the fragment is “biologically active” (i.e., it exhibits the same enzymatic activity as the full-length sequence).

A “functional fragment”, or a “biologically active fragment”, used interchangeably, herein refers to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The improved P450 fatty acid decarboxylase enzymes may be present within a cell, present in the cellular medium, or prepared in various forms, such as lysates or isolated preparations. As such, in embodiments, the wild-type or engineered P450 fatty acid decarboxylase polypeptides of the present invention can be an isolated polypeptide.

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure wild-type or engineered P450 fatty acid decarboxylase polypeptide composition will comprise about 60% or more, about 70% or more, about 80% or more, about 90% or more, about 91% or more, about 92% or more, about 93% or more, about 94% or more, about 95% or more, about 96% or more, about 97% or more, about 98% or more, or about 99% of all macromolecular species by mole or % weight present in the composition. Solvent species, small molecules (<500 Daltons), and elemental ion species are not considered macromolecular species. In embodiments, the isolated improved P450 fatty acid decarboxylase polypeptide is a substantially pure polypeptide composition.

As used herein, when used with reference to a nucleic acid or polypeptide, the term “heterologous” refers to a sequence that is not normally expressed and secreted by an organism (e.g., a wild-type organism). The term can encompass a sequence that comprises two or more subsequences which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature (e.g., a nucleic acid open reading frame (ORF) of the invention operatively linked to a promoter sequence inserted into an expression cassette, such as a vector). “Heterologous polynucleotide” can refer to any polynucleotide that is introduced into a host cell by laboratory techniques, and includes polynucleotides that are removed from a host cell, subjected to laboratory manipulation, and then reintroduced into a host cell.

As used herein, “codon optimized” refers to changes in the codons of the polynucleotide encoding a protein to those preferentially used in a particular organism such that the encoded protein is efficiently expressed in the organism of interest. In embodiments, the polynucleotides encoding the P450 fatty acid decarboxylase enzymes may be codon optimized for optimal production from the host organism selected for expression.

As used herein, “suitable reaction conditions” refer to those conditions in the biocatalytic reaction solution (e.g., ranges of enzyme loading, substrate loading, temperature, pH, buffers, co-solvents, etc.) under which a P450 fatty acid decarboxylase polypeptide of the present invention is capable of converting a substrate compound to a product compound (e.g., conversion of one compound to another compound).

As used herein, “substrate” in the context of a biocatalyst mediated process refers to the compound or molecule acted on by the biocatalyst.

As used herein “product” in the context of a biocatalyst mediated process refers to the compound or molecule resulting from the action of the biocatalyst.

### Detergent Composition

The hand-dishwashing compositions of the present invention formulate a specific surfactant system with a specific P450 fatty acid decarboxylase, in order to provide improved sudsing, especially long-lasting sudsing, in the presence of greasy stains comprising higher chain length saturated and/or unsaturated fatty acids, and improved removal of such stains.

The hand-dishwashing composition is preferably in liquid form, more preferably is an aqueous cleaning composition. As such, the composition can comprise from 50% to 90%, preferably from 60% to 75%, by weight of the total composition of water.

Preferably the pH of the detergent composition of the invention, measured as a 10% product concentration in demineralized water at 20° C., is adjusted to between 3 and 14, more preferably between 4 and 13, more preferably between 6 and 12 and most preferably between 8 and 10. The pH of the detergent composition can be adjusted using pH modifying ingredients known in the art.

The composition of the present invention can be Newtonian or non-Newtonian, preferably Newtonian. Preferably, the composition has a viscosity of from 10 mPa·s to 10,000 mPa·s, preferably from 100 mPa·s to 5,000 mPa·s, more preferably from 300 mPa·s to 2,000 mPa·s, or most preferably from 500 mPa·s to 1,500 mPa·s, alternatively combinations thereof. The viscosity is measured at 20° C. with a Brookfield RT Viscometer using spindle 31 with the RPM of the viscometer adjusted to achieve a torque of between 40% and 60%.

### Surfactant System

The cleaning composition comprises from 5% to 50%, preferably 8% to 45%, more preferably from 15% to 40%, by weight of the total composition of a surfactant system.

For improved sudsing, the surfactant system comprises anionic surfactant. The surfactant system preferably comprises from 60% to 90%, more preferably from 70% to 80% by weight of the surfactant system of the anionic surfactant. Alkyl sulphated anionic surfactants are preferred, particularly those selected from the group consisting of: alkyl sulphate, alkyl alkoxy sulphate, and mixtures thereof. More preferably, the anionic surfactant consists of alkyl sulphated anionic surfactant selected from the group consisting of: alkyl sulphate, alkyl alkoxy sulphate, and mixtures thereof.

For further improvements in sudsing, the surfactant system can comprise less than 30%, preferably less than 15%, more preferably less than 10% of further anionic surfactant, and most preferably the surfactant system comprises no further anionic surfactant. The alkyl sulphated anionic surfactant preferably has an average alkyl chain length of from 8 to 18, preferably from 10 to 14, more preferably from 12 to 14, most preferably from 12 to 13 carbon atoms. The alkyl sulphated anionic surfactant has an average degree of alkoxylation, of less than 5, preferably less than 3, more preferably from 0.5 to 2.0, most preferably from 0.5 to 0.9. Preferably, the alkyl sulphated anionic surfactant is ethoxylated. That is, the alkyl sulphated anionic surfactant has an average degree of ethoxylation, of less than 5, preferably less than 3, more preferably from 0.5 to 2.0, most preferably from 0.5 to 0.9.

The average degree of alkoxylation is the mol average degree of alkoxylation (i.e., mol average alkoxylation degree) of all the alkyl sulphate anionic surfactant. Hence, when calculating the mol average alkoxylation degree, the mols of non-alkoxylated sulphate anionic surfactant are included:

Mol average alkoxylation degree=(x1*alkoxylation degree of surfactant 1+x2*alkoxylation degree of surfactant 2+ . . . )/(x1+x2+ . . . )

wherein x1, x2, . . . are the number of moles of each alkyl (or alkoxy) sulphate anionic surfactant of the mixture and alkoxylation degree is the number of alkoxy groups in each alkyl sulphate anionic surfactant.

The alkyl sulphate anionic surfactant can have a weight average degree of branching of more than 10%, preferably more than 20%, more preferably more than 30%, even more preferably between 30% and 60%, most preferably between 30% and 50%. The alkyl sulphate anionic surfactant can comprise at least 5%, preferably at least 10%, most preferably at least 25%, by weight of the alkyl sulphate anionic surfactant, of branching on the C2 position (as measured counting carbon atoms from the sulphate group for non-alkoxylated alkyl sulphate anionic surfactants, and the counting from the alkoxy-group furthest from the sulphate group for alkoxylated alkyl sulphate anionic surfactants). More preferably, greater than 75%, even more preferably greater than 90%, by weight of the total branched alkyl content consists of C1-C5 alkyl moiety, preferably C1-C2 alkyl moiety. It has been found that formulating the inventive compositions using alkyl sulphate surfactants having the aforementioned degree of branching results in improved low temperature stability. Such compositions require less solvent in order to achieve good physical stability at low temperatures. As such, the compositions can comprise lower levels of organic solvent, of less than 5.0% by weight of the cleaning composition of organic solvent, while still having improved low temperature stability. Higher surfactant branching also provides faster initial suds generation, but typically less suds mileage. The weight average branching, described herein, has been found to provide improved low temperature stability, initial foam generation and suds longevity.

The weight average degree of branching for an anionic surfactant mixture can be calculated using the following formula:

Weight average degree of branching (%)=[(x1*wt % branched alcohol 1 in alcohol 1+x2*wt % branched alcohol 2 in alcohol 2+ . . . )/(x1+x2+ . . . )]*100

wherein x1, x2, . . . are the weight in grams of each alcohol in the total alcohol mixture of the alcohols which were used as starting material before (alkoxylation and) sulphation to produce the alkyl (alkoxy) sulphate anionic surfactant. In the weight average degree of branching calculation, the weight of the alkyl alcohol used to form the alkyl sulphate anionic surfactant which is not branched is included.

The weight average degree of branching and the distribution of branching can typically be obtained from the technical data sheet for the surfactant or constituent alkyl alcohol. Alternatively, the branching can also be determined through analytical methods known in the art, including capillary gas chromatography with flame ionisation detection on medium polar capillary column, using hexane as the solvent. The weight average degree of branching and the distribution of branching is based on the starting alcohol used to produce the alkyl sulphate anionic surfactant.

The alkyl chain of the alkyl sulphated anionic surfactant preferably has a mol fraction of C12 and C13 chains of at least 50%, preferably at least 65%, more preferably at least 80%, most preferably at least 90%. Suds mileage is particularly improved, especially in the presence of greasy soils, when the C13/C12 mol ratio of the alkyl chain is at least 50/50, preferably at least 57/43, preferably from 60/40 to 90/10, more preferably from 60/40 to 80/20, most preferably from 60/40 to 70/30, while not compromising suds mileage in the presence of particulate soils.

Suitable counterions include alkali metal cation earth alkali metal cation, alkanolammonium or ammonium or substituted ammonium, but preferably sodium.

Suitable examples of commercially available alkyl sulphate anionic surfactants include, those derived from alcohols sold under the Neodol® brand-name by Shell, or the Lial®, Isalchem®, and Safol® brand-names by Sasol, or some of the natural alcohols produced by The Procter & Gamble Chemicals company. The alcohols can be blended in order to achieve the desired mol fraction of C12 and C13 chains and the desired C13/C12 ratio, based on the relative fractions of C13 and C12 within the starting alcohols, as obtained from the technical data sheets from the suppliers or from analysis using methods known in the art.

In order to improve surfactant packing after dilution and hence improve suds mileage, the surfactant system preferably comprises a co-surfactant. Preferred co-surfactants are selected from the group consisting of an amphoteric surfactant, a zwitterionic surfactant, and mixtures thereof. The co-surfactant is preferably an amphoteric surfactant, more preferably an amine oxide surfactant. The co-surfactant is included as part of the surfactant system.

The composition preferably comprises from 0.1% to 20%, more preferably from 0.5% to 15% and especially from 2% to 10% by weight of the cleaning composition of the co-surfactant. The surfactant system of the cleaning composition of the present invention preferably comprises from 10% to 40%, preferably from 15% to 35%, more preferably from 20% to 30%, by weight of the surfactant system of a co-surfactant. The anionic surfactant to the co-surfactant weight ratio can be from 1:1 to 8:1, preferably from 2:1 to 5:1, more preferably from 2.5:1 to 4:1.

As mentioned earlier, amine oxide surfactants are preferred for use as a co-surfactant. The amine oxide surfactant can be linear or branched, though linear are preferred. Suitable linear amine oxides are typically water-soluble, and characterized by the formula R1-N(R2)(R3) 0 wherein R1 is a C8-18 alkyl, and the R2 and R3 moieties are selected from the group consisting of C1-3 alkyl groups, C1-3 hydroxyalkyl groups, and mixtures thereof. For instance, R2 and R3 can be selected from the group consisting of: methyl, ethyl, propyl, isopropyl, 2-hydroxethyl, 2-hydroxypropyl and 3-hydroxypropyl, and mixtures thereof, though methyl is preferred for one or both of R2 and R3. The linear amine oxide surfactants in particular may include linear C10-C18 alkyl dimethyl amine oxides and linear C8-C12 alkoxy ethyl dihydroxy ethyl amine oxides.

Preferably, the amine oxide surfactant is selected from the group consisting of: alkyl dimethyl amine oxide, alkyl amido propyl dimethyl amine oxide, and mixtures thereof. Alkyl dimethyl amine oxides are preferred, such as C8-18 alkyl dimethyl amine oxides, or C10-16 alkyl dimethyl amine oxides (such as coco dimethyl amine oxide). Suitable alkyl dimethyl amine oxides include C10 alkyl dimethyl amine oxide surfactant, C10-12 alkyl dimethyl amine oxide surfactant, C12-C14 alkyl dimethyl amine oxide surfactant, and mixtures thereof. C12-C14 alkyl dimethyl amine oxide are particularly preferred.

Alternative suitable amine oxide surfactants include mid-branched amine oxide surfactants. As used herein, “mid-branched” means that the amine oxide has one alkyl moiety having n1 carbon atoms with one alkyl branch on the alkyl moiety having n2 carbon atoms. The alkyl branch is located on the a carbon from the nitrogen on the alkyl moiety. This type of branching for the amine oxide is also known in the art as an internal amine oxide. The total sum of n1 and n2 can be from 10 to 24 carbon atoms, preferably from 12 to 20, and more preferably from 10 to 16. The number of carbon atoms for the one alkyl moiety (n1) is preferably the same or similar to the number of carbon atoms as the one alkyl branch (n2) such that the one alkyl moiety and the one alkyl branch are symmetric. As used herein “symmetric” means that |n1-n2| is less than or equal to 5, preferably 4, most preferably from 0 to 4 carbon atoms in at least 50 wt %, more preferably at least 75 wt % to 100 wt % of the mid-branched amine oxides for use herein. The amine oxide further comprises two moieties, independently selected from a C1-3 alkyl, a C1-3 hydroxyalkyl group, or a polyethylene oxide group containing an average of from about 1 to about 3 ethylene oxide groups. Preferably, the two moieties are selected from a C1-3 alkyl, more preferably both are selected as C1 alkyl.

Alternatively, the amine oxide surfactant can be a mixture of amine oxides comprising a mixture of low-cut amine oxide and mid-cut amine oxide. The amine oxide of the composition of the invention can then comprises:
a) from about 10% to about 45% by weight of the amine oxide of low-cut amine oxide of formula R1R2R3AO wherein R1 and R2 are independently selected from hydrogen, C1-C4 alkyls or mixtures thereof, and R3 is selected from C10 alkyls and mixtures thereof; and
b) from 55% to 90% by weight of the amine oxide of mid-cut amine oxide of formula R4R5R6AO wherein R4 and R5 are independently selected from hydrogen, C1-C4 alkyls or mixtures thereof, and R6 is selected from C12-C16 alkyls or mixtures thereof

In a preferred low-cut amine oxide for use herein R3 is n-decyl, with preferably both R1 and R2 being methyl. In the mid-cut amine oxide of formula R4R5R6A0, R4 and R5 are preferably both methyl.

Preferably, the amine oxide comprises less than about 5%, more preferably less than 3%, by weight of the amine oxide of an amine oxide of formula R7R8R9AO wherein R7 and R8 are selected from hydrogen, C1-C4 alkyls and mixtures thereof and wherein R9 is selected from C8 alkyls and mixtures thereof. Limiting the amount of amine oxides of formula R7R8R9AO improves both physical stability and suds mileage.

Suitable zwitterionic surfactants include betaine surfactants. Such betaine surfactants includes alkyl betaines, alkylamidobetaine, amidazoliniumbetaine, sulphobetaine (INCI Sultaines) as well as the Phosphobetaine, and preferably meets formula (II):

R¹—[CO—X(CH₂)ₙ]ₓ—N⁺(R²)(R₃)—(CH₂)ₘ—[CH(OH)—CH₂]_{y}-Y⁻

wherein in formula (II),

R1 is selected from the group consisting of: a saturated or unsaturated C6-22 alkyl residue, preferably C8-18 alkyl residue, more preferably a saturated C10-16 alkyl residue, most preferably a saturated C12-14 alkyl residue;

X is selected from the group consisting of: NH, NR4 wherein R4 is a C1-4 alkyl residue, O, and S,

n is an integer from 1 to 10, preferably 2 to 5, more preferably 3,

x is 0 or 1, preferably 1,

R2 and R3 are independently selected from the group consisting of: a C1-4 alkyl residue, hydroxy substituted such as a hydroxyethyl, and mixtures thereof, preferably both R2 and R3 are methyl,

m is an integer from 1 to 4, preferably 1, 2 or 3,

y is 0 or 1, and

Y is selected from the group consisting of: COO, SO3, OPO(OR5)O or P(O)(OR5)O, wherein R5 is H or a C1-4 alkyl residue.

Preferred betaines are the alkyl betaines of formula (Ia), the alkyl amido propyl betaine of formula (Ib), the sulphobetaines of formula (Ic) and the amido sulphobetaine of formula (Id):

R¹—N(CH₃)₂—CH₂COO⁻ (IIa)

R¹—CO—NH—(CH₂)₃—N⁺(CH₃)₂—CH₂COO⁻ (IIb)

R¹—N⁺(CH₃)₂—CH₂CH(OH)CH₂SO₃⁻ (IIc)

R¹—CO—NH—(CH₂)₃—N⁺(CH₃)₂—CH₂CH(OH)CH₂SO₃⁻ (IId)

in which R1 has the same meaning as in formula (II). Particularly preferred are the carbobetaines [i.e. wherein Y—═COO— in formula (II)] of formulae (Ia) and (Ib), more preferred are the alkylamidobetaine of formula (Ib).

Suitable betaines can be selected from the group consisting or [designated in accordance with INTO]: capryl/capramidopropyl betaine, cetyl betaine, cetyl amidopropyl betaine, cocamidoethyl betaine, cocamidopropyl betaine, cocobetaines, decyl betaine, decyl amidopropyl betaine, hydrogenated tallow betaine/amidopropyl betaine, isostearamidopropyl betaine, lauramidopropyl betaine, lauryl betaine, myristyl amidopropyl betaine, myristyl betaine, oleamidopropyl betaine, oleyl betaine, palmamidopropyl betaine, palmitamidopropyl betaine, palm-kernelamidopropyl betaine, stearamidopropyl betaine, stearyl betaine, tallowamidopropyl betaine, tallow betaine, undecylenamidopropyl betaine, undecyl betaine, and mixtures thereof. Preferred betaines are selected from the group consisting of: cocamidopropyl betaine, cocobetaines, lauramidopropyl betaine, lauryl betaine, myristyl amidopropyl betaine, myristyl betaine, and mixtures thereof. Cocamidopropyl betaine is particularly preferred.

Preferably, the surfactant system of the composition of the present invention further comprises from 1% to 25%, preferably from 1.25% to 20%, more preferably from 1.5% to 15%, most preferably from 1.5% to 5%, by weight of the surfactant system, of a non-ionic surfactant.

Suitable nonionic surfactants can be selected from the group consisting of: alkoxylated non-ionic surfactant, alkyl polyglucoside (“APG”) surfactant, and mixtures thereof.

Suitable alkoxylated non-ionic surfactants can be linear or branched, primary or secondary alkyl alkoxylated non-ionic surfactants. Alkyl ethoxylated non-ionic surfactant are preferred. The ethoxylated non-ionic surfactant can comprise on average from 9 to 15, preferably from 10 to 14 carbon atoms in its alkyl chain and on average from 5 to 12, preferably from 6 to 10, most preferably from 7 to 8, units of ethylene oxide per mole of alcohol. Such alkyl ethoxylated nonionic surfactants can be derived from synthetic alcohols, such as OXO-alcohols and Fisher Tropsh alcohols, or from naturally derived alcohols, or from mixtures thereof. Suitable examples of commercially available alkyl ethoxylate nonionic surfactants include, those derived from synthetic alcohols sold under the Neodol® brand-name by Shell, or the Lial®, Isalchem®, and Safol® brand-names by Sasol, or some of the natural alcohols produced by The Procter & Gamble Chemicals company.

The compositions of the present invention can comprise alkyl polyglucoside (“APG”) surfactant. The addition of alkyl polyglucoside surfactants have been found to improve sudsing beyond that of comparative nonionic surfactants such as alkyl ethoxylated surfactants. Preferably the alkyl polyglucoside surfactant is a C8-C16 alkyl polyglucoside surfactant, preferably a C8-C14 alkyl polyglucoside surfactant. The alkyl polyglucoside preferably has an average degree of polymerization of between 0.1 and 3, more preferably between 0.5 and 2.5, even more preferably between 1 and 2. Most preferably, the alkyl polyglucoside surfactant has an average alkyl carbon chain length between 10 and 16, preferably between 10 and 14, most preferably between 12 and 14, with an average degree of polymerization of between 0.5 and 2.5 preferably between 1 and 2, most preferably between 1.2 and 1.6. C8-C16 alkyl polyglucosides are commercially available from several suppliers (e.g., Simusol® surfactants from Seppic Corporation; and Glucopon® 600 CSUP, Glucopon® 650 EC, Glucopon® 600 CSUP/MB, and Glucopon® 650 EC/MB, from BASF Corporation).

### P450 Fatty Acid Decarboxylases

P450 fatty acid decarboxylases are enzymes that belong to the cytochrome P450 family CYP152 and catalyze the decarboxylation of fatty acids to alkenes utilizing hydrogen peroxide as co-substrate and heme as a cofactor. The most well studied member of this family is OleTJE (SEQ ID NO: 1), an enzyme endogenous to Jeotgalicoccus sp. 8456 (J. Belcher et al., J. Biol. Chem. (2014), 289, 10: 6535-6550) and is classified as CYP152L1. Variants of OleTJE with fused domains are capable of using molecular oxygen as co-substrate in the presence of an additional co-substrate (Y. Liu et al., Biotechnol. Biofuels (2014) 7: 28). Examples of cytochrome P450 family CYP152 include SEQ ID 1 to 158, some of which are according to the invention and some of which are comparative.

The hand dish-washing compositions of the present invention comprise P450 fatty acid decarboxylases having increased enzymatic activity for long-chain fatty acid substrates, such as oleic acid or linoleic acid, as compared to the well-known naturally occurring wild-type fatty acid decarboxylases reported previously in the art (e.g. OleTJE, SEQ ID NO: 1). Such P450 fatty acid decarboxylases, which may comprise specific amino acid residues at certain positions (e.g. 40, 46, 74, 252, or 317), have been identified as having an increased enzymatic activity towards long chain fatty acids, such as oleic acid or linoleic acid in comparison to the well-known OleTJE (SEQ ID NO: 1) and as such, these decarboxylases provide improved sudsing in the presence of greasy soils comprising such long-chain fatty acids.

The hand dish-washing composition comprises a P450 fatty acid decarboxylase. The decarboxylase comprises a polypeptide sequence having at least 80% identity to one or more sequences selected from the group consisting of: SEQ ID NO: 2, 44, 60, 65, 71, 83, 117, 121, 122, 156, and their functional fragments thereof. The P450 fatty acid decarboxylase can comprise a polypeptide sequence having at least about 85%, 90%, 95%, 98%, 100% identity to one or more sequences selected from the group consisting of: SEQ ID NO: 2, 22, 44, 60, 65, 71, 83, 117, 121, 122, 156, and their functional fragments thereof, preferably SEQ ID NO: 2, 60, 65, 71, 83, 122, and their functional fragments, more preferably SEQ ID NO: 2, 60, 65, 71, 83, 122, and their functional fragments, more most preferably SEQ ID NO: 2, and 122, and most preferably SEQ ID NO: 2.

Said decarboxylase can comprise an amino acid selected from the group consisting of: a) valine, isoleucine, or leucine at position 40, b) alanine, glycine, or valine at position 46, c) valine at position 74, d) lysine at position 252, e) isoleucine, leucine, methionine, or valine at position 317, and combinations thereof; wherein said positions are numbered with reference to SEQ ID NO: 1.

The hand dish-washing composition can comprise a P450 fatty acid decarboxylase comprising an isoleucine, leucine, or valine, at position 40; wherein said positions are numbered with reference to SEQ ID NO: 1. A suitable example of P450 fatty acid decarboxylases comprising an isoleucine at position 40, wherein said position is numbered with reference to SEQ ID NO: 1, is SEQ ID NO: 22. Suitable examples of P450 fatty acid decarboxylases comprising a leucine at position 40, wherein said position is numbered with reference to SEQ ID NO: 1, are SEQ ID NO: 65, and 121. Suitable examples of P450 fatty acid decarboxylases comprising a valine at position 40, wherein said position is numbered with reference to SEQ ID NO: 1, are SEQ ID NO: 2, and 122.

The hand dish-washing composition can comprise a P450 fatty acid decarboxylase comprising an alanine, glycine, valine, or isoleucine at position 46; wherein said positions are numbered with reference to SEQ ID NO: 1. Suitable examples of P450 fatty acid decarboxylases comprising an alanine at position 46, wherein said position is numbered with reference to SEQ ID NO: 1, are SEQ ID NO: 2, and 22. Suitable examples of P450 fatty acid decarboxylases comprising an valine at position 46, wherein said position is numbered with reference to SEQ ID NO: 1, are SEQ ID NO: 44, 60, and 65.

The hand dish-washing composition can comprise a P450 fatty acid decarboxylase comprising a valine at position 74; wherein said positions are numbered with reference to SEQ ID NO: 1. Suitable examples of P450 fatty acid decarboxylases comprising a valine at position 74, wherein said position is numbered with reference to SEQ ID NO: 1, are SEQ ID NO: 2, 71, and 83.

The hand dish-washing composition can comprise a P450 fatty acid decarboxylase comprising a lysine at position 252; wherein said positions are numbered with reference to SEQ ID NO: 1. A suitable example of a P450 fatty acid decarboxylase comprising a lysine at position 252, wherein said position is numbered with reference to SEQ ID NO: 1, is SEQ ID NO: 2.

The hand dish-washing composition can comprise a P450 fatty acid decarboxylase comprising an isoleucine, leucine, methionine, or valine at position 317; wherein said positions are numbered with reference to SEQ ID NO: 1. Suitable examples of P450 fatty acid decarboxylases comprising an isoleucine at position 317, wherein said position is numbered with reference to SEQ ID NO: 1, are SEQ ID NO:22, and 60. Suitable examples of P450 fatty acid decarboxylases comprising a leucine at position 317, wherein said position is numbered with reference to SEQ ID NO: 1, are SEQ ID NO: 2, 65, and 122. A suitable example of P450 fatty acid decarboxylases comprising a methionine at position 317, wherein said position is numbered with reference to SEQ ID NO: 1; is SEQ ID NO: 121. Suitable examples of P450 fatty acid decarboxylases comprising a valine at position 317, wherein said position is numbered with reference to SEQ ID NO: 1; are SEQ ID NO: 44, 71, 83, and 117.

The decarboxylases can have an increased enzymatic activity for oleic acid of at least about 2-fold, 3-fold, 4-fold, 5-fold, 10-fold, 20-fold, 30-fold, 40-fold, 50-fold, 150-fold, 500-fold or more relative to the activity of wild-type decarboxylase (SEQ ID NO: 1) under suitable reaction conditions.

The hand dish-washing composition can comprise a P450 fatty acid decarboxylase having at least about 80%, at least about 90%, at least about 95%, at least about 98%, at least about 100% identity to one or more sequences selected from the group consisting of SEQ ID NO: 2, 22, 44, 60, 65, 71, 83, 117, 121, 122, and 156, and their functional fragments thereof, preferably from the group consisting of SEQ ID NO: 2, 22, 44, 60, 65, 71, 117, 121, and 122. The hand dish-washing composition can comprise a P450 fatty acid decarboxylase having at least about 80%, at least about 90%, at least about 95%, at least about 98%, at least about 100% identity to one or more sequences selected from the group consisting of SEQ ID NO: 2, 22, 151, 156, and their functional fragments. The hand dish-washing composition can comprise a P450 fatty acid decarboxylase having at least about 80%, at least about 90%, at least about 95%, at least about 98%, at least about 100% identity to one or more sequences selected from the group consisting of SEQ ID NO: 2. Suitable examples of P450 fatty acid decarboxylases with at least about 80% identity to SEQ ID NO: 2 are SEQ ID NO: 3, 5, 4, 7, 6, 8, 9, 10, 11, 12, 13, 14, 15, and 151. Suitable examples of P450 fatty acid decarboxylases with at least about 80% identity to SEQ ID NO: 22 are SEQ ID NO: 23, 24, 129. Suitable examples of P450 fatty acid decarboxylases with at least about 80% identity to SEQ ID NO: 44 are SEQ ID NO: 39, 40, 41, 43, 44, 45, 46, 47, 48, 49, 50, 85, 126, 127, 128, 130, 131, 132, 133, 134, 135, 136, 137, 138, 139, 140, 141, 142, 143, 144, 145, and 146. Suitable examples of P450 fatty acid decarboxylases with at least about 80% identity to SEQ ID NO: 60 are SEQ ID NO: 54, 55, 56, 57, 58, 59, 61, 62, and 63. A suitable example of P450 fatty acid decarboxylases with at least about 80% identity to SEQ ID NO: 65 is SEQ ID NO: 64. Suitable examples of P450 fatty acid decarboxylases with at least about 80% identity to SEQ ID NO: 71 are SEQ ID NO: 67, 68, 69, 70, 72, 73, 74, 75, 76, 77, 78, 79, 80, 81, 123, 124, and 125. Suitable examples of P450 fatty acid decarboxylases with at least about 80% identity to SEQ ID NO: 117 are SEQ ID NO: 114, 115, 116, 118, and 119.

Identity, or homology, percentages as mentioned herein in respect of the present invention are those that can be calculated, for example, with AlignX obtainable from Thermo Fischer Scientific or with the alignment tool from Uniprot (https://www.uniprot.org/align/). Alternatively, a manual alignment can be performed. For enzyme sequence comparison the following settings can be used: Alignment algorithm: Needleman and Wunsch, J. Mol. Biol. 1970, 48: 443-453. As a comparison matrix for amino acid similarity the Blosum62 matrix is used (Henikoff S. and Henikoff J. G., P.N.A.S. USA 1992, 89: 10915-10919). The following gap scoring parameters are used: Gap penalty: 12, gap length penalty: 2, no penalty for end gaps.

A given sequence is typically compared against the full-length sequence or fragments of SEQ ID NO: 1, 2, 22, 44, 60, 65, 71, 83, 117, 121, 122, or 156 to obtain a score. Polypeptides of the present disclosure can include polypeptides containing an amino acid sequence having at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or 100% identity to the amino acid sequence of any one of SEQ ID NO: 2, 22, 44, 60, 65, 71, 83, 117, 121, 122, and 156, preferably to the amino acid sequence of any one of SEQ ID NO: 2, 22, 44, 60, 65, 71, 83, 117, 121, 122, and 156. Polypeptides of the disclosure can also include polypeptides having at least about 10, at least about 12, at least about 14, at least about 16, at least about 18, at least about 20, at least about 30, at least about 40, at least about 50, at least about 60, at least about 70, or at least about 80 consecutive amino acids of the amino acid sequence of any one of SEQ ID NO: 2, 22, 44, 60, 65, 71, 83, 117, 121, 122, and 156, preferably of the amino acid sequence of any one of SEQ ID NO: 2, 22, 44, 60, 65, 71, 83, 117, 121, and 122.

The hand dish-washing composition can comprise variants of P450 fatty acid decarboxylases, as previously described. Variants of P450 fatty acid decarboxylases include polypeptide sequences resulting from modification of a wild-type P450 fatty acid decarboxylase at one or more amino acids. A variant includes a “modified enzyme” or a “mutant enzyme” which encompasses proteins having at least one substitution, insertion, and/or deletion of an amino acid. A modified enzyme may have 1, 2, 3, 4, 5, 6, 7, 8, 9 or 10 or more amino acid modifications (selected from substitutions, insertions, deletions and combinations thereof).

The variants may have “conservative” substitutions. Suitable conservative substitutions include one conservative substitution in the enzyme, such as a conservative substitution in SEQ ID NO: 2, 22, 44, 60, 65, 71, 83, 117, 121, 122, 156, and their functional fragments thereof, preferably a conservative substitution in SEQ ID NO: 2, 22, 44, 60, 65, 71, 83, 117, 121, 122, and their functional fragments thereof. Other suitable substitutions include 10 or fewer conservative substitutions in the protein, such as five or fewer. An enzyme of use in the invention may therefore include 1, 2, 3, 4, 5, 6, 7, 8, 9, 10 or more conservative substitutions. An enzyme can be produced to contain one or more conservative substitutions by manipulating the nucleotide sequence that encodes that enzyme using, for example, standard procedures such as site-directed mutagenesis or PCR. Examples of amino acids which may be substituted for an original amino acid in an enzyme and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

The hand dish-washing composition can comprise variants of the P450 fatty acid decarboxylase which comprise a polypeptide sequence comprising at least one amino acid substitution at positions selected from the group consisting of: 40, 46, 74, 79, and combinations thereof; wherein said positions are numbered with reference to SEQ ID NO: 1.

The variant of the P450 fatty acid decarboxylase can comprise a polypeptide sequence comprising at least one amino acid substitution selected from the group consisting of: F79A, R245P, P246R, K252Q, F253A, F256A, R286Q, and combinations thereof; wherein said positions are numbered with reference to SEQ ID NO: 1.

The variant of the P450 fatty acid decarboxylase can comprise a polypeptide sequence comprising at least one amino acid substitution at positions selected from the group consisting of: 77, 78, 79, 85, 166, 169, 170, 190, 193, 238, 240, 241, 242, 243, 244, 245, 246, 247, 248, 249, and combinations thereof; wherein said positions are numbered with reference to SEQ ID NO: 1; and wherein said amino acid substitution is for an amino acid selected from the group consisting of aspartate and glutamate.

The variant of the P450 fatty acid decarboxylase can comprise a polypeptide sequence comprising at least one amino acid substitution selected from the group consisting of: F80A, R246P, P247R, K253Q, F254A, F257A, R288Q, and combinations thereof; wherein said positions are numbered with reference to SEQ ID NO: 2.

It is important that variants of enzymes retain and preferably improve the ability of the wild-type protein to catalyze the conversion of the fatty acids. Some performance drop in a given property of variants may of course be tolerated, but the variants should retain and preferably improve suitable properties for the relevant application for which they are intended. Screening of variants of one of the wild-types can be used to identify whether they retain and preferably improve appropriate properties.

The decarboxylase polypeptides described herein are not restricted to the genetically encoded amino acids. Thus, in addition to the genetically encoded amino acids, the polypeptides described herein may be comprised, either in whole or in part, of naturally-occurring and/or synthetic non-encoded amino acids. Certain commonly encountered non-encoded amino acids of which the polypeptides described herein may be comprised include, but are not limited to: the D-stereoisomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (Nal); 2-chlorophenylalanine (Oct); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opcf); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenylpentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutamic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aOly); propargylglycine (pgGly); homoalanine (hAla); norvaline (nVal); homoleucine (hLeu), homovaline (hVal); homoisoleucine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised will be apparent to those of skill in the art. These amino acids may be in either the L- or D-configuration.

Suitable variants in the form of truncated forms or fragments can be derived from a wild-type enzyme, such as a protein with a truncated N-terminus or a truncated C-terminus. Suitable variants of decarboxylase enzymes can comprise a fragment of any of the decarboxylase polypeptides described herein that retain the functional decarboxylase activity and/or an improved property of an engineered decarboxylase polypeptide. Accordingly, the hand dish-washing composition can comprise a polypeptide fragment having decarboxylase activity (e.g., capable of converting substrate to product under suitable reaction conditions), wherein the fragment comprises at least about 80%, 90%, 95%, 98%, or 99% of a full-length amino acid sequence of an engineered polypeptide of use in the present invention.

Suitable decarboxylase enzymes can have an amino acid sequence comprising an insertion as compared to any one of the decarboxylase polypeptide sequences described herein. Thus, the insertions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, where the associated functional activity and/or improved properties of the decarboxylase described herein is maintained. The insertions can be to amino or carboxy terminus, or internal portions of the decarboxylase polypeptide. The variants can be derived by adding an extra amino acid sequence, such as an N-terminal tag or a C-terminal tag. Non-limiting examples of tags are maltose binding protein (MBP) tag, glutathione S-transferase (GST) tag, thioredoxin (Trx) tag, His-tag, and any other tags known by those skilled in art. Tags can be used to improve solubility and expression levels during fermentation or as a handle for enzyme purification.

Enzymes can also be modified by a variety of chemical techniques to produce derivatives having essentially the same or preferably improved activity as the unmodified enzymes, and optionally having other desirable properties. For example, carboxylic acid groups of the protein, whether carboxyl-terminal or side chain, may be provided in the form of a salt of a pharmaceutically-acceptable cation or esterified, for example to form a C1-C6 alkyl ester, or converted to an amide, for example of formula CONR1R2 wherein R1 and R2 are each independently H or C1-C6 alkyl, or combined to form a heterocyclic ring, such as a 5- or 6-membered ring. Amino groups of the enzyme, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C1-C20 alkyl or dialkyl amino or further converted to an amide. Hydroxyl groups of the protein side chains may be converted to alkoxy or ester groups, for example C1-C20 alkoxy or C1-C20 alkyl ester, using well-recognized techniques. Phenyl and phenolic rings of the protein side chains may be substituted with one or more halogen atoms, such as F, C1, Br or I, or with C1-C20 alkyl, C1-C20 alkoxy, carboxylic acids and esters thereof, or amides of such carboxylic acids. Methylene groups of the protein side chains can be extended to homologous C2-C4 alkylenes. Thiols can be protected with any one of a number of well-recognized protecting groups, such as acetamide groups. Those skilled in the art will also recognize methods for introducing cyclic structures into the proteins of this disclosure to select and provide conformational constraints to the structure that result in enhanced stability.

The enzymes can be provided on a solid support, such as a membrane, resin, solid carrier, or other solid phase material. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location.

The polypeptides having decarboxylase activity can be bound or immobilized on the solid support such that they retain at least a portion of their improved properties relative to a reference polypeptide (e.g., SEQ ID NO: 1). Accordingly, it is further contemplated that suitable methods of using the decarboxylase polypeptides can be carried out using the same decarboxylase polypeptides bound or immobilized on a solid support.

The decarboxylase polypeptide can be bound non-covalently or covalently. Various methods for conjugation and immobilization of enzymes to solid supports (e.g., resins, membranes, beads, glass, etc.) are well known in the art. Other methods for conjugation and immobilization of enzymes to solid supports (e.g., resins, membranes, beads, glass, etc.) are well known in the art (See, e.g., Yi et al., Proc. Biochem., 42: 895-898 [2007]; Martin et al., Appl. Microbiol. Biotechnol., 76: 843-851 [2007]; Koszelewski et al. J. Mol. Cat. B: Enz., 63: 39-44 [2010]; Truppo et al., Org. Proc. Res. Develop., published online: dx.doi.org/10.1021/op200157c; and Mateo et al., Biotechnol. Prog., 18:629-34 [2002], etc.). Solid supports useful for immobilizing the decarboxylase polypeptides of use in the present invention include, but are not limited to, beads or resins comprising polymethacrylate with epoxide functional groups, polymethacrylate with amino epoxide functional groups, styrene/DVB copolymer or polymethacrylate with octadecyl functional groups.

The enzymes may be incorporated into the hand dish-washing compositions via an additive particle, such as an enzyme granule or in the form of an encapsulate or may be added in the form of a liquid formulation. Encapsulating the enzymes promote the stability of the enzymes in the composition and helps to counteract the effect of any hostile compounds present in the composition, such as bleach, protease, surfactant, chelant, etc. The P450 fatty acid decarboxylase enzymes may be the only enzymes in the additive particle or may be present in the additive particle in combination with one or more additional co-enzymes.

The hand dish-washing composition can comprise a P450 fatty acid decarboxylase, wherein said P450 fatty acid decarboxylase is present in an amount of from 0.0001 wt % to 1 wt %, preferably from 0.001 wt % to 0.2 wt %, by weight of the consumer product composition, based on active protein.

The hand dish-washing composition can comprise one or more co-enzymes selected from the group consisting of: fatty-acid peroxidases (EC 1.11.1.3), unspecific peroxygenases (EC 1.11.2.1), plant seed peroxygenases (EC 1.11.2.3), fatty acid peroxygenases (EC1.11.2.4), linoleate diol synthases (EC 1.13.11.44), 5,8-linoleate diol synthases (EC 1.13.11.60 and EC 5.4.4.5), 7,8-linoleate diol synthases (EC 1.13.11.60 and EC 5.4.4.6), 9,14-linoleate diol synthases (EC 1.13.11.B1), 8,11-linoleate diol synthases, oleate diol synthases, other linoleate diol synthases, unspecific monooxygenase (EC 1.14.14.1), alkane 1-monooxygenase (EC 1.14.15.3), oleate 12-hydroxylases (EC 1.14.18.4), fatty acid amide hydrolases (EC 3.5.1.99), fatty acid photodecarboxylases (EC 4.1.1.106), oleate hydratases (EC 4.2.1.53), linoleate isomerases (EC 5.2.1.5), linoleate (10E,12Z)-isomerases (EC 5.3.3.B2), non-heme fatty acid decarboxylases (UndA-like), alpha-dioxygenases, amylases, lipases, proteases, cellulases, and mixtures thereof; preferably fatty-acid peroxidases (EC 1.11.1.3), unspecific peroxygenases (EC 1.11.2.1), plant seed peroxygenases (EC 1.11.2.3), and fatty acid peroxygenases (EC1.11.2.4), non-heme fatty acid decarboxylases (UndA-like), alpha-dioxygenases, and mixtures thereof.

Where necessary, the composition comprises, provides access to, or forms in situ any additional substrate necessary for the effective functioning of the enzyme. For example, molecular hydrogen peroxide can be provided as an additional substrate for P450 fatty acid decarboxylases. In embodiments, the consumer product composition may be supplemented with heme and/or a source of iron to enhance or facilitate the conversion of the fatty acids.

The P450 fatty acid decarboxylase can comprise a heme cofactor selected from the group comprising: heme a, heme b, heme c, heme d, heme i, heme m, heme o, heme s, their derivatives, and mixtures thereof; preferably heme b. The heme cofactor can be covalently attached to the P450 fatty acid decarboxylases.

The P450 fatty acid decarboxylase can comprise a heme cofactor comprising: a) a porphyrin group and b) a metal. Non-limiting examples of porphyrin groups are: protoporphyrin IX, N-methyl protoporphyrin IX, protoporphyrin IX monomethyl ester, protoporphyrin IX dimethyl ester, protoporphyrin IX diamide, protoporphyrin IX bis thiosulfate, porphin, phthalocyanine, octaethylporphoyrin, tetraphenylporphyrin, and their derivatives; preferably protoporphyrin IX. Non-limiting examples of metals are: Mg, Al, V, Cr, Mn, Fe, Co, Ni, Cu, Zn, Ga, Ge, Mo, Tc, Ru, Rh, Pd, Ag, Cd, In, Sn, Sb, W, Re, Os, Ir, Pt, Au, Hg, Tl, Pb, Bi, and mixtures thereof; preferably Fe. The P450 fatty acid decarboxylase can comprise a heme cofactor comprising a cation selected from the group comprising: Mg²⁺, Cr³⁺, Mn³⁺, Fe³⁺, Co³⁺, Ni²⁺, Cu²⁺, Zn²⁺, Ga³⁺, Rh²⁺, Pd²⁺, Ag²⁺, In³⁺, Sn⁴⁺, VO²⁺, and mixtures thereof; preferably Fe³⁺. The P450 fatty acid decarboxylase can comprise a heme cofactor comprising an axially bound ligand. Suitable ligands include: chloride, methyl group, carbonyl group, hydroxide group, and tetrahydrofuran.

Where necessary, the composition comprises, provides access to or forms in situ any additional substrate necessary for the effective functioning of the enzyme. For example, when molecular oxygen is an additional substrate, it can be obtained from the atmosphere or from a precursor that can be transformed to produce oxygen in situ. In many applications, oxygen from the atmosphere can be present in sufficient amounts.

### Methods of Producing P450 Fatty Acid Decarboxylase Polypeptides

Standard methods of culturing organisms such as, for example, bacteria and yeast, for production of enzymes are well-known in the art and are described herein. For example, host cells may be cultured in a standard growth media under standard temperature and pressure conditions, and in an aerobic environment. Standard growth media for various host cells are commercially available and well-known in the art, as are standard conditions for growing various host cells.

The P450 fatty acid decarboxylase enzymes expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B (Sigma-Aldrich). Chromatographic techniques for isolation of the P450 fatty acid decarboxylase polypeptide include, among others, reverse phase chromatography high performance liquid chromatography (HPLC), ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

The P450 fatty acid decarboxylases may also be prepared and used in the form of cells expressing the enzymes, as crude extracts, or as isolated or purified preparations. The P450 fatty acid decarboxylases may be prepared as lyophilizates, in powder form (e.g., acetone powders), or prepared as enzyme solutions. The P450 fatty acid decarboxylases can be in the form of substantially pure preparations.

### Adjunct Ingredients

The cleaning composition herein may optionally comprise a number of other adjunct ingredients such as additional enzymes, enzyme stabilisers, antioxidant compounds, free radical scavengers, source of hydrogen peroxide, organic solvents, polymers, cleaning amines, chelants, builders (e.g., preferably citrate), structurants, emollients, humectants, skin rejuvenating actives, scrubbing particles, bleach and bleach activators, perfumes, malodor control agents, pigments, dyes, opacifiers, beads, pearlescent particles, capsules, inorganic cations such as alkaline earth metals such as Ca/Mg-ions, antibacterial agents, preservatives, viscosity adjusters (e.g., salt such as NaCl, and other mono-, di- and trivalent salts) and pH adjusters and buffering means (e.g., carboxylic acids such as citric acid, HCl, NaOH, KOH, alkanolamines, phosphoric and sulfonic acids, carbonates such as sodium carbonates, bicarbonates, sesquicarbonates, borates, silicates, phosphates, imidazole and alike).

### Additional Enzymes

Preferred compositions of the invention comprise one or more enzymes selected from lipases, proteases, cellulases, amylases, and any combination thereof.

Each additional enzyme is typically present in an amount from 0.0001 wt % to 1 wt % (weight of active protein) more preferably from 0.0005 wt % to 0.5 wt %, most preferably 0.005-0.1% by weight of the detergent composition. It may be particularly preferred for the compositions of the present invention to additionally comprise a lipase enzyme. Lipases break down fatty ester soils into fatty acids which are then acted upon by the saturated and/or unsaturated fatty acid-transforming enzyme according to the invention into suds neutral or suds boosting agents.

It may be particularly preferred for the compositions of the present invention to additionally comprise a protease enzyme. Since oleic acid and other foam suppressing saturated and/or unsaturated fatty acids are present in body soils or even human skin, as protease enzyme acts as a skin care agent, or breaks down proteinaceous soils, fatty acids released are broken down, preventing suds suppression.

It may be particularly preferred for the compositions of the present invention to additionally comprise an amylase enzyme. Since oily soils are commonly entrapped in starchy soils, the amylase and saturated and/or unsaturated fatty acid transforming enzymes work synergistically together: fatty acid soils are released by breakdown of starchy soils with amylase, thus, the saturated and/or unsaturated fatty acid transforming enzyme according to the invention is particularly effective in ensuring there is no negative impact on suds in the wash liquor.

### Enzyme Stabiliser

Preferably the composition of the invention comprises an enzyme stabilizer. Suitable enzyme stabilizers may be selected from the group consisting of (a) univalent, bivalent and/or trivalent cations preferably selected from the group of inorganic or organic salts of alkaline earth metals, alkali metals, aluminum, iron, copper and zinc, preferably alkali metals and alkaline earth metals, preferably alkali metal and alkaline earth metal salts with halides, sulfates, sulfites, carbonates, hydrogencarbonates, nitrates, nitrites, phosphates, formates, acetates, propionates, citrates, maleates, tartrates, succinates, oxalates, lactates, and mixtures thereof. The salt can be selected from the group consisting of sodium chloride, calcium chloride, potassium chloride, sodium sulfate, potassium sulfate, sodium acetate, potassium acetate, sodium formate, potassium formate, calcium lactate, calcium nitrate and mixtures thereof. Most preferred are salts selected from the group consisting of calcium chloride, potassium chloride, potassium sulfate, sodium acetate, potassium acetate, sodium formate, potassium formate, calcium lactate, calcium nitrate, and mixtures thereof, and in particular potassium salts selected from the group of potassium chloride, potassium sulfate, potassium acetate, potassium formate, potassium propionate, potassium lactate and mixtures thereof. Most preferred are potassium acetate and potassium chloride. Preferred calcium salts are calcium formate, calcium lactate and calcium nitrate including calcium nitrate tetrahydrate. Calcium and sodium formate salts may be preferred. These cations are present at at least 0.01 wt %, preferably at least 0.03 wt %, more preferably at least 0.05 wt %, most preferably at least 0.25 wt % up to 2 wt % or even up to 1 wt % by weight of the total composition. These salts are formulated from 0.1 wt % to 5 wt %, preferably from 0.2 wt % to 4 wt %, more preferably from 0.3 wt % to 3 wt %, most preferably from 0.5 wt % to 2 wt % relative to the total weight of the composition. Further enzyme stabilizers can be selected from the group (b) carbohydrates selected from the group consisting of oligosaccharides, polysaccharides and mixtures thereof, such as a monosaccharide glycerate as described in WO201219844; (c) mass efficient reversible protease inhibitors selected from the group consisting of phenyl boronic acid and derivatives thereof, preferably 4-formyl phenylboronic acid; (d) alcohols such as 1,2-propane diol, propylene glycol; (e) peptide aldehyde stabilizers such as tripeptide aldehydes such as Cbz-Gly-Ala-Tyr-H, or disubstituted alaninamide; (f) carboxylic acids such as phenyl alkyl dicarboxylic acid as described in WO2012/19849 or multiply substituted benzyl carboxylic acid comprising a carboxyl group on at least two carbon atoms of the benzyl radical such as described in WO2012/19848, phthaloyl glutamine acid, phthaloyl asparagine acid, aminophthalic acid and/or an oligoamino-biphenyl-oligocarboxylic acid; and (g) mixtures thereof.

The composition of the present invention may optionally comprise from 0.01% to 3%, preferably from 0.05% to 2%, more preferably from 0.2% to 1.5%, or most preferably 0.5% to 1%, by weight of the total composition of a salt, preferably a monovalent, divalent inorganic salt or a mixture thereof, preferably sodium chloride. Most preferably the composition alternatively or further comprises a multivalent metal cation in the amount of from 0.01 wt % to 3 wt %, preferably from 0.05% to 2%, more preferably from 0.2% to 1.5%, or most preferably 0.5% to 1% by weight of said composition, preferably said multivalent metal cation is magnesium, aluminium, copper, calcium or iron, more preferably magnesium, most preferably said multivalent salt is magnesium chloride. Without wishing to be bound by theory, it is believed that use of a multivalent cation helps with the formation of protein/protein, surfactant/surfactant or hybrid protein/surfactant network at the oil water and air water interface that is strengthening the suds.

Preferably the composition of the present invention comprises one or more carbohydrates selected from the group comprising O-glycan, N-glycan, and mixtures thereof. Preferably the cleaning composition further comprises one or more carbohydrates selected from the group comprising derivatives of glucose, mannose, lactose, galactose, allose, altrose, gulose, idose, talose, fucose, fructose, sorbose, tagatose, psicose, arabinose, ribose, xylose, lyxose, ribulose, and xylulose. More preferably the cleaning composition comprises one or more carbohydrates selected from the group of α-glucans and β-glucans. Glucans are polysaccharides of D-glucose monomers, linked by glycosidic bonds. Suitable α-glucans are dextran, starch, floridean starch, glycogen, pullulan, and their derivatives. Suitable β-glucans are cellulose, chrysolaminarin, curdlan, laminarin, lentinan, lichenin, oat beta-glucan, pleuran, zymosan, and their derivatives.

### Hydrotrope

The composition of the present invention may optionally comprise from 1% to 10%, or preferably from 0.5% to 10%, more preferably from 1% to 6%, or most preferably from 0.1% to 3%, or combinations thereof, by weight of the total composition of a hydrotrope, preferably sodium cumene sulfonate. Other suitable hydrotropes for use herein include anionic-type hydrotropes, particularly sodium, potassium, and ammonium xylene sulfonate, sodium, potassium and ammonium toluene sulfonate, sodium potassium and ammonium cumene sulfonate, and mixtures thereof, as disclosed in U.S. Pat. No. 3,915,903. Preferably the composition of the present invention is isotropic. An isotropic composition is distinguished from oil-in-water emulsions and lamellar phase compositions. Polarized light microscopy can assess whether the composition is isotropic. See e.g., The Aqueous Phase Behaviour of Surfactants, Robert Laughlin, Academic Press, 1994, pp. 538-542. Preferably an isotropic composition is provided. Preferably the composition comprises 0.1% to 3% by weight of the total composition of a hydrotrope, preferably wherein the hydrotrope is selected from sodium, potassium, and ammonium xylene sulfonate, sodium, potassium and ammonium toluene sulfonate, sodium potassium and ammonium cumene sulfonate, and mixtures thereof.

### Antioxidant Compounds and Free Radical Scavengers

Antioxidant compounds and free radical scavengers can generally protect enzyme from degradation by preventing excessive generation of singlet oxygen and peroxy radicals that promote alteration of enzyme structure leading to short TON of Enzymes. Not to be limited by theory, a general discussion of the mode of action for antioxidants and free radical scavengers is disclosed in Kirk Othmer, The Encyclopedia of Chemical Technology, Volume 3, pages 128-148, Third Edition (1978).

The composition may optionally contain an anti-oxidant present from about 0.001% to about 2.0% by weight of the detergent composition. The antioxidant can be present at a concentration in the range 0.01 to 0.1% by weight of the detergent composition. Mixtures of anti-oxidants may be used and in some embodiments, may be preferred.

One class of anti-oxidants used in the present invention is alkylated phenols, having the general formula:

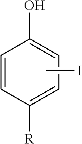

wherein R is C1-C22 linear or branched alkyl, preferably methyl or branched C3-C6 alkyl, Ci-C₆ alkoxy, preferably methoxy, or CH₂CH₂C(O)OR′, wherein R′ is H, a charge balancing counterion or C1-C22 linear or branched alkyl; Ri is a C3-C6 branched alkyl, preferably tert-butyl; x is 1 or 2. Hindered phenolic compounds are a preferred type of alkylated phenols having this formula. A preferred hindered phenolic compound of this type is 3,5-di-tert-butyl-4-hydroxytoluene (BHT).

Furthermore, the anti-oxidant used in the composition may be selected from the group consisting of a-, b-, g-, d-tocopherol, ethoxyquin, 2, 2 4-trimethyl-1,2-dihydroquinoline, 2,6-di-tert-butyl hydroquinone, tert-butyl hydroxyanisole, lignosulphonic acid and salts thereof, and mixtures thereof. It is noted that ethoxyquin (1,2-dihydro-6-ethoxy-2,2,4-trimethylquinoline) is marketed under the name Raluquin™ by the company Raschig™. Other types of anti-oxidants that may be used in the composition are 6-hydroxy-2, 5,7,8-tetramethylchroman-2-carboxylic acid (Trolox™) and 1,2-benzisothiazoline-3-one (Proxel GXL™).

A further class of anti-oxidants which may be suitable for use in the composition is a benzofuran or benzopyran derivative having the formula:

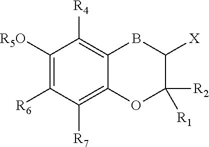

wherein Ri and R₂ are each independently alkyl or Ri and R₂ can be taken together to form a C₅-C₆ cyclic hydrocarbyl moiety; B is absent or CH₂; R₄ is Ci-Ce alkyl; R₅ is hydrogen or —C(0)R₃ wherein R₃ is hydrogen or C₁-C₁₉ alkyl; Re is Ci-Ce alkyl; R₇ is hydrogen or Ci-Ce alkyl; X is —CH₂OH, or —CH₂A wherein A is a nitrogen comprising unit, phenyl, or substituted phenyl. Preferred nitrogen comprising A units include amino, pyrrolidino, piperidino, morpholino, piperazino, and mixtures thereof.

Anti-oxidants such as tocopherol sorbate, butylated hydroxyl benxoic acids and their salts, gallic acid and its alkyl esters, ascorbic, citric, tartric, uric acid and its salts, sorbic acid and its salts, and dihydroxyfumaric acid and its salts may also be used. Preferred types of anti-oxidant for use in the composition include 3,5-di-tert-butyl-4-hydroxytoluene (BHT), a-, b-, g-, d-tocopherol, 1,2-benzisothiazoline-3-one (Proxel GXL™), anthocyanins, carotene, catechins, flavonoids, lutein, lycopene and mixtures thereof. Other preferred types of anti-oxidant for use in the composition include hindered phenols, diarylamines (including phenoxazines with a maximum molar extinction coefficient in the wavelength range from 400 to 750 nm of less than 1,000 M⁻¹ ah⁻¹), and mixtures thereof. The number of equivalents of hindered phenol initially formulated can normally be greater than or equal to the number of equivalents of diarylamine

### Source of Hydrogen Peroxide

It may be preferred for the composition to comprise a source of hydrogen peroxide. Sources of hydrogen peroxide include, for example, hydrogen peroxide, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. Percarbonate salts can be preferred. When employed, inorganic perhydrate salts are typically present in amounts of from 0.05 to 40 wt %, or 1 to 30 wt % of the overall dish care product and are typically incorporated into such dish care products as a crystalline solid that may be coated. Suitable coatings include, inorganic salts such as alkali metal silicate, carbonate or borate salts or mixtures thereof, or organic materials such as water-soluble or dispersible polymers, waxes, oils or fatty soaps. These may be present in combination with bleach activators and/or bleach catalysts.

Suitable bleach activators are those having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). While any suitable bleach activator may be employed, it may be preferred if the subject composition comprises NOBS, TAED or mixtures thereof.

Suitable bleach catalysts include one or more bleach catalysts capable of accepting an oxygen atom from a peroxyacid and/or salt thereof and transferring the oxygen atom to an oxidizable substrate. Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and alpha amino-ketones and mixtures thereof.

Suitable bleach catalysts include oxaziridinium bleach catalysts, transition metal bleach catalysts, especially manganese and iron bleach catalysts. A suitable bleach catalyst has a structure corresponding to general formula below:

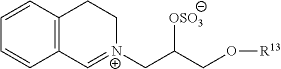

wherein R¹³ is selected from the group consisting of 2-ethylhexyl, 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl.

Another suitable source of hydrogen peroxide includes pre-formed peracids. Suitable preformed peracids include, but are not limited to compounds selected from the group consisting of pre-formed peroxyacids or salts thereof typically a percarboxylic acids and salts, percarbonic acids and salts, perimidic acids and salts, peroxymonosulfuric acids and salts, for example, Oxone®, and mixtures thereof. Suitable examples include peroxycarboxylic acids or salts thereof, or peroxysulphonic acids or salts thereof. Typical peroxycarboxylic acid salts suitable for use herein have a chemical structure corresponding to the following chemical formula:

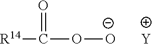

wherein: R¹⁴ is selected from alkyl, aralkyl, cycloalkyl, aryl or heterocyclic groups; the R¹⁴ group can be linear or branched, substituted or unsubstituted; having, when the peracid is hydrophobic, from 6 to 14 carbon atoms, or from 8 to 12 carbon atoms and, when the peracid is hydrophilic, less than 6 carbon atoms or even less than 4 carbon atoms and Y is any suitable counter-ion that achieves electric charge neutrality, preferably Y is selected from hydrogen, sodium or potassium. R¹⁴ may be a linear or branched, substituted or unsubstituted C₆₋₉ alkyl. The peroxyacid or salt thereof may be selected from peroxyhexanoic acid, peroxyheptanoic acid, peroxyoctanoic acid, peroxynonanoic acid, peroxydecanoic acid, any salt thereof, or any combination thereof. Peroxyacids that may be used include phthalimido-peroxy-alkanoic acids, in particular ε-phthalimido peroxy hexanoic acid (PAP). The peroxyacid or salt thereof may have a melting point in the range of from 30° C. to 60° C.

The pre-formed peroxyacid or salt thereof can also be a peroxysulphonic acid or salt thereof, typically having a chemical structure corresponding to the following chemical formula:

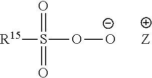

wherein: R¹⁵ is selected from alkyl, aralkyl, cycloalkyl, aryl or heterocyclic groups; the R¹⁵ group can be linear or branched, substituted or unsubstituted; and Z is any suitable counter-ion that achieves electric charge neutrality, preferably Z is selected from hydrogen, sodium or potassium. Preferably R¹⁵ is a linear or branched, substituted or unsubstituted C₄₋₁₄, preferably C₆₋₁₄ alkyl. Preferably such bleach components may be present in the compositions of the invention in an amount from 0.01 to 50%, most preferably from 0.1% to 20% by weight of the detergent composition.

Hydrogen peroxide may also be provided by the incorporation of one or more hydrogen peroxide producing enzymes such as alcohol oxidoreductases, aldehyde oxidoreductases, amino acid oxidoreductases, and monoamine oxidases. These enzymes can convert in situ (e.g. in the washing process) substrates such as carbohydrates, proteins, amino acids, alcohols, amines, or other substrates either from a soil or from a material also present in the composition, to generate hydrogen peroxide. Since this will tend to generate low levels of hydrogen peroxide this may be preferred. Non-limiting examples of hydrogen peroxide producing enzymes are: glycolate oxidases (EC 1.1.3.1), L-lactate oxidases (EC 1.1.3.2), malate oxidases (EC 1.1.3.3), glucose oxidases (EC 1.1.3.4), glycerol oxidases (EC 1.1.3.B4), hexose oxidases (EC 1.1.3.5), cholesterol oxidases (EC 1.1.3.6), aryl-alcohol oxidases (EC 1.1.3.7), L-gulonolactone oxidases (EC 1.1.3.8), galactose oxidases (EC 1.1.3.9), pyranose oxidases (EC 1.1.3.10), L-sorbose oxidases (EC 1.1.3.11), alcohol oxidases (EC 1.1.3.13), (S)-2-hydroxy-acid oxidases (EC 1.1.3.15), chlorine oxidases (EC 1.1.3.17), secondary-alcohol oxidases (EC 1.1.3.18), long-chain-alcohol oxidases (EC 1.1.3.20), thiamine oxidases (EC 1.1.3.23), nucleoside oxidases (EC 1.1.3.28, EC 1.1.3.39), polyvinyl-alcohol oxidases (EC 1.1.3.30), vanillyl-alcohol oxidases (EC 1.1.3.38), D-mannitol oxidase ((EC 1.1.3.40), alditol oxidases (EC 1.1.3.41), glucooligosaccharide oxidases (EC 1.1.99.B3), cellobiose dehydrogenase (EC 1.1.99.18), aldehyde oxidases (EC 1.2.3.1), pyruvate oxidases (EC 1.2.3.3), oxalate oxidases (EC 1.2.3.4), glyoxylate oxidases (EC 1.2.3.5), D-aspartate oxidases (EC 1.4.3.1), L-amino acid oxidases (EC 1.4.3.2), D-amino acid oxidases (EC 1.4.3.3), monoamine oxidases (EC 1.4.3.4), D-glutamate oxidases (EC 1.4.3.7), ethanolamine oxidases (EC 1.4.3.8), protein-lysine 6-oxidases (EC 1.4.3.13), L-lysine oxidases (EC 1.4.3.14), D-glutamate (D-aspartate) oxidases (EC 1.4.3.15), L-aspartate oxidases (EC 1.4.3.16), glycine oxidases (EC 1.4.3.19), L-lysine 6-oxidases (EC 1.4.3.20), primary-amine oxidases (EC 1.4.3.21), diamine oxidases (EC 1.4.3.22), L-arginine oxidases (EC 1.4.3.25), non-specific polyamine oxidases (EC 1.5.3.17), other alcohol oxidoreductases (EC 1.1.X.X), other aldehyde oxidoreductases (EC 1.2.X.X), other amino acid oxidoreductases or monoamine oxidases (EC 1.3.X.X), and other amine oxidoreductases (EC 1.5.X.X). The hydrogen peroxide producing enzyme can be fused to the P450 fatty acid decarboxylase to form a single polypeptide or can be independent enzymes.

The hydrogen peroxide source can be substituted by: a) a source of nicotinamide adenine dinucleotide (NADH) or nicotinamide adenine dinucleotide phosphate (NADPH) and b) an enzymatic redox system. Non-limiting examples of enzymatic redox systems are: the reductase domain of Bacillus megaterium CYP102A1 (P450BM3), the RhFred reductase domain from Rhodococcus sp. NCIMB 9784, the flavodoxin (Fld)/ferrodoxin reductase (FdR, EC 1.18.1.2 and EC 1.18.1.3) system, the putidaredoxin (Pd)/putidaredoxin reductase (PdR, EC 1.18.1.5) system, the rubredoxin/rubredoxin reductase (EC 1.18.1.1 and EC 1.18.1.4) system, and the adrenoxin/adrenodoxin reductase (EC 1.18.1.6) system. In some embodiments, the composition may also comprise a dehydrogenase-based NADH or NADPH regeneration system, such as the phosphonate/phosphonate dehydrogenase (EC 1.20.1.1) system.

### Organic Solvent

The composition of the present invention may optionally comprise an organic solvent. Suitable organic solvents include C4-14 ethers and diethers, polyols, glycols, alkoxylated glycols, C6-C16 glycol ethers, alkoxylated aromatic alcohols, aromatic alcohols, aliphatic linear or branched alcohols, alkoxylated aliphatic linear or branched alcohols, alkoxylated C1-C5 alcohols, C8-C14 alkyl and cycloalkyl hydrocarbons and halohydrocarbons, and mixtures thereof. Preferably the organic solvents include alcohols, glycols, and glycol ethers, alternatively alcohols and glycols. The composition comprises from 0% to less than 50%, preferably from 0.01% to 25%, more preferably from 0.1% to 10%, or most preferably from 0.5% to 5%, by weight of the total composition of an organic solvent, preferably an alcohol, more preferably an ethanol, a polyalkyleneglycol, more preferably polypropyleneglycol, and mixtures thereof.

### Polymer:

The composition can comprise a polymer, preferably at a level of from 0.1% to 5%, more preferably from 0.2% to 3%, even more preferably from 0.3% to 2% by weight of the liquid composition. Suitable polymers can be selected from triblock copolymers, amphiphilic alkoxylated polyalkyleneimine, ethoxylated polyalkyleneimine, polyester soil release polymers, and mixtures thereof, preferably triblock copolymers, amphiphilic alkoxylated polyalkyleneimine, and mixtures thereof.

Suitable triblock copolymers comprise alkylene oxide moieties according to Formula (I): (EO)x(PO)y(EO)x, wherein EO represents ethylene oxide, and each x represents the number of EO units within the EO block. Each x is independently a number average between 3 and 50, preferably between 5 and 25, more preferably between 10 and 15. Preferably x is the same for both EO blocks, wherein the “same” means that the x between the two EO blocks varies within a maximum 2 units, preferably within a maximum of 1 unit, more preferably both x's are the same number of units. PO represents propylene oxide, and y represents the number of PO units in the PO block. Each y is a number average between 5 and 60, preferably between 10 and 40, more preferably between 25 and 35.

The triblock co-polymer can have a ratio of y to each x of from 0.8:1 to 5:1, preferably from 1:1 to 3:1, more preferably from 1.5:1 to 2.5:1. The triblock co-polymer can have an average weight percentage of total EO of between 30% and 50% by weight of the triblock co-polymer. As such, the triblock co-polymer can have an average weight percentage of total PO of between 50% and 70% by weight of the triblock copolymer. It is understood that the average total weight % of EO and PO for the triblock co-polymer adds up to 100%, excluding the end-caps. The end-caps are preferably hydrogen, hydroxyl, methyl, and mixtures thereof, more preferably hydrogen, methyl, and mixtures thereof, and most preferably hydrogen. The triblock co-polymer has a number average molecular weight of between 550 and 8000, preferably between 1000 and 4500, more preferably between 2000 and 3100. Number average molecular weight and compositional analysis of the co-polymer is determined using a 1H NMR spectroscopy (see Thermo scientific application note No. AN52907). It is an established tool for polymer characterization, including number-average molecular weight determination and co-polymer composition analysis.

EO-PO-EO triblock co-polymers are commercially available from BASF such as the Pluronic® PE series, and from the Dow Chemical Company such as Tergitol™ L series. Particularly preferred triblock co-polymer from BASF are sold under the tradenames Pluronic® L44 (MW ca 2200, ca 40 wt % EO), Pluronic® PE6400 (MW ca 2900, ca 40 wt % EO), Pluronic® PE4300 (MW ca 1600, ca 30 wt % EO), and Pluronic® PE 9400 (MW ca 4600, 40 wt % EO). Particularly preferred triblock co-polymer from the Dow Chemical Company is sold under the tradename of Tergitol™ L64 (MW ca 2900, ca 40 wt % EO). The preparation method for such triblock co-polymers is well known to polymer manufacturers.

Suitable amphiphilic polymers can be selected from the group consisting of: amphiphilic alkoxylated polyalkyleneimine and mixtures thereof. Preferably, the amphiphilic alkoxylated polyalkyleneimine is an alkoxylated polyethyleneimine polymer comprising a polyethyleneimine backbone having a weight average molecular weight range of from 100 to 5,000, preferably from 400 to 2,000, more preferably from 400 to 1,000 Daltons. The polyethyleneimine backbone comprises the following modifications:
(i) one or two alkoxylation modifications per nitrogen atom, dependent on whether the modification occurs at an internal nitrogen atom or at an terminal nitrogen atom, in the polyethyleneimine backbone, the alkoxylation modification consisting of the replacement of a hydrogen atom on by a polyalkoxylene chain having an average of about 1 to about 50 alkoxy moieties per modification, wherein the terminal alkoxy moiety of the alkoxylation modification is capped with hydrogen, a C1-C4 alkyl or mixtures thereof;
(ii) a substitution of one C1-C4 alkyl moiety and one or two alkoxylation modifications per nitrogen atom, dependent on whether the substitution occurs at a internal nitrogen atom or at an terminal nitrogen atom, in the polyethyleneimine backbone, the alkoxylation modification consisting of the replacement of a hydrogen atom by a polyalkoxylene chain having an average of about 1 to about 50 alkoxy moieties per modification wherein the terminal alkoxy moiety is capped with hydrogen, a C1-C4 alkyl or mixtures thereof; or
(iii) a combination thereof.

A preferred amphiphilic alkoxylated polyethyleneimine polymer has the general structure of formula (II):

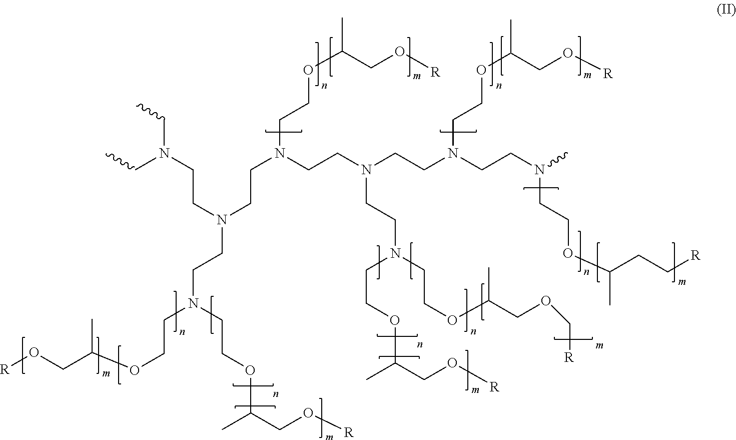

wherein the polyethyleneimine backbone has a weight average molecular weight of about 600, n of formula (II) has an average of about 10, m of formula (II) has an average of about 7 and R of formula (II) is selected from hydrogen, a C1-C4 alkyl and mixtures thereof, preferably hydrogen. The degree of permanent quaternization of formula (II) may be from 0% to about 22% of the polyethyleneimine backbone nitrogen atoms. The molecular weight of this amphiphilic alkoxylated polyethyleneimine polymer preferably is between 10,000 and 15,000 Da.

More preferably, the amphiphilic alkoxylated polyethyleneimine polymer has the general structure of formula (II) but wherein the polyethyleneimine backbone has a weight average molecular weight of about 600 Da, n of Formula (II) has an average of about 24, m of Formula (II) has an average of about 16 and R of Formula (II) is selected from hydrogen, a C1-C4 alkyl and mixtures thereof, preferably hydrogen. The degree of permanent quaternization of Formula (II) may be from 0% to about 22% of the polyethyleneimine backbone nitrogen atoms, and is preferably 0%. The molecular weight of this amphiphilic alkoxylated polyethyleneimine polymer preferably is between 25,000 and 30,000, most preferably 28,000 Da.

The amphiphilic alkoxylated polyethyleneimine polymers can be made by the methods described in more detail in PCT Publication No. WO 2007/135645.

Alternatively, the alkoxylated polyalkyleneimine polymer can be an ethoxylated polyalkyleneimine which comprises no further alkoxylation, and as such, is hydrophilic rather than amphiphilic. That is, the ethoxylated polyalkyleneimine comprises no further alkoxylation such as propoxylation or butoxylation. Preferred ethoxylated polyalkyleneimines consist of alkyleneimine monomer units and ethoxylation (-EO-) monomer units, with the exception of any end-caps, which are typically hydrogen. Ethyleneimine monomer units are highly preferred alkyleneimine monomer units. More preferably, the hydrophilic ethoxylated polyethyleneimine polymer has the general structure of formula (II) but wherein the polyethyleneimine backbone has a weight average molecular weight of about 600 Da, n of Formula (II) has an average of about 20, m of Formula (II) is zero and R of Formula (II) is selected from hydrogen, a C₁-C₄ alkyl and mixtures thereof, preferably hydrogen. The degree of permanent quaternization of Formula (II) may be from 0% to about 22% of the polyethyleneimine backbone nitrogen atoms, and is preferably 0%. The molecular weight of this ethoxylated polyethyleneimine polymer preferably is between 10,000 and 15,000, most preferably 12,600 Da.

Polyester soil release agents are also suitable polymers. Soil release agents are polymers having soil release properties, i.e. having the property to enhance the cleaning efficacy of the detergent composition by improving release of greasy and oil during the laundry process. See soil release agents' definition, p. 278-279, “Liquid Detergents” by Kuo-Yann Lai.

Suitable polyester soil release agents can encompass simple copolymeric blocks of ethylene terephthalate or propylene terephthalate with polyethylene oxide or polypropylene oxide terephthalate (see U.S. Pat. Nos. 3,959,230 and 3,893,929). Other suitable polyester soil release agents can be polyesters with repeat units containing 10-15% by weight of ethylene terephthalate together with 90-80% by weight of polyoxyethylene terephthalate, derived from a polyoxyethylene glycol of average molecular weight 300-5,000. Commercial examples include ZELCON® 5126 from Dupont and MILEASE®T from ICI. Suitable polymeric soil release agents can be prepared by art-recognized methods. U.S. Pat. Nos. 4,702,857 and 4,711,730 describe the preferred method of synthesis for the block polyesters of use.

### Cyclic Polyamine

The composition can comprise a cyclic polyamine having amine functionalities that helps cleaning. The composition of the invention preferably comprises from about 0.1% to about 3%, more preferably from about 0.2% to about 2%, and especially from about 0.5% to about 1%, by weight of the composition, of the cyclic polyamine.

The amine can be subjected to protonation depending on the pH of the cleaning medium in which it is used. Preferred cyclic polyamines have the following Formula (IV):

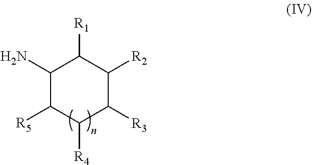

wherein R₁, R₂, R₃, R₄ and R₅ are independently selected from the group consisting of NH2, —H, linear or branched alkyl having from about 1 to about 10 carbon atoms, and linear or branched alkenyl having from about 1 to about 10 carbon atoms, n is from about 1 to about 3, preferably n is 1, and wherein at least one of the Rs is NH2 and the remaining “Rs” are independently selected from the group consisting of NH2, —H, linear or branched alkyl having about 1 to about 10 carbon atoms, and linear or branched alkenyl having from about 1 to about 10 carbon atoms. Preferably, the cyclic polyamine is a diamine, wherein n is 1, R₂ is NH2, and at least one of R₁, R₃, R₄ and R₅ is CH3 and the remaining Rs are H.

The cyclic polyamine has at least two primary amine functionalities. The primary amines can be in any position in the cyclic amine but it has been found that in terms of grease cleaning, better performance is obtained when the primary amines are in positions 1,3. It has also been found that cyclic amines in which one of the substituents is —CH3 and the rest are H provided for improved grease cleaning performance.

Accordingly, the most preferred cyclic polyamine for use with the detergent composition of the present invention are cyclic polyamine selected from the group consisting of: 2-methylcyclohexane-1,3-diamine, 4-methylcyclohexane-1,3-diamine and mixtures thereof. These specific cyclic polyamines work to improve suds and grease cleaning profile through-out the dishwashing process when formulated together with the surfactant system of the composition of the present invention.

### Chelant

The detergent composition herein can comprise a chelant at a level of from 0.1% to 20%, preferably from 0.2% to 5%, more preferably from 0.2% to 3% by weight of total composition.

As commonly understood in the detergent field, chelation herein means the binding or complexation of a bi- or multidentate ligand. These ligands, which are often organic compounds, are called chelants, chelators, chelating agents, and/or sequestering agent. Chelating agents form multiple bonds with a single metal ion. Chelants, are chemicals that form soluble, complex molecules with certain metal ions, inactivating the ions so that they cannot normally react with other elements or ions to produce precipitates or scale, or forming encrustations on soils turning them harder to be removed. The ligand forms a chelate complex with the substrate. The term is reserved for complexes in which the metal ion is bound to two or more atoms of the chelant.

Preferably, the composition of the present invention comprises one or more chelant, preferably selected from the group comprising carboxylate chelants, amino carboxylate chelants, amino phosphonate chelants such as MGDA (methylglycine-N,N-diacetic acid), GLDA (glutamic-N,N-diacetic acid), and mixtures thereof.

Suitable chelating agents can be selected from the group consisting of amino carboxylates, amino phosphonates, polycarboxylate chelating agents and mixtures thereof.

Other chelants include homopolymers and copolymers of polycarboxylic acids and their partially or completely neutralized salts, monomeric polycarboxylic acids and hydroxycarboxylic acids and their salts. Suitable polycarboxylic acids are acyclic, alicyclic, heterocyclic and aromatic carboxylic acids, in which case they contain at least two carboxyl groups which are in each case separated from one another by, preferably, no more than two carbon atoms. A suitable hydroxycarboxylic acid is, for example, citric acid. Another suitable polycarboxylic acid is the homopolymer of acrylic acid. Preferred are the polycarboxylates end capped with sulfonates.

### Method of Washing

Other aspects of the invention are directed to methods of washing ware especially dishware with a composition of the present invention. Accordingly, there is provided a method of manually washing dishware comprising the steps of delivering a hand-dishwashing composition of the invention into a volume of water to form a wash solution and immersing the dishware in the solution. Preferably the P450 fatty acid decarboxylase is present at a concentration from 0.005 ppm to 15 ppm, preferably from 0.02 ppm to 0.5 ppm, in an aqueous wash liquor during the washing process. As such, the composition herein will be applied in its diluted form to the dishware. Soiled surfaces e.g. dishes are contacted with an effective amount, typically from 0.5 mL to 20 mL (per 25 dishes being treated), preferably from 3 mL to 10 mL, of the detergent composition of the present invention, preferably in liquid form, diluted in water. The actual amount of detergent composition used will be based on the judgment of user, and will typically depend upon factors such as the particular product formulation of the composition, including the concentration of active ingredients in the composition, the number of soiled dishes to be cleaned, the degree of soiling on the dishes, and the like. Generally, from 0.01 mL to 150 mL, preferably from 3 mL to 40 mL of a liquid detergent composition of the invention is combined with from 2,000 mL to 20,000 mL, more typically from 5,000 mL to 15,000 mL of water in a sink having a volumetric capacity in the range of from 1,000 mL to 20,000 mL, more typically from 5,000 mL to 15,000 mL. The soiled dishes are immersed in the sink containing the diluted compositions then obtained, where contacting the soiled surface of the dish with a cloth, sponge, or similar article cleans them. The cloth, sponge, or similar article may be immersed in the detergent composition and water mixture prior to being contacted with the dish surface, and is typically contacted with the dish surface for a period of time ranged from 1 to 10 seconds, although the actual time will vary with each application and user. The contacting of cloth, sponge, or similar article to the surface is preferably accompanied by a concurrent scrubbing of the surface.

Alternatively, the dishwashing composition can be applied directly onto a cleaning implement or the dishes to be cleaned without any pre-dilution step, or with slight dissolutions as is the case when applied using a damp sponge or other implement.

### Test Methods

The following assays set forth must be used in order that the invention described and claimed herein may be more fully understood.

### Test Method 1—Enzyme Activity Assay for P450 Fatty Acid Decarboxylases

Enzymatic reactions with P450 fatty acid decarboxylases can be performed as follows. Aliquots of sodium salts of fatty acids (e.g. sodium palmitate, sodium stearate, sodium oleate, sodium linoleate, or sodium linolenate; final concentration 100-200 μM) and FAD (final concentration 200 μM) are resuspended in a suitable reaction buffer (pH 7 to 9). The reaction is started by addition of the enzyme (final concentration 1 μM) and the solutions are incubated for up to 240 minutes at a suitable temperature. Aliquots of 100 μL of the reaction solutions are collected at different time points and mixed with 900 μL of isopropyl alcohol to stop the reaction. Analysis of the samples is performed by reversed-phase LC/MS/MS or GC/MS using standard procedures known in the art to determine the concentrations of salts of fatty acid remaining in the solutions and the percent conversion is calculated. As used herein, a P450 fatty acid decarboxylase catalyzes the conversion of a fatty acid when the percent conversion of said fatty acid is at least 5% under optimal reaction conditions in 240 minutes or less time.

### EXAMPLES

Hereinafter, the present invention is described in more detail based on examples. All percentages are by weight unless otherwise specified.

### Comparative Example A—Production of Jeotgalicoccus Sp. OleTJE

Jeotgalicoccus sp. OleTJE (SEQ ID NO: 1) is a P450 fatty acid decarboxylase that converts medium chain fatty acids (e.g. linoleic acid) into the corresponding terminal olefins and is included as a comparative example of the present invention. A codon optimized gene (SEQ ID NO: 152) encoding for a Jeotgalicoccus sp. OleTJE variant, including an N-terminal amino acid sequence containing a His-tag and a TEV protease cleavage site (SEQ ID NO: 153), was designed and synthesized. After gene synthesis, the protein was expressed and purified. In brief, the complete synthetic gene sequence was subcloned into a pET30a vector by GenScript (Piscataway, N.J.). For heterologous expression. Escherichia coli BL21 Star™ (DE3) pLysS cells were transformed with the recombinant plasmid and a single colony was inoculated into LB medium containing kanamycin and chloramphenicol. Pre-starter cultures were then inoculated into a fermentor (BioFlo/CelliGen 310; NewBrunswick, Hamburg, Germany) containing LB medium supplemented with kanamycin and chloramphenicol and incubated at 25° C. At an OD_{600 nm}=0.4, isopropyl β-D-1-thiogalactopyranoside (IPTG) (final concentration 0.1 mM) and 5-aminolevulinic acid (final concentration 0.5 mM) were added to induce protein expression. Cells were harvested by centrifugation at 5000 rpm and 4° C. and the pellets were lysed by a bacterial cell lysis buffer (B-PER—ThemoFisher, Waltham, Mass.). After centrifugation, the supernatant was collected, and the protein was purified by one-step purification using Ni-NTA agarose resin (Qiagen, Hilden, Germany; catalog #30230) and standard protocols known in the art. The protein was dialyzed using a membrane with 10 kDa MW cutoff against a buffer containing 50 mM Tris-HCl and 10% Glycerol at pH 8.0. The final protein concentration was 1.1 mg/mL as determined by Modified Lowry protein assay with BSA as a standard (ThermoFisher Scientific, Waltham, Mass.).

### Example 1—Production of Micrococcus lylae OleTML

Micrococcus lylae OleTML (SEQ ID NO: 2) is a P450 fatty acid decarboxylase that converts medium chain fatty acids (e.g. linoleic acid) into the corresponding terminal olefins and that is included as an example of the current invention. A codon optimized gene (SEQ ID NO: 154) encoding for a Micrococcus lylae OleTML variant (SEQ ID NO: 155), including an N-terminal amino acid sequence containing a His-tag and a TEV protease cleavage site, was designed and synthesized. After gene synthesis, the protein was expressed and purified by Genscript. In brief, the complete synthetic gene sequence was subcloned into a pET30a vector for heterologous expression. Escherichia coli C41 (DE3) cells were co-transformed with the recombinant plasmid and with plasmid pTf16. A single colony was inoculated into TB medium containing kanamycin and chloramphenicol. Cultures were incubated at 15° C. for 16 h at 200 rpm and L-arabinose (final concentration 0.1%), δ-aminolevulinic acid (final concentration 0.25 mM) and isopropyl β-D-1-thiogalactopyranoside (IPTG, final concentration 1 mM) were added to induce protein expression. Cells were harvested by centrifugation at 5000 rpm and 4° C. and the pellets were lysed by sonication. After centrifugation, the supernatant was collected and the protein was purified by one-step purification using a nickel affinity column and standard protocols known in the art. The protein was stored in a buffer containing 50 mM Tris-HCl, 500 mM NaCl, and 10% Glycerol at pH 8.0. The final protein concentration was 0.93 mg/mL as determined by Bradford protein assay with BSA as a standard (ThermoFisher, catalog #23236).

### Example 2—Production of Macrococcus bovicus OleTMB

Macrococcus bovicus OleTMB (SEQ ID NO: 22) is a predicted P450 fatty acid decarboxylase that converts medium chain fatty acids (e.g. linoleic acid) into the corresponding terminal olefins and that is included as an example of the current invention. A codon optimized gene (SEQ ID NO: 159) encoding for an OleTMB decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigm, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 3—Production of Staphylococcus delphini OleTSD

Staphylococcus delphini OleTSD (SEQ ID NO: 44) is a predicted P450 fatty acid decarboxylase that converts medium chain fatty acids (e.g. linoleic acid) into the corresponding terminal olefins and that is included as an example of the current invention. A codon optimized gene (SEQ ID NO: 160) encoding for an OleTSD decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigma, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 4—Production of Staphylococcus felis OleTSF

Staphylococcus felis OleTSF (SEQ ID NO: 60) is a predicted P450 fatty acid decarboxylase that converts medium chain fatty acids (e.g. palmitic acid) into the corresponding terminal olefins and that is included as an example of the current invention. A codon optimized gene (SEQ ID NO: 161) encoding for an OleTSF decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigma, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 5—Production of Fictibacillus Sp. S7 OleTFS

Fictibacillus sp. S7 OleTFS (SEQ ID NO: 65) is a predicted P450 fatty acid decarboxylase that converts medium chain fatty acids (e.g. palmitic acid) into the corresponding terminal olefins and that is included as an example of the current invention.v A codon optimized gene (SEQ ID NO: 162) encoding for an OleTFS decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigma, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 6—Production of Staphylococcus aureus C0673 OleTSA

Staphylococcus aureus C0673 OleTSA (SEQ ID NO: 71) is a predicted P450 fatty acid decarboxylase that converts medium chain fatty acids (e.g. palmitic acid) into the corresponding terminal olefins and that is included as an example of the current invention. A codon optimized gene (SEQ ID NO: 163) encoding for an OleTSA decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigma, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 7—Production of Auricoccus indicus OleTAI

Auricoccus indicus OleTAI (SEQ ID NO: 83) is a predicted P450 fatty acid decarboxylase that converts medium chain fatty acids (e.g. palmitic acid) into the corresponding terminal olefins and that is included as an example of the current invention. A codon optimized gene (SEQ ID NO: 164) encoding for an OleTAI decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigma, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 8—Production of Nosocomiicoccus massiliensis OleTNM

Nosocomiicoccus massiliensis OleTNM (SEQ ID NO: 117) is a predicted P450 fatty acid decarboxylase that converts medium chain fatty acids (e.g. palmitic acid) into the corresponding terminal olefins and that is included as an example of the current invention. A codon optimized gene (SEQ ID NO: 165) encoding for an OleTNM decarboxylase variant), including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigma, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 9—Production of Pontibacillus halophilus JSM 076056 OleTPH

Pontibacillus halophilus JSM 076056 OleTPH (SEQ ID NO: 121) is a predicted P450 fatty acid decarboxylase that converts medium chain fatty acids (e.g. palmitic acid) into the corresponding terminal olefins and that is included as an example of the current invention. A codon optimized gene (SEQ ID NO: 166) encoding for an OleTPH decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigma, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 10—Production of Macrococcus sp. DPC7161 OleTMS

Macrococcus sp. DPC7161 OleTMS (SEQ ID NO: 122) is a predicted P450 fatty acid decarboxylase that converts medium chain fatty acids (e.g. palmitic acid) into the corresponding terminal olefins and that is included as an example of the current invention. A codon optimized gene (SEQ ID NO: 167) encoding for an OleTMS decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigma, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 11—Production of Staphylococcus massiliensis S46 OleTSM

Staphylococcus massiliensis S46 OleTSM (SEQ ID NO: 156) is a P450 fatty acid decarboxylase that converts medium chain fatty acids (e.g. palmitic acid) into the corresponding terminal olefins and that is included as an example of the current invention. A codon optimized gene (SEQ ID NO: 168) encoding for an OleTSM decarboxylase variant, without the initial N-terminal 29 amino acids including an N-terminal amino acid sequence containing a His-tag and a TEV protease cleavage site was designed and synthesized by Genscript.

In brief, the complete synthetic gene sequence was subcloned into a pET30a vector using the NdeI and HindIII cloning sites for heterologous expression. Escherichia coli C41 (DE3) cells were co-transformed with the recombinant plasmid and with plasmid pTf16. A single colony was inoculated into TB medium containing kanamycin and chloramphenicol. Cultures were incubated at 15° C. for 16 h at 200 rpm and L-arabinose (final concentration 0.1%), δ-aminolevulinic acid (final concentration 0.25 mM) and isopropyl β-D-1-thiogalactopyranoside (IPTG, final concentration 1 mM) were added to induce protein expression. Cells were harvested by centrifugation at 5000 rpm and 4° C. and the pellets were lysed by sonication. After centrifugation, the supernatant was collected and the protein was purified by one-step purification using a nickel affinity column and standard protocols known in the art. The protein was stored in a buffer containing 50 mM Tris-HCl, 500 mM NaCl, and 10% Glycerol at pH 8.0. The final protein concentration was 1.65 mg/mL as determined by Bradford protein assay with BSA as a standard (ThermoFisher, catalog #23236).

### Comparative Example B—Production of Macrococcus goetzii OleTMG

Macrococcus goetzii OleTMG (SEQ ID NO: 20) is a predicted P450 fatty acid decarboxylase that converts fatty acids into the corresponding terminal olefins and is included as a comparative example of the present invention. A codon optimized gene (SEQ ID NO: 169) encoding for an OleTMG decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigm, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Comparative Example C—Production of Macrococcus lamae OleTMA

Macrococcus lamae OleTMA (SEQ ID NO: 21) is a predicted P450 fatty acid decarboxylase that converts fatty acids into the corresponding terminal olefins and is included as a comparative example of the present invention. A codon optimized gene (SEQ ID NO: 170) encoding for an OleTMA decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigm, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Comparative Example D—Production of Salinicoccus sp. CT19 OleTSS

Salinicoccus sp. CT19 OleTSS (SEQ ID NO: 42) is a predicted P450 fatty acid decarboxylase that converts fatty acids into the corresponding terminal olefins and is included as a comparative example of the present invention. A codon optimized gene (SEQ ID NO: 171) encoding for an OleTSS decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigm, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Comparative Example E—Production of Aliicoccus persicus OleTAP

Aliicoccus persicus OleTAP (SEQ ID NO: 84) is a predicted P450 fatty acid decarboxylase that converts fatty acids into the corresponding terminal olefins and is included as a comparative example of the present invention. A codon optimized gene (SEQ ID NO: 172) encoding for an OleTAP decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigm, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Comparative Example F—Production of Salinicoccus qingdaonensis OleTSQ

Salinicoccus qingdaonensis OleTSQ (SEQ ID NO: 100) is a predicted P450 fatty acid decarboxylase that converts fatty acids into the corresponding terminal olefins and is included as a comparative example of the present invention. A codon optimized gene (SEQ ID NO: 173) encoding for an OleTSQ decarboxylase variant, including an N-terminal amino acid sequence containing a His-tag and a TEV protease cleavage site was designed and synthesized by Genscript. After gene synthesis, the protein was expressed and purified. In brief, the complete synthetic gene sequence was subcloned into a pET30a using the NdeI/XhoI cloning sites. For heterologous expression, Escherichia coli BL21 (DE3) cells were transformed with the recombinant plasmid and a single colony was inoculated into LB medium containing kanamycin (50 mg/L). Pre-starter cultures were then inoculated into a fermentor containing Magic Media (ThermoFisher, Catalog #K6803) supplemented with kanamycin (50 mg/L) and incubated at 16° C. for 72 h. At an OD_{600 nm}=0.5-1.0, 5-aminolevulinic acid (final concentration 0.5 mM) was added. Cells were harvested by centrifugation at 5000 rpm and 4° C. and the pellets were lysed using a bacterial cell lysis buffer (B-PER—ThemoFisher, Waltham, Mass.). After centrifugation, the supernatant was collected, and the protein was purified by one-step purification using a HisPur™ Ni-NTA Spin Columns (Thermo Scientific, Catalog #88226) and standard protocols known in the art. The protein was concentrated using a 10 kDa MW cutoff Amicon Ultra Centrifugal Filter Unit (MilliporeSigm, Catalog #UFC901024), followed by desalting using a disposable PD-10 desalting column (GE Healthcare Life Sciences, Catalog #17085101) and a buffer containing 50 mM Tris-HCl, 500 mM NaCl, and 10% Glycerol at pH 8.0. The purified enzyme was stored at −80° C. until use.

### Example 12—Enzyme Activity Assay

Reactions of fatty acids with the earlier described OleT enzymes produced as described in examples 1 to 11 and comparative example A, were performed as follows. Aliquots of sodium oleate or sodium linoleate (final concentration 100 μM) and enzyme (final concentration 6 ppm) were resuspended in buffer (50 mM phosphate, 500 mM NaCl, and 10% glycerol at pH 7.4). The reaction was started by addition of hydrogen peroxide (final concentration 220 μM) and the solutions were incubated at 30° C. Aliquots of 100 μL of the reaction solutions were collected at different time points and mixed with 900 μL of isopropyl alcohol to stop the reactions. Analysis of the samples was performed by reversed-phase LC/MS/MS to determine the concentrations of oleate remaining in the solutions. The TON numbers (in s⁻¹) were calculated as the ratio between the initial rate of substrate conversion (in μM/s) and the concentration of enzyme (in μM). Finally, the improvement factors were calculated as the ratio of the TON number for the specific enzyme and the TON number for Jeotgalicoccus sp. OleTJE (SEQ ID NO: 1). The results are summarized in Table 2.

TABLE 2
Conversion of sodium oleate by OleT
decarboxylases at different time points.

| SEQ | | Linoleic- | Oleic- |
|---|---|---|---|
| ID | | Improv. | Improv. |
| NO: | Organism | Factor | Factor |
| 1* | Jeotgalicoccus sp. | 1.00 | 1.00 |
| 2 | Micrococcus lylae | 86.50 | 76.67 |
| 22 | Macrococcus bovicus | 0.00 | 2.00 |
| 44 | Staphylococcus delphini | 2.55 | 0.86 |
| 60 | Staphylococcus felis | 24.00 | 0.10 |
| 65 | Fictibacillus sp. S7 | 20.50 | 5.11 |
| 71 | Staphylococcus aureus C0673 | 10.35 | 3.22 |
| 83 | Auricoccus indicus | 12.00 | 0.33 |
| 117 | Nosocomiicoccus massiliensis | 5.00 | 0.77 |
| 121 | Pontibacillus halophilus | 0.00 | 2.33 |
| | JSM076056 | | |
| 122 | Macrococcus sp. DPC7161 | 42.00 | 18.44 |
| 156 | Staphylococcus massiliensis S46 | 3.75 | 2.22 |
| 20* | Macrococcus goetzii | 0.00 | 1.44 |
| 21* | Macrococcus lamae | 0.00 | 1.39 |
| 42* | Salinicoccus sp. CT19 | 0.00 | 1.33 |
| 84* | Aliicoccus persicus | 0.00 | 1.11 |
| 100* | Salinicoccus qingdaonensis | 0.00 | 0.43 |

*Comparative

### Example 13. Exemplary Manual Dish-Washing Detergent Composition

| Ingredient (levels on active basis) | 2A | 2B | 2C |
|---|---|---|---|
| Sodium alkyl ethoxy sulfate | 22.91% | 22.91% | 22.91% |
| (C1213EO0.6S) | | | |
| n-C12-14 Di Methyl Amine Oxide | 7.64% | 7.64% | 7.64% |
| Lutensol XP80 (non-ionic surfactant | 0.45% | 0.45% | 0.45% |
| supplied by BASF) | | | |
| Sodium Chloride | 1.2% | 1.2% | 1.2% |
| Poly Propylene Glycol (weight | 1% | 1% | 1% |
| average molecular wt. 2000) | | | |
| Ethanol | 2% | 2% | 2% |
| Sodium Hydroxide | 0.24% | 0.24% | 0.24% |
| P450 fatty acid decarboxylase | 0.1% | 0.1% | 0.1% |
| (SEQ ID NO: 2) | | | |
| Hydrogen peroxide | 2.3% | 5.7% | 0.0% |
| Sodium percarbonate | 0.0% | 0.0% | 5.0% |
| Minors (perfume, preservative, | To | To | To |
| dye) + water | 100% | 100% | 100% |

All percentages and ratios given for enzymes are based on active protein. All percentages and ratios herein are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hand-dishwashing composition which provides good sudsing and a good suds profile even in the presence of greasy stains comprising higher chain-length saturated and/or unsaturated fatty acid chains, the composition comprising:
a) a surfactant system comprising at least one anionic surfactant; and
b) a P450 fatty acid decarboxylase, wherein said decarboxylase comprises a polypeptide sequence having at least about 80% identity to one or more sequences selected from the group consisting of: SEQ ID NO: 2, 60, 65, 71, 83, 122, and their functional fragments thereof.

2. The composition according to claim 1, wherein said P450 fatty acid decarboxylase comprises a polypeptide sequence having at least about 90% identity to one or more sequences selected from the group consisting of: SEQ ID NO: 2, 60, 65, 71, 83, 122, and their functional fragments thereof.

3. The composition according to claim 2, wherein said P450 fatty acid decarboxylase is selected from the group consisting of: SEQ ID NO: 2, 122, and their functional fragments.

4. The composition according to claim 1, wherein the P450 fatty acid decarboxylase is present in an amount of from about 0.0001% to about 1% by weight of the hand dish-washing composition, based on active protein.

5. The composition according to claim 4, wherein the P450 fatty acid decarboxylase is present in an amount of from about 0.001% to about 0.2% by weight of the hand dish-washing composition, based on active protein.

6. The composition according to claim 1, wherein the P450 fatty acid decarboxylase comprises an amino acid selected from the group consisting of: a) valine at position 40, b) alanine, glycine, valine, or isoleucine at position 46, c) valine at position 74, d) lysine at position 252, e) leucine at position 317, and combinations thereof; wherein said positions are numbered with reference to SEQ ID NO: 1.

7. The composition according to claim 1, wherein the composition further comprises one or more co-enzymes selected from the group consisting of: fatty-acid peroxidases (EC 1.11.1.3), unspecific peroxygenases (EC 1.11.2.1), plant seed peroxygenases (EC 1.11.2.3), fatty acid peroxygenases (EC1.11.2.4), linoleate diol synthases (EC 1.13.11.44), 5,8-linoleate diol synthases (EC 1.13.11.60 and EC 5.4.4.5), 7,8-linoleate diol synthases (EC 1.13.11.60 and EC 5.4.4.6), 9,14-linoleate diol synthases (EC 1.13.11.B1), 8,11-linoleate diol synthases, oleate diol synthases, other linoleate diol synthases, unspecific monooxygenase (EC 1.14.14.1), alkane 1-monooxygenase (EC 1.14.15.3), oleate 12-hydroxylases (EC 1.14.18.4), fatty acid amide hydrolases (EC 3.5.1.99), fatty acid photodecarboxylases (EC 4.1.1.106), oleate hydratases (EC 4.2.1.53), linoleate isomerases (EC 5.2.1.5), linoleate (10E,12Z)-isomerases (EC 5.3.3.B2), non-heme fatty acid decarboxylases (UndA-like), alpha-dioxygenases, amylases, lipases, proteases, cellulases, and mixtures thereof.

8. The composition according to claim 7, wherein the composition further comprises one or more co-enzymes selected from the group consisting of: fatty-acid peroxidases (EC 1.11.1.3), unspecific peroxygenases (EC 1.11.2.1), plant seed peroxygenases (EC 1.11.2.3), and fatty acid peroxygenases (EC1.11.2.4), non-heme fatty acid decarboxylases (UndA-like), alpha-dioxygenases, and mixtures thereof.

9. The composition according to claim 1, wherein the composition comprises from about 5% to about 50% by weight of the total composition of a surfactant system.

10. The composition according to claim 1, wherein the anionic surfactant comprises alkyl sulphated anionic surfactant selected from the group consisting of: alkyl sulphate, alkyl alkoxy sulphate, and mixtures thereof.

11. The composition according to claim 7, wherein the alkyl sulphated anionic surfactant has an average alkyl chain length of from 8 to 18 carbon atoms.

12. The composition according to claim 7, wherein the alkyl sulphated anionic surfactant has an average degree of alkoxylation, of less than about 5.

13. The composition according to claim 7, wherein the alkyl sulphated anionic surfactant has a weight average degree of branching of more than about 10%.

14. The composition according to a claim 1, wherein the surfactant system further comprises a co-surfactant, wherein the co-surfactant is selected from the group consisting of: an amphoteric surfactant, a zwitterionic surfactant, and mixtures thereof.

15. The composition according to claim 14, wherein the co-surfactant is an amphoteric surfactant.

16. The composition according to claim 15, wherein the amphoteric surfactant is an amine oxide surfactant selected from the group consisting of: alkyl dimethyl amine oxide, alkyl amido propyl dimethyl amine oxide, and mixtures thereof

17. The composition according to claim 1, wherein the composition comprises a source of hydrogen peroxide.

18. The composition according to claim 12, wherein the hydrogen peroxide is selected from the group consisting or: hydrogen peroxide, inorganic perhydrate salts, percarbonate, persulphate, perphosphate, persilicate salts, and mixtures thereof.

19. A method of manually washing dishware comprising the steps of delivering a detergent composition according to claim 1 into a volume of water to form a wash solution and immersing the dishware in the solution.

20. The method according to claim 19, wherein the P450 fatty acid decarboxylase is present at a concentration of from about 0.005 ppm to about 15 ppm in an aqueous wash liquor during the washing process.


SEQUENCE LISTING (173 sequences)

SEQ ID NO: 1 - protein, 422 aa, Jeotgalicoccus sp.
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 2 - protein, 425 aa, Micrococcus lylae
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 3 - protein, 425 aa, Unknown
  Codon optimized artificial DNA sequence
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 4 - protein, 425 aa, Macrococcus caseolyticus
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKVLFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 5 - protein, 425 aa, Macrococcus caseolyticus
MSKRVPKDRGIDNSLKIMKEGYEYVPARMKKFNTNIFETRVLGGKTAVVISGKEAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 6 - protein, 425 aa, Macrococcus caseolyticus
MSKRVPKDRGIDNSLKIMKEGYEYVPARMKKFNTNIFETRVLGGKTAVVISGKDAAKLFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEEMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 7 - protein, 425 aa, Macrococcus caseolyticus
MSKRIPKDRGIDNSLKIMKEGYEYVPARMKKFNTNIFETRVLGGKTAVVISGKDAAELFYDNDKTERKGTLPKRVVKTLFGKGAIHTTTGKKHIDRKVLFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTK

SEQ ID NO: 8 - protein, 425 aa, Macrococcus caseolyticus
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFNGYKIQKDTMMLLDIYGTLHRDDLFSEPERFNPYRFENWDGSPFDLIPQGGGDYYTNHRCAGEWMTIIIMEETMKFFANEISYDVPPQDFTVDTTNFPGKVASGMDIENIRVNIDRTK

SEQ ID NO: 9 - protein, 425 aa, Macrococcus caseolyticus
MSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGITDNPDNIKKMADDMNKMIDSFSAIGSLYGGYREAKKARARVEQFLEDQITAVRKGKIHPEKGTALYEFSHWEDMNEKPMDARLCAVDLMNVIRPLVAINKFVSFGVLALHEFPGERVRVALNEEDYAYKFVQEVRRYYPFVPFLPGKAKENITFDGYKIQKDTMMILDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTQ

SEQ ID NO: 10 - protein, 425 aa, Macrococcus caseolyticus
MSKRIPKDRGIDNSFKIMKEGYEYVPARMKKFNTNIFETRVLGGKAAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGITDNPDNIKKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIQKDTMMILDIYGTLHRDDLFSEPERFNPYRFENWDGSPFDLIPQGGGDYYTNHRCAGEWMTIIIMEETMKFFANEISYDVPPQDFTVDTTKFPGKVASGMDIENIRVNIDRTQ

SEQ ID NO: 11 - protein, 425 aa, Macrococcus canis
MSKRIPKDRGIDNSLKIMKEGYEYVPARMKKFNTNIFETHVLGGKTAVVISGKEAAELFYDNDKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDNIKKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTMMILDIYGTLHRDDLFSEPDRFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 12 - protein, 425 aa, Macrococcus canis
MSKRIPKDRGIDNSLKIMKEGYEYVPARMKKFNTNIFETHVLGGKTAVVISGKEAAELFYDNDKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDNIKKMADDMNKMIDSFSAIGSLYGGYREAKKARARVEQFLEDQITAVRKGKLHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTLMILDIYGTLHRDDLFSEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 13 - protein, 425 aa, Macrococcus canis
MSKRIPKDRGIDNSLKIMKEGYEYVPARMKKFNTNIFETHVLGGKTAVVISGKEAAELFYDNDKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDNIKKMADDMNKMIDSFSAIGSLYGGYREAKKARARVEQFLEDQITAVRKGKLHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTLMILDIYGTLHRDDLFGEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 14 - protein, 425 aa, Macrococcus canis
MSKRIPKDRGIDNSLKIMKEGYEYVPARMKKFNTNIFETHVLGGKTAVVISGKEAAELFYDNNKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDNIKKMADDMNKMIDSFSAIGSLYGGYREAKKARARVEQFLEDQITAVRKGKLHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTLMILDIYGTLHRDDLFGEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 15 - protein, 425 aa, Macrococcus canis
MSKRIPKDRGIDNSLKIMKEGYEYVPARMKKFNTNIFETHVLGGKTAVVISGKEAAELFYDNDKTERKGTLPKRVVKTLFGKGAIHTTAGKTHIDRKALFMSLMTDENLAYLRKLTRNYWFQNIEHMQYKQKVNVYEEATELLTKVGLRWAGIVDNPDSIKKMADDMNKMIDSFSAIGSLYGGYREAKKARARVEQFLEDQITAVRKGKLHPEKGTALYEFSHWEDMNGKPMDARLCAVDLMNVIRPLVAINKFVSFGVLALHEFPGERVRVALNEEDYAYKFVQEVRRYYPFVPFLPGKAKENITFDGYKIKKDTLMILDIYGTLHRDDLFGEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 16 - protein, 425 aa, Macrococcus sp.
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNVYKESIILLTKVGFRWAGIIETPENIEQHAMDMDKMIDSFGAIGSPYKGYREAKKARARVEDFLEEQIIKTRKGKIHPEPGSALYEFSHWEDMNGKPMDSRLCSVDLMNVIRPLVAINRFVAYGVLAMHEFPGERTRVARDDNDYAYKFVQEVRRYYPFVPFLPGKAAKNIEFDGYKIKKDTMIILDVFGTLHREDTFSEPERFNPYRFDNWDGSPFDLIPQGGGDYYTNHRCAGEWMTIIIMEETMKYFAQEIDYDVPPQDFTVDRTKLPGRVKSGMVINNVRLNIDRTK

SEQ ID NO: 17 - protein, 425 aa, Macrococcus goetzii
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNVYKESIILLTKVGFRWAGIIETPENIEQHAMDMDKMIDSFGAIGSPYKGYREAKKARARVEDFLEEQIIKTRKGKIHPEPGSALYEFSHWEDMNGKPMDSRLCSVDLMNVIRPLVAINRFVAYGVLAMHEFPGERTRVARDDNDYAYKFVQEVRRYYPFVPFLPGKAAKNIEFDGYKIKKDTMIILDVFGTLHREDTFSEPERFNPYRFDNWDGSPFDLIPQGGGDYHTNHRCAGEWMTIIIMEETMKYFAQEINYDVPPQDFTVDRTKLPGRVKSGMIINNVRLNIDRTK

SEQ ID NO: 18 - protein, 425 aa, Macrococcus goetzii
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNVYKESIILLTKVGFRWAGIIETPENIEQHAMDMDKMIDSFGAIGSPYKGYREAKKARARVEDFLEEQIIKTRKGKIHPEPGSALYEFSHWEDMNGKPMDSRLCSVDLMNVIRPLVAINRFVAYGVLAMHEFPGERTRVARDDDDYAYKFVQEVRRYYPFVPFLPGKAAKNIEFDGYKIKKDTMIILDVFGTLHREDTFSEPERFNPYRFDNWDGSPFDLIPQGGGDYHTNHRCAGEWMTIIIMEETMKYFAQEIDYDVPPQDFTVDRTKLPGRVKSGMIINNVRLNIDRTK

SEQ ID NO: 19 - protein, 425 aa, Macrococcus epidermidis
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNIYKESIILLTKVGFRWAGIIETPENIEQHAMDMDKMIDSFGAIGSPYKGYREAKKARARVEDFLEEQIIKTRKGKIHPEPGSALYEFSHWEDMNGKPMDSRLCSVDLMNVIRPLVAINRFVAYGVLAMHEFPGERTRVARNDNDYAYKFVQEVRRYYPFVPFLPGKAAKNIEFDGYKIKKDTMIILDVFGTLHREDTFSEPERFNPYRFDNWDGSPFDLIPQGGGDYHTNHRCAGEWMTIIIMEETMKYFAQEINYDVPPQDFTVDRTKLPGRVKSGMIINNVRLNIDRTK

SEQ ID NO: 20 - protein, 425 aa, Macrococcus goetzii
MAKRMPKDRGLDNTLTIMKEGYEYIQNRTKKFNSNVFETRVLGGKQAVVISGKQAAELFYDNDKIERSGTLPPRVVKTLFGKGAIHTTTGKKHIDRKALFMSLMTDENLTYLRKLTRNYWFQNTQRMQLMDEVNVYKESIILLTKIGFRWAGIIETPENIEQHAMDMDKMIDSFGAIGSPYKGYREAKKARTRVEDFLEEQIIKTRKGKIHPEPGSALYEFSHWEDMNGQPMDSRLCSVDLMNVIRPLVAINRFVAYGVLAMHEFPGERTRVARDDDDYAYKFVQEVRRYYPFVPFLPGKAAKNIEFDGYKIKKDTMIILDVFGTLHREDTFSEPERFNPYRFDNWDGSPFDLIPQGGGDYHTNHRCAGEWMTIIIMEETMKYFAQEIDYDVPPQDFTVDRTKLPGRVKSGMIINNVRLNIDRTK

SEQ ID NO: 21 - protein, 425 aa, Macrococcus lamae
MSKPIPVDKGIDKTLTVMKEGYEYVSNRTKKFNSDIFETRVLGGKRAMVISGKEAAELFYDNDKIERKGTLPPRVVKTLFGKGAIHTTTGKVHIDRKSLFMSLMTENNLEYLRKLTRNFWYQNTERMQLMGKVNVYKESIILLTKVGFRWAGVTEKPEKIEQHAMDMDKMIDSFGSVGTAYKGYREAKKARARVEDFLEKQIIATRKGKIHPEEGTALHSFSHWRDFQGNYMDSRLAAVDLMNVIRPLVAINRFVAYGVLALHEFPGERERVSADENNYAYKFTQELRRYFPFVPFLPAKAKVDIDFQGHVIPQDTMIVLDIFGTLHREDLWEDPERFYPDRFENWDGSPFDLIPQGGGDYHTNHRCAGEWMTIIIMEETMKYFAREITYDVPAQDFTVDRTKLPGRVKSGMDIENVRLNFDRTL

SEQ ID NO: 22 - protein, 426 aa, Macrococcus bovicus
MAKRMPKVKGLDQTLTVLKEGYEFIPNRTRKLNTNVFEIRILGGKKAVVISGKEAAELFYDNDKTERAGTLPKRLVKTLFGKGAIHTTTGDKHKNRKSLFMSLMTDNNLEYLRKLTRNYWFEDTARLQAMDQTNVYQEATLVLLKIGFRFADVPFDTPERMEQYAKDMNAMVDSFGSIGTAYTGYRRALNARDRVEDYLEKQIIATRKGKLFPEEGSGLYEFSHWKDMNGKHMDSRLAAVDLMNTIRPLVAINRFISFGVLAMHEYPGERARVAQNDNSYAYKFAQEVRRYYPFVPFLPGKAKQNITFEGYKIDKDTMLILDIFGTLHSESLWENAEQFYPERFENWDGSPFDLIPQGGGDYYTNHRCAGEWMTIIIMEETMKYFAREIQYDVPPQDFTVDRTKLPGQINSGMIIENIRNNFDRRR

SEQ ID NO: 23 - protein, 426 aa, Macrococcus hajekii
MAKRIPKVKGFDQTLNVIKEGYEFIPNRTKKFNSNVFEMRVLGGKRAIVISGKEAAELFYDNEKIERQGTLPKRIVKTLFGKGAIHTTTGAKHKDRKSLFMSLMTENNLEYLRKLTRNYWFEDTARLQSKEQVNVFEESTLVLLKVAFRFADVPFDTPERMEQFAKDMNSMVDSFGSIGTVYSGYKKARDARTRVEDYLEKQIKLTRKGKLFPEEGSGLYEFSHWKDMNGKQMDSRLAAIDLMNTIRPLVAINRFISFGVLAMHEFPGERVKVAEDDNSYAYKFVQEIRRYYPFVPFLPGKVKQNFTFDGYKFDKETMVIMDIFGTLHSESLWNNAEQFYPERFENWDGSPFDLIPQGGGDYYTNHRCAGEWMTIIIMEETMKYFAREIQYDVPAQDFTVNRQKLPGQVKSGMIIENIRNNFDRRR

SEQ ID NO: 24 - protein, 426 aa, Macrococcus sp.
MSKSIPKEQGLDQTLNILKEGYEFIPNRTKKFNSNVFEIRVLGGKRAIVISGKEAAELFYDNEKIERAGTLPKRLVNTLFGKGAIHTTTGATHKDRKSLFMSLMTENNLEYLRKLTRNYWYTDTERLQMKEQTNVYLESTLVLLKIGFRFADVPFDTPERMEQYAKDMNAMVNSFGSIGTVYSGYKKALDARSRVEDYLEKQIKLTRKGKLNPEAGSGLYEFSHWKDMNGNHMDARLAAVDLMNTIRPLVAINRFISFGVLAMHEYPGERAKVAKDENSYAYKFAQEVRRYYPFVPFLPGKAKTDFTFEGYKFDKETMIILDIFGTLHSESLWNNAEQFYPERFEHWDGSPFDLIPQGGGDYYTNHRCAGEWMTIIIMEETMKYFAREIQYDVPAQDFTVDRTKLPGQVKSGMIIENIRNNFDRRQ

SEQ ID NO: 25 - protein, 423 aa, Salinicoccus luteus
MSTIKKHKGLDNTLKVMKEGYLYTTNQRRRLGSDNIFETRVLGGKRAIVISGKKAAELFYDNDKTERSGTLPKRLVNTLFGKGAIHTTTGKKHVDRKALFMSLMTEGNLKHLRELTRNHWYMNTHRMEQMDQINIYRESIILLTKVGTKWAGVQAPEEKIEEIATDMDIMIDSFKGLGTAYKGYKASVAARRRVEDWLEDQIIQTRKGKIFPPEGTALYEFAHWEDYKGNPMDSRLCAIDLMNTFRPLIAINRFVSFGLLAMYEHPISKEKIKNEPDYAYMFSQEVRRFYPFVPFLPGKAKTDIQFEGHDIEKDTMLVLDIYGTMHRDDVFENADEFYPERFRDWDGSPFDLIPQGGGDYYTNHRCAGEWMTIIIMEETMKYFAGKISYDVPEQDLTVDLNSIPGYIKSGFIIENVQENVDRG

SEQ ID NO: 26 - protein, 423 aa, Salinicoccus roseus
MSTIKKHKGLDNTLKVMKEGYLYTTNQRRRLGAENIFETRVLGGKRALVISGKKAAELFYDNDKTERSGTLPKRLVNTLFGKGAIHTTTGKKHIDRKALFMSLMTEGNLKHLRELTRNHWYMNTHRMEQMDQINIYRESIILLTKVGTKWAGVQAPEEKIEEIATDMDIMIDSFRGLGTAFKGYKESVAARRRVEDWLEDQIIQTRKGKIFPPEGTALYEFAHWEDYKGNPMDSRLCAIDLMNTFRPLIAINRFVSFGLLAMHEHPISKEKINNDPDYAYMFSQEVRRFYPFVPFLPGKAKTDIQFEGHDIEKDTMLVLDIYGTMHRDDVFENADEFHPERFLDWDGSPFDLIPQGGGDYYTNHRCAGEWMTIIIMEETMKYFAGKITYDVPEQDLTVDLNSIPGYIKSGFIIENVAEKVDRK

SEQ ID NO: 27 - protein, 423 aa, Salinicoccus roseus
MSTIKKHKGLDNTLKVMKEGYLYTTNQRRRLGAENIFETRVLGGKRALVISGKKAAELFYDNDKTERSGTLPKRLVNTLFGKGAIHTTTGKKHIDRKALFMSLMTEGNLKHLRELTRNHWYMNTHRMEQMDQINIYRESIILLTKVGTKWAGVQAPEEKIEEIATDMDIMIDSFRGLGTAFKGYKASVAARRRVEDWLEDQIIQTRKGKIFPPEGTALYEFAHWEDYKGNPMDSRLCAIDLMNTFRPLIAINRFVSFGLLAMHEHPISKEKINNDPDYAYMFSQEVRRFYPFVPFLPGKAKTDIQFEGHDIEKDTMMVLDIYGTMHRDDVFENADEFYPERFLDWDGSPFDLIPQGGGDYYTNHRCAGEWMTIIIMEETMKYFAGKITYDVPEQDLTVDLNSIPGYIKSGFIIENVAEKVDRK

SEQ ID NO: 28 - protein, 423 aa, Salinicoccus albus
MSTIKRDTGLDNTLKVMKQGYLYTENQRQRLGSDNIFETRVLGGKRALVISGKRAAELFYDNDKIERAGTLPKRLVNTLFGKGAIHTTTGKQHIDRKALFMSLMTEGNLKHLRELTRNHWYMNTHRMEQMDQINIYRESIILLTKVGTKWAGVQAPEEKVEEIATDMDIMIDSFRGLGTAFKGYKKSKDARRRVEDWLEDQIIQTRKGKIFPPEGTALYEFAHWEDYKGNPMDSRLCAIDLMNTFRPLIAINRFVSFGLLAMHEHPISKEKIKSDDDYAYMFSQEVRRYYPFVPFLPGKAKTDIEFDGHAIEKDTMMVIDIYGTMHRDDVFENANEFYPERFKEWDGSPFDLIPQGGGDYYTNHRCAGEWMTIIIMEETMKYFADRISYDVPQQDLTVDLNSIPGYIKSGFVIENVSENVDRN

SEQ ID NO: 29 - protein, 435 aa, Jeotgalibacillus alimentarius
MDVKKPVPRAKGIDNSLNFIKEGFHFLPNQRQELGSDIFETRLLGKKAVCIAGVEASEMFYDTEKFKREGAMPKPFKMSLFGEGSLHGMDGEAHLNRKRTLLSMFTPDRVEDLKRMAIKHLDEKVSEWEHADDVVIFDEMQEILARAGCEWAGVPLEEEEVKQRTREMIDMIDSFGGSLSRFKDGKKARNSHEAWLQDIIKGVRKGKMSPKPNTAAYIMSHHRENNGKRFDKEIAAVELSNAFRPLLATAYFLTFGVLAMHEHPETVEKLKNNAKYSHMFAQETRRYYPFVPAMVAKVKKNFTFKGYKFKKDTLVVLDIFGTNRHPDSWENADTFIPERFDGWKGSPFSFIPQGGGDHHTGHRCAGEWLTVIVMSSFFDFFAKNLTYDVPEQDLSFSMERMPTFPNSRLVIKNVRKTDDYEKNLAELMDGFSAIR

SEQ ID NO: 30 - protein, 436 aa, Jeotgalibacillus campisalis
MKVKTPIPRAKGIDNSLSLIKEGFHFLPNQRKEMQSDIFKTRILGQKAICIAGEEAAEVFYNNDLFKRKGAMPKPLKLSLFGEGSLHGLDGEPHKNRKRMFLSMFTPERVEDLVDLALKHLDAKAGEWQSQDGVAVLDQMQEILTRAGCEWAGVPLAEGEVQQRTDEVVAMVDSFGGSLSRFRDGKKARDSHEEWLQKIIKSVRKGKYNPPENTAVYTIAHHRDEKGKPFDLHTAAVELSNAFRPLIATANFLVFGALAMHEHPETRQKIAADEDRYSHMFSQEVRRYYPFVPAMAAKVKKDFTWHDYTFKKDTLVILDIFGTNRHPDSWEQAETFLPERFKDWQGSPFAFIPQGGGDHHIGHRCAGEWLTVYVMRAFFKYFAQNISYDVPEQDLSYSLERMPTFPKSGFVITNVKKAADSDELLHSMQKRHTAIQ

SEQ ID NO: 31 - protein, 435 aa, Jeotgalibacillus malaysiensis
MDVKKPVPRSKGIDNSLSLIKEGFYFLPNQRQKLGSDIFETRLLGKKAICMAGEEASELFYDTEKFKREDAMPKPLKMSLFGDGSLHGMDGEAHRNRKRSFLSMFTPDRVEDLKRLALEHLDAKVSHWEKADEVVIFDEMQEILARAGCEWAGVPLEENEVQQRTQEMIDMIDSFGGSLSRFKDGKKARDSHEAWLQEIIKGVRKGKMSPPPHTAAYVMAHHRENNGKRIDKGTAAVELSNSFRPLLATAYFLTFGIVAMHEHPLTVSKLQHHPRYSHMFAQETRRFYPFVPAMAAKVKKNFTFKGYKFKKDTLVVLDIFGTNRHPDSWENADTFIPERFEGWKGSPFSFIPQGGGDHHTGHRCAGEWLTVIVMSSFFEFFAKNLTYDVPEQDLSYSMERMPTFPNSRMIIQNVRKIADYEKNLNELMDGFSAIR

SEQ ID NO: 32 - protein, 436 aa, Jeotgalibacillus proteolyticus
MKVKTPIPRAKGIDNSLSLIKEGFHFLPNQRKEVESDIFKTRILGQKAICMCGEEAAEVFYDPDKFKRKGALPKPLKMSLFGDGSLHALDGEDHRNRKRMFLSMFTPERVEDLKKIALEHLAAKVDVWEKSDQVIFFGEMQEILTRAGCEWAGFPLEEEEVHQRTEEIVAMIDSFGGSLSRFRDGKKARDSHEEWLEGIIKAVRKGKMNPPAYTAAYIVAHHRENNGKRLDVRTAAIELSNAFRPLIATAYFLTFGAAAMHEHPETAHKLMENKDHYSHMFSQEVRRYYPFVPAMAAKVKEDFVWQDHKFKKNTLVILDIFGTNRHPDLWSDADSFIPERFRDWKGSPFSFIPQGGGDHHIGHRCAGEWLTVYVMRSFFQFLAQNITYDVPEQDLSYSLERMPTFPVSGLVITNVRKQADFNEKLNELDKSYTAIR

SEQ ID NO: 33 - protein, 434 aa, Halobacillus salinus
MSKPIPKAEGADNTLSLIQEGFPFFIKRHKELGTNIFETRLIGRKAICIVGEEASKIFYDTEKFKREGAMPKPLKMSLFGEGSLHGMDGEKHLHRKRTFLSMFTPDRVEDLKQNALEELDRKMEQWQERDKVVLYEEFPEILTRAVCKWAGVPLEEHEVRQRTDEMMSMVESFGGSLSRFQEGKKSRDSHEAWLKQIIKDVRKGKLQPEPNTAAYIMAHHRETDGKLWDVQTAAVELSNSFRPLLATVHLLVLGAVAMHEHPETRTRLEKGEPLYSHWFAQETRRFYPFVPAMIAKVKTPFLWEGYEFKKNTRVVLDLYGTNHHADSWVHPDAFIPERFENWQGSPFSFVPQGGGDHHTGHRCAGEWLTVIVMSSFFEFLAKNVSYDVPEQNLSFSLERMPTRPESGFVIENVRKKDAYEETFDELNQGFAARR

SEQ ID NO: 34 - protein, 436 aa, Bacillus clarkii
MKVKTPIPKAKGIDNSMSLIKEGFHFLPNRREELGSDIFETRLMGRKAVCMSGEEAAEVFYDNDKFKRKGALPKPLQLSLFGTGSVHALDGEAHKNRKRMFLSMFTPERVEDLNRIALKHLDNKVNQWVKRDKVILIDEMHEILTRAGCEWAGVPLKEEEVEQRTAEVGQMIDSFGGSLSRWRNGKKARDSHEEWLEGIIKKIRKGKMEVPANTAAYIVAHHRELNGKRLDSRQAAIELSNSFRPLIATAYFLTLGAVAMHEHPGTMEKLKKDEGNYSHMFSQEVRRYYPFVPAMAAKVKKDFVWHDYKFKKDTLVVLDIFGTNRHPDNWEQADEFIPERFKDWKGSPFSFIPQGGGDHHIGHRCAGEWLTVMVMRSFFKYFVNNMNYHVPEQNLAYSLERMPTFPKSGFVITDVKKASDSGEKMQEMMHNPITVK

SEQ ID NO: 35 - protein, 436 aa, Bacillus polygoni
MKVKTPIPKAKGIDNSMSLIKEGFHFLPNRREELGSDIFETRLMGRKAVCMSGEEAAEVFYDNDKFKRKGALPKPLQLSLFGTGSVHALDGEAHKNRKRMFLSMFTPERVEDLNRIALKHLDNKVNQWVKRDKVILIDEMHEILTRAGCEWAGVPLKEEEVEQRTAEVGQMIDSFGGSLSRWRNGKKARDSHEEWLEGIIKKIRKGKMEVPANTAAYIVAHHRELNGKRLDSRQAAIELSNSFRPLIATAYFLTLGAVAMHEHPGTMEKLKKDEGNYSHMFSQEVRRYYPFVPAMAAKVKKDFVWHDYKFKKDTLVVLDIFGTNRHPDNWEQADEFIPERFKDWKGSPFSFIPQGGGDHHIGHRCAGEWLTVMVMRSFFKYFVNNMTYHVPEQNLAYSLERMPTFPKSGFVITDVKKAADSGEKMQEMMHNPITVK

SEQ ID NO: 36 - protein, 434 aa, Halobacillus salinus
MSKPIPKAEGADNTRSLIQEGFPFFIKRHKELGTNIFETRLIGRKAICIVGEEASKIFYDTEKFKREGAMPKPLKMSLFGEGSLHGMDGEKHLHRKRTFLSMFTPDRVEDLKQNALEELDRKVEQWQERDKVVLYEEFPEILTRAVCKWAGVPLEEHEVRQRTDEMMSMVESFGGSLSRFQEGKKSRDSHEAWLKQIIKDVRKGKLQPEPNTAAYIMAHHRETDGKLWDVQTAAVELSNSFRPLLATVHLLVLGAVAMHEHPETRTRLEKGEPLYSHWFAQETRRFYPFVPAMIAKVKTPFLWEGYEFKKNTRVVLDLYGTNHHADSWVHPDAFIPERFENWQGSPFSFVPQGGGDHHTGHRCAGEWLTVIVMSSFFEFLAKNVSYDVSEQNLSFSLERMPTRPESGFVIENVRKKDAYEETFDELNQGFAARR

SEQ ID NO: 37 - protein, 435 aa, Halobacillus sp.
MIHKDIPRAKGIDNTMNLIKEGFHFLPNQRQALNSDVFETRLIGKKAICIAGEEASKLFYDTEKFKREGAMPKPLKMSLFGDGSLHGLDGEEHRNRKRTFLSMFTPDRVEDLKKLALKHLDAKATEWQEQEEVYLFDEMQEILARAGCEWAGVPLQEEEVKQRTDEMIEMIDSFGGSLSRFREGKQARNHHEKWLGQIIKDVRKGKLDPEPNTAAYIMAHHRETNGKKWDTRTAAVELSNSFRPLLATAYFLTFGAVAMHEHPESKEKLKQDTSRYSHWFAQETRRYYPFVPAMVAKVKEDFIWNDFLFKKNTLVVLDIFGTNRHPDSWEEPDAFIPERFKDWEGSPFSFIPQGGGDHHTGHRCAGEWLTVIVMSSFFEFFAKNLSYDVPEQDLSFSMERMPSYPNSRFKIQNVKKTGYYDEHLSDLNRGFSAIR

SEQ ID NO: 38 - protein, 438 aa, Planomicrobium flavidum
MRIRKPIPKAEGLDNTLALIKEGFHFIPSRREKLASDIFETRLIGRKAVCMAGEEAAAIFYDNNLFKRAGAAPKPLKQGLLGVGGIHGRDGEDHHQQKRMFLSMMTPERLEDMRRIAVEELDRKAAAWQKEDSVAMLPEMEEIMGLAGMRWAGLPLGSEQEAKRRAKELADMVDSFGGSFSRYQEGKRARESHEEWLRNIIRDIRQEKYNPPPYTAAYIVAHHRGADGKLLDLETAAVDLNNSYRPLVATAYFIVFGMLAMYEHPETVAKIRSNEGNYSHMFSQEIRRYYPFAPAMVAKAKKDFIWHGYHFKKDQTVVLDLFGTNRHPDNWEEADRFIPERFHNWSGSPFAFVPQGGGDHYMGHRCAGEWMTVIVMQAFFHYLTAGVAYDVPEQDLTYDLRRMPTMPKSGFVIRNVRRSGNYPLDALMEDQNHTAVKF

SEQ ID NO: 39 - protein, 422 aa, Staphylococcus delphini
MAKKLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDKVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPYLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 40 - protein, 422 aa, Staphylococcus delphini
MAKKLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 41 - protein, 422 aa, Staphylococcus delphini
MAKKLPKDTGLDNTLKIINDAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPHLCAVDLMNIIRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 42 - protein, 423 aa, Salinicoccus sp. CT19
MSTIKRDKGLDNTLKVMKEGYLYTTNQRNRLGADNIFETRGLGGKRVLVLSGKKAAETFYDNDKIERKGTLPKRVVNTLFGKGAIHTTTGKKHIDRKALFMSLMTEGNLKHLRELTRNHWYMNTHRMEQMDQINIYRESIILLTKVGTKWAGVQAPEEKIEEIATDMDIMIDSFRGLGTAFKGYRSSVKARRRVEDWLEDQIIQTRKGKIFPPEGTALYEFAHWEDYLGNPMDSRLCAIDLMNTFRPLIAINRFVSFGLLAMHEFPVSREKINNEDDYAYMFSQEVRRFYPFVPFLPAKAKTDFHFDGHKVDKDTMLLLDIYGTMHREDVFENPNEFRPERFLDWDGSPFDLIPQGGGDYYTNHRCAGEWMTVIIMEETMKYFAGRITYDVPEQDLRVDLNSIPGYVKSGFVIENVKENLDRR

SEQ ID NO: 43 - protein, 422 aa, Staphylococcus delphini
MAKKLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPHLCAVDLMNIVRPLVAVNRFVSYGVKALIEFDQERKKLQVTNDPNYAYKFAQEVRRIFPFVPFVPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 44 - protein, 422 aa, Staphylococcus delphini
MAKKLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVRAGQQYAEPGTALYEFAHWKDLNDQPMDPHLCAVDLMNIVRPLVAVNRFVSYGVKALIEFDQERKKLQVTHDPNYAYKFAQEVRRIFPFVPFLPGRLKQTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 45 - protein, 422 aa, Staphylococcus delphini
MAKQLPKDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVRAGQQYAEPGTALYEFAHWKDLNDQPMDPHLCAVDLMNIVRPLVAVNRFVSYGVKALIEFDQERKKLQVTHDPNYAYKFAQEVRRIFPFVPFLPGRLKKTVEFDGFKLKKGTLTVLDIFGTTHDPELFKNPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 46 - protein, 422 aa, Staphylococcus delphini
MAKKLPKDTGLDNTLKIINEAYTYVPKRLENFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVREGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 47 - protein, 422 aa, Staphylococcus delphini
MAKKLPRDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNLMIDSFSGLGQSLKGYREAKKARARVEQFLQDQIEAVRAGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKQTVEFDGFKLKKGTLTVLDIFGTTHDPELFENPYQFNPDRFDNWDGSPFDLIPQGGGDFYTNHRCAGEWMTVIVMEETIQYFANKIDFEAPVQDLSVKLDQFPGKVTSGTIIKSVYPRI

SEQ ID NO: 48 - protein, 422 aa, Staphylococcus cornubiensis
MAKKLPKDTGLDNTLKIINEAYTYVPKRLEQFGTKAFETRALGMKPVVVISGKEAAKLFYDNDKISRKGTLPKRIVHTLFGKGAIHTTEGKVHVDRKALFMSLMTEKNLKYLRELTRNYWFMNTERMQNMDEVNVYQEAGLILTQVGFRWAGLKQTDEEAAKNAEDMNIMIDSFSGLGQSLKGYREAKKARARVEQFLQDQIEAVRAGEQYAEPGTALYEFAHWKDLNDQPMDPHLCVVDLMNIVRPLVAVNRFVSYGVKALIEFDQERKKLQVTNDANYAYKFAQEVRRIFPFVPFLPGRLTKTVEFEGFKIKKDTLVALDIFGTTHDPELFENPYQFNPDRFENWDGSPFDLIPQGGGDFYTNHRCAGEWMTVIVMEETMQYFANKIDFDAPAQDLSVKLDQFPGKVTSGTIIKNVYPRI

SEQ ID NO: 49 - protein, 422 aa, Staphylococcus delphini
MAKKLPRDTGLDNTLKIINEAYTYVPKRLEKFGTKAFETRALGMKPVVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQDQIEAVRAGQQYAEPGTALYEFAHWKDLNDQPMDPHLCAVDLMNIVRPLVAVNRFVSYGVKALIEFDQERKKLQVTHDPNYAYKFAQEVRRIFPFVPFLPGRLKQTVEFDGFKLKKGTLTVLDIFGTTHDPELFENPYQFNPDRFDNWDGSPFDLIPQGGGDFYTNHRCAGEWMTVIVMEETIQYFANKIDFEAPAQDLSVKLDQFPGKVTSGTIIKSVYPRI

SEQ ID NO: 50 - protein, 422 aa, Staphylococcus intermedius
MAKKLPKDTGLDNTFKIINEAYTYVPKRLEQFGTKAFETRALGMKPVVVISGKAAAKLFYDNDKISRKGTLPKRIVHTLFGKGAIHTTEGKVHVDRKALFMSLMTEKNLKYLRELTRNYWFMHTERMQNMDEVNVYKEAGLILTQVGFRWAGLKQTDEEAAKNAEDMNIMIDSFSGLGQSLKGYRQAKKARARVEQFLQDQIEAVRAGKQYAEPGTALYEFAHWKDLNNQPMDSHLCAVDLMNIVRPLVAVNRFVSYGVKALIEFDQERKKLQVTNDPNYAYKFAQEVRRIFPFVPFLPGRLTKTVEFDGFKIKKGTLVALDIFGTTHDPELFENPYQFNPDRFENWDGSPFDLIPQGGGDFYTNHRCAGEWMTVIVMEETIQYFANKIDFDAPAQDLSVKLDQFPGKVTSGTVIKNVYPRI

SEQ ID NO: 51 - protein, 423 aa, Salinicoccus roseus
MSTIKKHKGLDNTLKVMKEGYLYTTNQRRRLGAENIFETRALGGKRVLVISGKKAAELFYDNDKTERSGTLPKRLVNTLFGKGAIHTTTGKKHIDRKALFMSLMTEGNLKHLRELTRNHWYMNTHRMEQMDQINIYRESIILLTKVGTKWAGVQAPEEKIEEIATDMDIMIDSFKGLGTAFKGYKESVAARRRVEDWLEDQIIQTRKGKIFPPEGTALYEFAHWEDYKGNPMDSRLCAIDLMNTFRPLIAINRFVSFGLLAMHEYPISKEKINNDPDYAYMFSQEVRRFYPFVPFLPGKAKTDIQFEGHDIEKDTMLVLDIYGTMHQDDVFENADEFYPERFLDWDGSPFDLIPQGGGDYYTNHRCAGEWMTIIIMEETMKYFAGKITYDVPEQDLTVDLNSIPGYIKSGFIIENVAEKVDRK

SEQ ID NO: 52 - protein, 423 aa, Salinicoccus halodurans
MGTIKKDKGLDNTLKVMKQGYLYTANQRQRLGADNIFETRALGGKRVVVINGKEAAELFYDNDKTERSGTLPKRLVNTLFGKGAIHTTTGKKHIDRKALFMSLMTENNLQHLRELTRNHWYMNTHKMEQMDQINIYRESIVQLTKIGTKWAGVQAPEEKIEDIATDMDIMIDSFKGLGSAFKGYKESVNARKRVEDWLEEQIIETRKGKIFPPEGTALYEFAHWEDYQGNPMDSRLCAIDLMNTFRPLIAINRFVSFGLLAMHEHPISKEKIKTDDDYAYMFSQEVRRFYPFVPFLPGKAKTDIEFKGHKIEKDTMMVIDIYGTMHDEKVFENPDEFYPERFKEWDGSPFDLIPQGGGDYYTNHRCAGEWMTIIIMEETMKYFAERITYDVPEQDLTVDLNSIPGYVNSGFIIENVQENIDRK

SEQ ID NO: 53 - protein, 424 aa, Salinicoccus kekensis
MATIKKDKGIDNTAKIMKQGYLYTTNQRERLGVKDGVFETRALGGKRVIILSGKDGAELFYDNDKIERSGTLPKRVVNTLFGKGAIHTTTGKVHIDRKALFMSLMTEGNLQYLRELTRNHWQMNTQRMENMDQVNVYRESIILLTKVGTRWAGVQAPEKEIENIATDMDIMIDSFKAIGSAFKGYRASKAARRRVEDWLEDQIIQTRKGKIHPPEGTALYEFAHWKDYKGEPMDSRLCGIDLMNTFRPLIAINRFIAFGALAMYENPVAREKIKQDDDYAYMFAQEVRRFYPFVPFLPGKVKEDFQYKGYDFEKDTMLAIDIYGTMHDPTVWENPNEFYPERFKDWDGSPFDLIPQGGGDYYTNHRCAGEWMTVIIMQETMKYFASRITYDVPEQDLTVDLNSLPGYVKSGFIIENVREVVDRG

SEQ ID NO: 54 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLERQIKAVREGKQFAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVASGTVIRNVRPRR

SEQ ID NO: 55 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLERQIKAVREGKQFAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 56 - protein, 421 aa, Staphylococcus felis
MNKIPKDKGLDNTLKIVKEAYTYVPRRLEAFDTNIFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 57 - protein, 421 aa, Staphylococcus felis
MNKIPKDKGLDNTLKIVKEAYTYVPRRLEAFDTNIFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQFAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 58 - protein, 421 aa, Staphylococcus felis
MNKIPKDKGLDNTLKIVKEAYTYVPRRLEAFDTNIFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRLRR

SEQ ID NO: 59 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMNEVNVYEEATLLLTKIGFRWAGLKQTHEEAVQNAKDMDLLIDSFSALGQSPAGYKKAKKARERVEKFLEHQIKAVREGEQFAAPNTALYEFAHWKDLNDKPMDIHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 60 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 61 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEVAVQNAKDMDLLIDSFSALGQSPAGYKKAKKARERVEKFLEHQIKAVREGEQYAAPNTALYEFAHWKDLNDKPMDLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDSKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 62 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQLMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMNLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 63 - protein, 421 aa, Staphylococcus felis
MNKIPKDKGLDNTLKIVKEAYTYVPRRLEAFDTNVFETRALGMKKVLVVSGKKAAELFYDNEKISRKGALPKRIVNTLFGQGAIHTTEGKQHVDRKALFMSLMTTKNLKYLRELTRNYWFAHTQRMEDMDEVNVYEEATLLLTKIGFRWAGLKQTHEEAVQNAKDMDLLIDSFSALGQSPAGYKKAKKARERVEKFLEHQIKAVREGEQYAAPNTALYEFAHWKDLNDKPMNLHLCAVDLMNVVRPLVAVNRFVSYAVKALIEFDQERLKLQVTKDQNYAYKFAQEVRRIFPFVPFLPGRLKQNIEFDGYSLKKNTLIILDVFGTTHDPKLFENPYQFNPNRFEDWDGSPFDLIPQGGGDFYTNHRCAGEWMTVIVMEETINYFANKIDFSVPTQDLSVKLDQFPGKVTSGTVIRNVRPRR

SEQ ID NO: 64 - protein, 424 aa, Fictibacillus enclensis
MSKQLNIPKEEGLDHSLALIQEGYQYITSRRTKFKSDLFETRLLGQKVVCIGGQEGAELFYDNEKFERKGAVPKRIQKSLFGENAIHTLDGEAHEHRKLLFMSLMTEERLELLAELTREEWRLAAAEWAKMEQVVLFDEVQQILCRVACRWAGVPIDAIEVKPLARDFIAMVDAFGAVGPRHQKGRKARKKMEDYFKGLIIEIRHRRIEPIEGTALHKIAWHRDHKEKHLDEHTAAVELLNVLRPIVAITYLVTFGALALFEHNVWKEKLQSGDEESVQMFAQEVRRFYPFAPFLGTKVKNGFEWKGHTFEKGQLVLLDVYGTNHDNRLWEEPYVFNPERFRNWNGGMFDLIPQGGGDEYTGHRCPGEMATIQVMKTSFSFLTNEMIYNVPVGQDLSYSLSRMPTYPESGFIIQGVSIKSQTTV

SEQ ID NO: 65 - protein, 424 aa, Fictibacillus sp. S7
MSKQLNIPKEEGLDHSLALIQEGYQYITSRRTKFKSDLFETRLLGQKVVCIGGQEGAELFYDNEKFERKGAVPKRIQKSLFGENAIHTLDGEAHEHRKLLFMSLMTEERLELLAELTREEWRLAAAEWAKMEQVVLFDEVQQILCRVACRWAGVPIDAIEVKPLARDFIAMVDAFGAVGPRHQKGRKARKKMEDYFKGLIIEIRHRRIEPIEGTALHKIAWHRDHKEKHLDEHTAAVELLNVLRPIVAITYLVTFGALALFEHNVWKEKLQSGDEESVQMFAQEVRRFYPFAPFLGAKVKNGFEWKGHTFEKGQLVLLDVYGTNHDNRLWEEPYVFNPERFRNWNGGMFDLIPQGGGDEYTGHRCPGEMATIQVMKTSFSFLTNEMIYNVPVGQDLSYSLSRMPTYPESGFIIQGVSIKSQTTV

SEQ ID NO: 66 - protein, 420 aa, Lentibacillus salicampi
MKGVAPVAIPRENVLDNTLTLLREGYHYIPGRRRHFKSDIFQTRLLGQKVICIGGKEAAEVFYDEDKFTRIGAIPNRMKESLFGKNGVHVLEGNAHSHRKTMFMSLMTSERLSELTALTEKQWEVAIADWQQKRRVILFPEAEKIMCRIACEWAGVPLWANELSQRTRDLSAMIDAFGAIGPRHWQGRIARNRSERWIRRIIAQVRSGELKPREETALYTFAWHRDLQDRLLDLQTAAVELLNIVRPIVAVGRFVTFGALAINDFPETKEKLRVGDDDYIRMFVQEVRRYYPFGPFLGAKVRSNFKWTGHQFKKGTLVLLDIYGTNHDPDLWDKPDEFRPERFDEWEGSPFDFIPQGGGYDMGHRCPGEWITIDIMKVSLAFLANRIDYNVPKQNLDYSMVRMPSIPKSRFIMRDVAISS

SEQ ID NO: 67 - protein, 426 aa, Staphylococcus vitulinus
MGKQIPKDRGLDSTLKVLKEGYKYVPNRLEKFDTNIFELRALGGRRTVVISGKEAAELFYNNELTERQGTLPKRVVNTLFGKGAIHTTGGKKHIDRKALFMSLMTEENLDYLRELTRSTWFMNTERMERMDEVNVYKESIILLTKVGFRWAGIIASPEEIESCAQDMDTMIDSFKNIGTVFKGYREAKQARARVETFLENQIIAVREGKLTPPQGTALHEFSHWEDFEGNLMDSRLCAIDLMNVVRPLVAINRFISFGVKALHDYPGEAEKVFNNEDDYAYKFVQEIRRFYPFVPFLPGKAAVDIEFEGYTIEKDTFLVLDIYGTLHREGLWENPERFYPNRFSDWDGSPFDLIPQGGGDYHTNHRCAGEWMTLIIMEESMKYFARNISYDVKKDQDLSVNLNKLPGRVVSGTIIENVNALVNRNV

SEQ ID NO: 68 - protein, 429 aa, Staphylococcus sciuri
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWENPERFYPNRFSDWDGSPFDLIPQGGGDYYTNHRCAGEWMTIIIMEESMKYFARNISYDVKKDQDLSVNLNKLPGRVVSGTIIENVNALVNRNVESV

SEQ ID NO: 69 - protein, 429 aa, Staphylococcus sciuri
MGKQIPKDRGLDSTLKVLKEGYKYVPNRLEKFDTNIFELRALGGRRTVVFSGKEAAEIFYNNELIERQGTLPKRVVNTLFGKGAIHTTGGKKHIDRKALFMSLMTEENLEYLRELTRSTWFMNTERMERMDEVNVYKESIILLTKVGFRWAGIIASPEEIESCAKDMDTMIDSFKNIGTAFKGYREAKKARDRVETFLENQIIAVREGKLTPPQGTALHEFSHWEDFEGNLMDSRLCAIDLMNVVRPLVAINRFVSFGVKALHDYPGEAEKVFNNENDYAYKFVQEVRRFYPFVPFLPGKAAVDIEFDGYKIEKDTFLVLDIYGTLHREGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 70 - protein, 429 aa, Staphylococcus sciuri
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVNIEFDGYTIEKDTFLVLDIYGTLHRDGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 71 - protein, 429 aa, Staphylococcus sp.
MGKQIPKDRGLDSTLKVLKEGYKYVPNRLEKFDTNIFELRALGGRRTVVFSGKEAAEIFYNNELIERQGTLPKRVVNTLFGKGAIHTTGGKKHIDRKALFMSLMTEENLEYLRELTRSTWFMNTERMERMDEVNVYKESIILLTKVGFRWAGIIASPEEIESCAKDMDTMIDSFKNIGTAFKGYREAKKARDRVETFLENQIIAVREGKLTPPQGTALHEFSHWEDFEGNLMDSRLCAIDLMNVVRPLVAINRFVSFGVKALHDYPGEAEKVFNNENDYAYKFVQEVRRFYPFVPFLPGKAAVDIEFDGYTIEKDTFLVLDIYGTLHREGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 72 - protein, 429 aa, Staphylococcus sciuri
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWDNPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 73 - protein, 429 aa, Staphylococcus sciuri
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCARDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 74 - protein, 429 aa, Staphylococcus sp.
MGKQIPKDRGLDSTFKVLKEGYKYVPNRLEKFDTNIFELRALGGRRTVVISGKEAAELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 75 - protein, 429 aa, Staphylococcus vitulinus
MGKQIPKDRGLDSTLKVLKEGYKYVPNRLEKFDTNIFELRALGGRRTVVISGKEAAELFYNNELTERQGTLPKRVVNTLFGKGAIHTTGGKKHIDRKALFMSLMTEENLDYLRELTRSTWFMNTERMERMDEVNVYKESIILLTKVGFRWAGIIASPEEIESCAHDMDTMIDSFKNIGTVFKGYREAKQARARVETFLENQIIAVREGKLTPPQGTALHEFSHWEDFEGNLMDSRLCAIDLMNVVRPLVAINRFISFGVKALHDYPGEAEKVFNNEDDYAYKFVQEIRRFYPFVPFLPGKAAVDIEFEGYTIEKDTFLVLDIYGTLHREGLWENPERFYPNRFSDWDGSPFDLIPQGGGDYHTNHRCAGEWMTLIIMEESMKYFARNISYDVKKDQDLSVNLNKLPGRVVSGTIIENVNALVNRNVESV

SEQ ID NO: 76 - protein, 429 aa, Staphylococcus vitulinus
MGKQIPKDRGLDSTLKVLKEGYKYVPNRLEKFDTNIFELRALGGRRTVVISGKEAAELFYNNELTERQGTLPKRVVNTLFGKGAIHTTGGKKHIDRKALFMSLMTEENLDYLRELTRSTWFMNTERMERMDEVNVYKESIILLTKVGFRWAGIIASPEEIESCAQDMDTMIDSFKNIGTVFKGYREAKQARARVETFLENQIIAVREGKLTPPQGTALHEFSHWEDFEGNLMDSRLCAIDLMNVVRPLVAINRFISFGVKALHDYPGEAEKVFNNEDDYAYKFVQEIRRFYPFVPFLPGKAAVDIEFEGYTIEKDTFLVLDIYGTLHREGLWENPERFYPNRFSDWDGSPFDLIPQGGGDYHTNHRCAGEWMTLIIMEESMKYFARNISYDVKKDQDLSVNLNKLPGRVVSGTIIENVNALVNRNVESV

SEQ ID NO: 77 - protein, 429 aa, Staphylococcus sp.
MGKQIPKDRGLDSTLKVLKEGYIYVPNRLEKFDTNIFELRALGGRRTVVISGKEAAELFYNNELTERQGTLPKRVVNTLFGKGAIHTTGGKKHIDRKALFMSLMTEENLDYLRELTRSTWFMNTERMERMDEVNVYKESIILLTKVGFRWAGIIASPEEIESCAHDMDTMIDSFKNIGTVFKGYREAKQARARVETFLENQIIAVREGKLTPPQGTALHEFSHWEDFEGNLMDSRLCAIDLMNVVRPLVAINRFISFGVKALHDYPGEAEKVFNNEDDYAYKFVQEIRRFYPFVPFLPGKAAVDIEFEGYTIEKDTFLVLDIYGTLHREGLWENPERFYPNRFSDWDGSPFDLIPQGGGDYHTNHRCAGEWMTLIIMEESMKYFARNISYDVKKDQDLSVNLNKLPGRVVSGTIIENVNALVNRNVESV

SEQ ID NO: 78 - protein, 429 aa, Staphylococcus sciuri
MGKQIPKDRGLDSTFKVLKEGYKYVPNRLEKFDTNIFELRALGGRRTVVISGKEATELFYNNDLIERQGTLPKRVVNTLFGKGAIHTTGGKKHIDRKALFMSLMTEENLEYLRELTRSTWFMNTERMERMDEVNVYKESIILLTKVGFRWAGVIASPEEIESCAQDMDTMIDSFKNIGTAFKGYREAKKARDRVETFLENQIIAVREGKLTPPQGTALHEFSHWEDFEGNLMDSRLCAIDLMNVVRPLVAINRFISFGVKALHDYPGEAEKVFNNENDYAYKFVQEVRRFYPFVPYLPGKAAVDIEFDGYTIEKDTFLVLDIYGTLHRDGLWENPERFYPNRFSDWDGSPFDLIPQGGGDYYTNHRCAGEWMTIIIMEESMKYFARNISYDMKKDQDLSVNLNKLPGRVVSGTIIENVNALVNRNVESV

SEQ ID NO: 79 - protein, 429 aa, Staphylococcus stepanovicii
MGKQIPKERGLDSTFKVLKEGYKYVPNRLEKHNSNIFELRALGGRRTVVFSGKEAAEVFYNNDLIERQGTLPKRVVNTLFGKGAIHTTGGKKHIDRKALFMSLMTEENLDYLRELTRSFWFTNIERMERMDEVNVYKESIILLTKVGFRWAGVIASPEEIERCAEDMDTMIDSFKNIGTAFKGYKEAKQARDRVETFLENQIIAVREGKLTPPQGTALYEFSHWEDFEGNPMDSRLCAIDLMNVVRPLVAINRFVSFGVKALHDYPGEAEKVFNNENDYAYKFVQEVRRFYPFVPFLPGRAAVDIEFEDYTIEKGTFLVLDIYGTLHKEGLWENPERFYPNRFSDWDGSPFDLIPQGGGDYNTNHRCAGEWMTIIIMEESMKFFAKDISYDVKKDQDQSVNLNKLPGRVASGMIIENVNALVNRNVESV

SEQ ID NO: 80 - protein, 429 aa, Staphylococcus sp.
MGKQIPKDRGLDSTFKVLKEGYNYVPSRLEKFDSNIFELRALGGRRTVVISGKEAAELFYNNELIERQGTLPKRVVNTLFGKGAIHTTGGKKHIDRKALFMSLMTEENLDYLRELTRSTWFMNTERMERMDEVNVYKESIVLLTKVGFRWAGIIASPEEIESCAKDMDTMIDSFKNIGTAFKGYKEAKQARARVETFLENQIIAVREGKLTPPQGTALHEFSHWEDFEGNLMDSRLCAIDLMNVVRPLVAINRFVSFGVKALHDYPGEAEKVFNNEDDYAYKFVQEVRRFYPFVPFLPGKAAVDIEFDGYTIEKDTFLVMDIYGTLHKEGLWENPERFYPNRFSDWDGSPFDLIPQGGGDYNTNHRCAGEWMTIIIMEESMKYFARNISYDMKQDQDLSVKLNKLPGRVVSGTIIENVNALVNRNVESV

SEQ ID NO: 81 - protein, 429 aa, Staphylococcus fleurettii
MGKQIPKDRGLDSTFKVLKEGYNYVPSRLEKFDSNIFELRALGGRRTVVISGKEAAELFYNNELIERQGTLPKRVVNTLFGKGAIHTTGGKKHIDRKALFMSLMTEENLDYLRELTRSTWFMNTERMERMDEVNVYKESIVLLTKVGFRWAGIIASPEEIESCAKDMDTMIDSFKNIGTAFKGYKEAKQARARVETFLENQIIAVREGKLTPPQGTALHEFSHWEDFEGNLMDSRLCAIDLMNVVRPLVAINRFVSFGVKALHDYPGEAEKVFNNEDDYAYKFVQEVRRFYPFVPFLPGKAAVDIEFDGYTIEKDTFLIMDIYGTLHKEGLWENPERFYPNRFSDWDGSPFDLIPQGGGDYNTNHRCAGEWMTIIIMEESMKYFARNISYDMKQDQDLSVKLNKLPGRVVSGTIIENVNALVNRNVESV

SEQ ID NO: 82 - protein, 425 aa, Abyssicoccus albus
MGKSIPRDRGMDNTVKALKKGYTYIPELRERLNSNDIVATRALGGKKTVIISGKDAAELFYDNDKIERQGTLPPRVVKTLFGKGAIHTTTGKTHIDRKALFMSLMTEENLSYLRELTRLEWYYNTTRMSHMDEVNVYKEAIYVLTKVGIKWAGLHASEDEINQYAEDMDTMIDSFKAIGTMFKGYKEARKARDRVETWLEAQIQGVRDGEITPPEGTALYEFAHWKDYEGNPMDARLAAIDLMNVIRPLVAINRFVAFGTLAMYEFPKERTKVAQNEEDYVYKFIQEVRRFYPFVPYLPGKAGKDIEFRGYEIKKDTFMVLDVYGTLHREDLWENADQFIPERFEDWDGSPFDLIPQGGGDYYTNHRCAGEWMTVIVMEESMKYFAQAIKYEVPAQDLTVDLNKLPGRVKSGFIIKNVERDINRF

SEQ ID NO: 83 - protein, 425 aa, Auricoccus indicus
MGKSIPRDRGMDNTVKALKKGYTYIPELRERLNSNDIVATRALGGKKTVIISGKDAAELFYDNDKIERQGTLPPRVVKTLFGKGAIHTTSGKTHIDRKALFMSLMTEENLSYLRELTRLEWYYNTTRMSHMDEVNVYKEAIYVLTKVGIKWAGLHASEDEINQYAEDMDTMIDSFKAIGTMFKGYKEARKARDRVETWLEAQIQGVRDGEITPPEGTALYEFAHWKDYEGNPMDARLAAIDLMNVIRPLVAINRFVAFGTLAMYEFPKERTKVAQNEEDYVYKFIQEVRRFYPFVPYLPGKAGKDIEFRGYEIKKDTFMVLDVYGTLHREDLWENADQFIPERFEDWDGSPFDLIPQGGGDYYTNHRCAGEWMTVIVMEESMKYFAQAIKYEVPAQDLTVDLNKLPGRVKSGFIIKNVERDINRF

SEQ ID NO: 84 - protein, 423 aa, Aliicoccus persicus
MSTIKKDKGLDNTLKAIKQGYKYTENQRKRLDSNNIFETRGLGGKRIIFISGKEAAETFYDNDKIEREGMLPKRVVSTLFGKGAIHTTTGKVHIDRKALFMSLMTEGNLQYLRELTRNQWRSNTINMERQDTVNVYREAIVQLTHIGTRWAGVQAPAEEIDRIATDMDLMIDSFKGLGLSFKGFRESKAARRRVEDWLEDQIHKTRKGVIHPPEGTALYEFAHWEDFEGNPMDSRLCAIDLMNTFRPLIAINRFVTFGVLALHQYPETREKIKNDPDYAYMFSQEVRRYYPFVPFLPAKAKVDFEFEGHKIDKDVMILLDVYGTMHRDDVFDNPNEFYPERFKDWDGSPFDLIPQGGGDYHTNHRCAGEWMTIIIMEETMKYFAERITYDVPEQDLTVDLEKLPGSVASGMNITNVRENVNRK

SEQ ID NO: 85 - protein, 423 aa, Staphylococcus lutrae
MNKKLPKDTGLDNTLKIINEAYTYVPKRLEKFGTKAFETRALGMKPITVMSGKAAAELFYDNDKVSRTGTLPKRVVHTLFGKGAIHTTEGKVHVNRKALFMSLMTEKNLKYLRELTRHYWFMHTERMQNMDEVSVYKEAGIILTKVGFRWAGLKQTDEEAVKNAEDMNKMIDSFSRLGQSVKGYRQAKKARARVEQFLRDQIKAVRENKQYAEPGTALYEFAHWKDLKGQPMDLHLCAVDLMNIVRPLVAVNRFISYAVKALIEFDQERQKLQVTHDPNYAYKFAQEVRRIFPFVPFLPGKLKKTIDFDGFKIKKGTMVALDIFGTTHDPELFKNPYQFNPNRFEKWDGSPFDLIPQGGGDFYTNHRCAGEWMTIIVMEETVQYFANKIDFEVPAQDLSVKLDRFPGKVTSGTLIKNVRPRRS

SEQ ID NO: 86 - protein, 425 aa, Staphylococcus aureus
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTTEEAEQNAKDMDIMIDSFKGLGQTIGGGYRKAKKARARVEQFLEKQIIAVRKGKISAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPKLFENPYQFNPDRFENWDGSPFDLIPQGGGDFYTNHRCAGEWMTIIVMEETIKYFANKIDFDAPSQDLSVKLDQFPGKVTSGTIIQNVRPRVQR

SEQ ID NO: 87 - protein, 425 aa, Staphylococcus agnetis
MAKQLPKDPGLDNTFKVLKEAYTYVPCRLEKFNSKAFQTTGMGMKPIAVISGKDAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTAEEAEQNAKDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 88 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTAEEAEQNAKDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 89 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQREVNIYKESIYVLTKIGFRWAGIHQTAEEAEQNAKDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 90 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTTEEAEQNAKDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 91 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESVYVLTKIGFRWAGIHQTAEEAEQNAKDMDIMIDSFKGLGQTIGGGYRKAKKARARVEQFLENQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 92 - protein, 423 aa, Salinicoccus sediminis
MGTIKRDKGLDNTLKVMKQGYLYITNQRKRLGSENIFETRALGGKKIIVISGKDAAELFYDNDKIERSGTLPKRVVNTLFGKGAIHTTTGKKHIDRKALFMSLMTEGNLKHLRELTRNHWYMNTQKMEQMDQINIYRESIVQLTKIGTKWAGVQAPEEKIEEIATDMDVMIDSFKGLGGKFKGYKASVDARKRVEDWLEDQIIQTRKGKIFPPEGTALYEFAHWEDYKGNPMDSRLCAIDLMNTFRPLIAINRFVSFGLLAMHEHPITREKIKSDDDYAYMFSQEVRRFYPFVPFLPAKAKTDIQFEGHPIEKDTMIALDIYGTMHREDVFENPDDFYPERFKDWDGSPFDLIPQGGGDYYTNHRCAGEWMTIIIMEETMKYFAERVTYDVPEQDLTVDLNSIPGYVKSGFIIENLREVVDRK

SEQ ID NO: 93 - protein, 423 aa, Salinicoccus carnicancri
MGTISRDKGLDNTLKVMKQGYLYMTNQRKRLGSENIFETRALGGKKIIVISGKDAAELFYDNDKIERSGTLPKRVVNTLFGKGAIHTTTGKKHIDRKALFMSLMTEGNLKHLRELTRNHWYMNTQKMEQMEQINIYRESIVQLTKIGTKWAGVQAPEEKIEEIATDMDVMIDSFKGLGGKFKGYKASVDARKRVEDWLEDQIIQTRKGKIFPPEGTALYEFAHWEDYKGNPMDSRLCAIDLMNTFRPLIAINRFVSFGLLAMHEHPITREKIKSDDDYAYMFSQEVRRFYPFVPFLPAKAKTDIQFEGHPIEKDTMIVLDIYGTMHREDVFENPDDFYPERFRDWDGSPFDLIPQGGGDYYTNHRCAGEWMTIIIMEETMKYFAGRVTYDVPEQDLTVDLNSIPGYVKSGFIIENLREVVDRK

SEQ ID NO: 94 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTTEEAEQNAKDMDIMIDSFKGLGQTIGGGYRKAKKARARVEQFLERQIIAVRKGKINAEPSTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIKNVRPRVQR

SEQ ID NO: 95 - protein, 425 aa, Staphylococcus agnetis
MAKQLPKDPGLDNTFKVLKEAYTYVPCRLEKFNSKAFQTTGMGMKPIAVINGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTAEEAEQNAQDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 96 - protein, 425 aa, Staphylococcus agnetis
MAKQLPKDPGLDNTFKVLKEAYTYVPCRLEKFNSKAFQTTGMGMKPIAVINGKEAAELFYNNDVMQREKTLPKRVVNTLFGKGAIHTTKGKVHVDRKALFMSLMTEENLKYLRELTRNHWFMHTEHMQNQKEVNIYKESIYVLTKIGFRWAGIHQTAEEAEQNAQDMDIMIDSFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLFVKLDQFPGKVTSGTIIQNVRPRVQR

SEQ ID NO: 97 - protein, 425 aa, Staphylococcus agnetis
MAKQLPKDPGLDNTFKVLKEAYTYVPCRLEKFNSKAFQTTGMGMKPIAVISGKEAAELFYNNDVMQREKTLPKRVVNTLFGKGAIHTTKGKVHVDRKALFMSLMTEENLKYLRELTRNHWFMHTEHMQNQREVNIYKESIYVLTKIGFRWAGIHQTAEEAEQNAKDMDIMIASFKGLGQTIGGGYRKAKKARARVEQFLEKQIIAVRKGKINAEPGTALYEFAHWEDYKGNPMDARLCAIDLMNVVRPLAAVNRFVSYAVKAMIEYDQERIKLQVSDDPNYAYKFAQEVRRIFPFVPFLPGKLKKNIEFDGYRIKKGTFTLLDVFGTTHDPELFENPYQFNPDRFENWDGSPFDLIPQGGGDFYTNHRCAGEWMTIIVMEETIKYFANKIDFEAPSQDLSVKLDQFPGKVTSGTIIQNVRPRVQR

SEQ ID NO: 98 - protein, 424 aa, Staphylococcus sp.
MAKKLPKDSGLDNTFKVLKEAYTYVPCRLEKFNTKAFQTKAMGMKPIVVISGKEAAELFYNNDVMQREKTLPKRVVNTLFGKGAIHTTKGKVHVDRKALFMSLMTEENLKYLRELARNHWFVHTEHMQTQDEVNIYKESIYVLTKIGFRWAGIRQTAEEAERNAKDMDVMIDSFKGLGQTLGSGYRKAKKARARVEQFLEDQIVAVRNGEIKAEPGTALYEFAHWEDYKGNQMDARLCAVDLMNVVRPLAAVNRFVSYAVKAMIEYDQERLKLQVTDDPNYAYKFAQEVRRIFPFVPFLPGKFKKTVEFDGYRIKKGTFTLLDVFGTTHDPDLFENPYQFNPDRFDNWDGSPFDLIPQGGGDFYTNHRCAGEWMTIIVMEETIKYFANKIDFDAPAQDLSVKLDQFPGKVTSGTLIKNVRPRLQ

SEQ ID NO: 99 - protein, 424 aa, Staphylococcus hyicus
MAKKLPKDSGLDNTFKVLKEAYTYVPCRLEKFNTKAFQTKAMGMKPIVVISGKEAAELFYNNDVMQREKTLPKRVVNTLFGKGAIHTTKGKVHVDRKALFMSLMTEENLKYLRELARNHWFVHTEHMQTQDEVNIYKESIYVLTKIGFRWAGIRQTAEEAERNAKDMDVMIDSFKGLGQTLGSGYRKAKKARARVEQFLEDQIVAVRNGEIKAEPGTALYEFAHWEDYKGNQMDARLCAVDLMNVVRPLAAVNRFVSYAVKAMIEYDQECLKLQVTDDPNYAYKFAQEVRRIFPFVPFLPGKFKKTVEFDGYRIKKGTFTLLDVFGTTHDPDLFENPYQFNPDRFDNWDGSPFDLIPQGGGDFYTNHRCAGEWMTIIVMEETIKYFANKIDFDAPAQDLSVKLDQFPGKVTSGTLIKNVRPRLQ

SEQ ID NO: 100 - protein, 424 aa, Salinicoccus qingdaonensis
MATIKRDKGLDNSVKVMKQGYLYTTNQRERLGVTDGVFETRALGGKRIIVLSGKDGAELFYDNDKIERSGTLPKRVVNTLFGKGAIHTTTGKVHIDRKALFMSLMTEGNLKYLRELTRNLWFANTQRMESMDQVNVYRESIIVLTKVGTRWAGVQAPEKEIENIATDMDIMIDSFKAIGSAFKGYREAKAARRRVEDWLEDQIIQTRKGKIHPPKGTALYEFAHWKDYKGEPMDSRLCGIDLMNTFRPLIAINRFVAFGALAMYENPVAREKIKQDDDYAYMFAQEVRRFYPFVPYLPAKAKVDFQYKGYEIEKDTMLALDIYGTMHDPNVWEDPNEFYPERFKDWDGSPFDLIPQGGGDYHTNHRCAGEWMTVIIMEETMKYFASRINYDVPEQDLTVDLNSLPGYIKSGFVIENVQENVDRT

SEQ ID NO: 101 - protein, 424 aa, Salinicoccus alkaliphilus
MATIKKDKGIDNTAKIAKQGYLYTTNQRERLGVKDGVFETRGLGGKRIIILSGKDGAELFYDNDKVERSGTLPKRVVNTLFGKGAIHTTTGKVHVDRKALFMSLMTEGNLQYLRELTRNHWLMNTQRMENMDQVNVYRESIILLTKVGTRWAGVQAPEKEIENIATDMDIMIDSFKAIGSAFKGYRASKAARRRVEDWLEDQIIQTRKGKIHPPKGTALYEFAHWKDYKGEPMDSRLCGIDLMNTFRPLIAINRFIAFGALAMHENPVAREKIKQDDDYAYMFAQEVRRFYPFVPYLPGKVKEDFQYKGYDFEKDTMLAIDIYGTMHDPDVWENPNEFYPERFKDWDGSPFDLIPQGGGDYYTNHRCAGEWMTIIIMQETMKYFASRITYDVPEQDLTVDLNSLPGYVKSGFIIENVREVVDRD

SEQ ID NO: 102 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYVVKAMLEFDQERIKLQVADDPNYAYKFAQEVRRIFPFVPFLPGKLKKDVEFDGFKIKKGTFTLLDIFGTTHDPELFENPYQFNPDRFDNWDGSPFDLIPQGGGDFYTNHRCAGEWMTIIIMEETIKYFATKIDFDAPVQDLSVKLDQFPGKVTSGPVIHRVRPRVQR

SEQ ID NO: 103 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDSELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 104 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVKFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 105 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYVVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 106 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEDMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 107 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 108 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEDMQRQDEVNIYKTSIYILTKIGFRWAGLQQTSEEAKRNARDMDIMIDSFQGLGQTFGGGYRKAKKARARVEKFLENQIVAVREGKMKAQPGTALYEFVHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 109 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQEEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDLNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 110 - protein, 425 aa, Staphylococcus chromogenes
MAKKLPKDSGLDNTFKILNEAYTYVPCRLEKFGTKAFETAALGMKPMTVISGKEAAELFYNNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAVNRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 111 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNCWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 112 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLMTEENLKYLRDLTRNYWFMHTEEMQRQDEVNIYKTSIYILTKIGFRWAGLQQTSEEAKRNARDMDIMIDSFQGLGQTFGGGYRKAKKARARVEKFLENQIVAVREGKMKAQPGTALYEFAHWKDYKGHQVDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 113 - protein, 425 aa, Staphylococcus chromogenes
MAKKLPKDSGLDNTFKILNEAYTYVPCRLEKFGTKAFETAAIGMKPMTVISGKEAAELFYDNELMQREKTLPKRVVNTLFGKGAIHTTKGKVHVDRKALFMSLITEENLKYLRDLTRNYWFMHTEDMQRQDEVNIYKTSIYILTKIGFRWAGLQQTSEEAKRNARDMDIMIDSFQGLGQTFGGGYRKAKKARARVEKFLENQIVAVREGKMKAQPGTALYEFVHWKDYKGHQMDPHLCAVELMNVVRPLAAINRFVSYAVKAMLEFDQERIKLQVADDPNYAYKFAQEVRRIFPFVPFLPGKLKKDVEFDGFKIKKGTFTLLDIFGTTHDPELFENPYQFNPDRFENWDGSPFDLIPQGGGDFYTNHRCAGEWMTIIIMEETIKYFATKIDFDAPVQDLSVKLDQFPGKVTSGPVIHRVRPRVQR

SEQ ID NO: 114 - protein, 422 aa, Nosocomiicoccus sp.
MAKIKRDKGLDNTLKVFKQGYLYTTNQRERLGAEVFETRALGGKSYVVLSGKEGAELFYDNDKIEREGGLPKRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYELAHWRDYEGNQMDSRLAGIDLMNTFRPLIAINRFVSYGVLALYEHPEAIRKMSTVEDYPYMFAQEVRRFYPFVPFLPGKTKVEVEHKGVTIPKDQRLVIDVYGTLHSEELWDQPNKFIPERFRDWDGSPFDMIPQGGGDYWTNHRCAGEWITIIIMEETMKYFANEITWDVPEQDLTIDLNSIPGYINSGMVINNVREKVDRF

SEQ ID NO: 115 - protein, 422 aa, Nosocomiicoccus sp.
MAKIKRDKGLDNTLKVFKQGYLYTTNQRERLGAEVFETRALGGKSYVVLSGKEGAELFYDNDKIEREGGLPKRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYELAHWRDYEGNQMDSRLAGIDLMNTFRPLIAINRFVSYGVLALYEHPEAIRKMSTVEDYPYMFAQEVRRFYPFVPFLPGKTKVEVEHKGVTIPKDQRLVIDVYGTLHSEELWDQPNKFIPERFKDWDGSPFDMIPQGGGDYWTNHRCAGEWITIIIMEETMKYFANEITWDVPEQDLTIDLNSIPGYINSGMVINNVREKVDRF

SEQ ID NO: 116 - protein, 423 aa, Nosocomiicoccus massiliensis
MMAKIKRDKGLDNTLRVFKQGYLYTTNQRERLGAEVFETRALGGKSYVVLSGKEGAELFYDNDKIEREGGLPRRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYEFAHWRDYEGNHMDSRLAGIDLMNTFRPLIAINRFVSYGVLALYENPEAIRKMSTVEDYPYMFAQEVRRFYPFVPFLPGKTKVEVEHKGVTIPKDQRLVIDVYGTLHSEELWDQPNKFIPERFKDWDGSPFDMIPQGGGDYWTNHRCAGEWITIIIMEETMKYFANEITWDVPEQDLTIDLNSIPGYINSGMVINNVREKVDRF

SEQ ID NO: 117 - protein, 422 aa, Nosocomiicoccus massiliensis
MAKIKRDKGLDNTLKVFKQGYLYTTNQRERLGAEVFETRALGGKSYVVLSGKEGAELFYDNDKIEREGGLPKRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYELAHWRDYEGNQMDSRLAGIDLMNTFRPLIAINRFVSYGVLALYEHPEAIRKMSTVEDYPYMFAQEVRRFYPFVPFLPGKTKVEVEHKGVTIPKDQRLVIDVYGTLHSEELWNQPNKFIPERFKDWDGSPFDMIPQGGGDYWTNHRCAGEWITIIIMEETMKYFANEITWDVPEQDLTIDLNSIPGYINSGMVINNVREKVDRF

SEQ ID NO: 118 - protein, 422 aa, Nosocomiicoccus massiliensis
MAKIKRDKGLDNTLRVFKQGYLYTTNQRERLGAEVFETRALGGKSYVVLSGKEGAELFYDNDKIEREGGLPRRVVNTLFGKGAIHTTTGKQHIDRKALFMSLMTEGNLEYVRKLTRNYWKANTARMEAMGDVNVYHESIVLLTRIGMRWAGVTAPEEEIERIAEDMDIMIDSFKGLGNAFKGYKSSKDARKRVEDWLEDQIIKTRNGEINPPKGSSLYEFAHWRDYEGNHMDSRLAGIDLMNTFRPLIAINRFVSYGVLALYENPEAIRKMSTVEDYPYMFAQEVRRFYPFVPFLPGKTKVEVEHKGVTIPKDQRLVIDVYGTLHSEELWDQPNKFIPERFKDWDGSPFDMIPQGGGDYWTNHRCAGEWITIIIMEETMKYFANEITWDVPEQDLTIDLNSIPGYINSGMVINNVREKVDRF

SEQ ID NO: 119 - protein, 422 aa, Nosocomiicoccus sp.
MAKIKRDKGLDNTLKVFKQGYLYTTNQRERLGAEVFETRALGGKSYVVLSGKEGAELFYDNDKIEREGGLPKRVVNTLFGKGAIHTTTGKQHIDRKALFMSLMTEGNLEYVRKLTRNYWKANTARMEAMGDVNVYQESIVLLTRIGMRWAGVTAPEEEIERIAEDMDIMIDSFKGLGNAFKGYKSSKDARKRVEDWLEDQIIKTRNGEINPPKGSSLYELAHWRDYEGNQMDSRLAGIDLMNTFRPLIAINRFVSYGVLALYEHREAIRKMSTVEDYPYMFAQEVRRFYPFVPFLPGKTKVEVEHKGVTIPKDQRLVIDVYGTLHSEELWDQPNKFIPERFRDWDGSPFDMIPQGGGDYWTNHRCAGEWITIIIMEETMKYFANEITWDVPEQDLTIDLNSIPGYINSGMVINNVREKVDRF

SEQ ID NO: 120 - protein, 347 aa, Salinicoccus sediminis
MGTIKRDKGLDNTLKVMKQGYLYITNQRKRLGSENIFETRALGGKKIIVISGKDAAELFYDNDKIERSGTLPKRVVNTLFGKGAIHTTTGKKHIDRKALFMSLMTEGNLKHLRELTRNHWYMNTQKMEQMDQINIYRESIVQLTKIGTKWAGVQAPEEKIEEIATDMDVMIDSFKGLGGKFKGYKASVDARKRVEDWLEDQIIQTRKGKIFPPEGTALYEFAHWEDYKGNPMDSRLCAIDLMNTFRPLIAINRFVSFGLLAMHEHPITREKIKSDDDYAYMFSQEVRRFYPFVPFLPAKAKTDIQFEGHPIEKDTMIALDIYGTMHREDVFENPDDFYPERFKDWDG

SEQ ID NO: 121 - protein, 414 aa, Pontibacillus halophilus
MNVNVAKDRLDQTISLVREGYAFIPNRLHSQHLDVYETKLLGQRIALIGGEEGANLFYDSSRMKRNGALPKPVLKTLFGEGGVHTKDGAAHAHRKQLFMSLMTPDALRRLSNLTTAYWEEYAMKWEKQRKVVLYDESRELLCQVACDWAGIPLGKKEVEQRTEDLSKMIDGFSALGPKHIESRRARNRSEKWIRSIIEDVRANKLETREGSAIHEMAFHLDENGDRLDTEIAAVELLNVIRPLVAISKFLAFGAKAYADYPETREKTKESDEYILRFAQEVRRYYPFAPFLGAKVKHDFTWQGYTFRENQLVMIDLFGTNHDPRLWENPDQFDPDRFKDWKGGLFDLIPQGGGDYYDGHRCPGEWPTIEVLKASFGYLSRKIDYKVPKQDHSYSLSKMPALPKSGMILKKVRRK

SEQ ID NO: 122 - protein, 420 aa, Macrococcus sp.
MSKLKESGFDHTLTMLKEGYTYIPNRTEKYQSPIFFNRVLGGKPCAIISGKEAAELFYNNDLIERKGTMPKRIVKTLFGKGAIHTTTGKVHIDRKALFMSLMTDENLSLLRQITKDAWENEVDSLIKVNEVNVYKKSSEVLTKVGLKWAGIEDTPKNIDKIAKEMDLMIDSFGQLGGAYKGYRAAQKARANVEAYLQNQIKKVRKGKLKPRENSALYAFSHWKDMNNKPMDLALCAVDLMNVIRPLVAINRFVTYGVLAIHEFNGEKDKLKFGTDDYVYQFVQEVRRFYPFVPYLPGKAKKKLTFDNQKIKKDQMILLDVYGTLHNEKLWEYPNRFDPTRFENWDGSPFDLIPQGGGDYHTNHRCAGEWMTIIIMEETMKFFTRTISFEVPPQDFTVDLTKLPGKVASGMNIRQVQALNN

SEQ ID NO: 123 - protein, 426 aa, Staphylococcus lentus
MGKNIPRERGLDNTITVLKEAYEYVPNRLEKHNTNIFELRALGGRRTVVISGKEAAELFYNNDLIERQGTLPKRIVNTLFGKGAIHTTGGKKHIDRKALFMSLMTEENLEYLRELTRNHWFMNTERMERMDEVNVYKESILMLTKIGFRWAGIIASPEEIEQCAADMDTMIDSFKNIGTVFKGYREAKQARDRVETFLENQIIAVREGKINPPQGTALYEFSHWEDFEGNLMDSRLCSIDLMNVVRPLVAINRFVSFGVKAFHDYPGEAERVFNNEDDYAYKFIQEVRRFYPFVPYLPGKAAVDIDFEGYTIEKDTFLVLDIYGTLHRDGLWENPERFYPNRFNDWDGSPFDLIPQGGGDYYTNHRCAGEWMTIIIMEETMKYFAQNISYDVKKDQDTSVNLNKLPGRVVDGMIITNVNALVNRNV

SEQ ID NO: 124 - protein, 426 aa, Staphylococcus lentus
MGKNIPRERGLDNTITVLKEAYEYVPNRLEKHNTNIFELRALGGRRTVVISGKEAAELFYNNDLIERQGTLPKRIVNTLFGKGAIHTTGGKKHIDRKALFMSLMTEENLEYLRELTRNHWFMNTERMERMDEVNVYKESILMLTKIGFRWAGIIASPEEIEQCAADMDTMIDSFKNIGTVFKGYREAKQARDRVETFLENQIIAVREGKINPPQGTALYEFSHWEDFEGNLMDSRLCSIDLMNVVRPLVAINRFVSFGVKAFHDYPGEAERVFNNEDDYAYKFIQEVRRFYPFVPYLPGKAAVDIDFEGYTIEKDTFLVLDIYGTLHREGLWENPERFYPNRFNDWDGSPFDLIPQGGGDYYTNHRCAGEWMTIIIMEETMKYFAQNISYDVKKDQDTSVNLNKLPGRVVDGMIITNVNALVNRNV

SEQ ID NO: 125 - protein, 426 aa, Staphylococcus lentus
MGKNIPRERGLDNTITVLKEAYEYVPNRLEKHDTNIFELRALGGRRTVVISGKEAAELFYNNDLIERQGTLPKRIVNTLFGKGAIHTTGGKKHIDRKALFMSLMTEENLEYLRELTRNHWFMNTERMERMDEVNVYKESILMLTKIGFRWAGIIASPEEIEQCAADMDTMIDSFKNIGTVFKGYREAKQARDRVETFLENQIIAVREGKINPPQGTALYEFSHWEDFEGNLMDSRLCSIDLMNVVRPLVAINRFVSFGVKAFHDYPGEAERVFNNEDDYAYKFIQEVRRFYPFVPYLPGKAAVDIDFEGYTIEKDTFLVLDIYGTLHREGLWENPERFYPNRFNDWDGSPFDLIPQGGGDYYTNHRCAGEWMTIIIMEETMKYFAQNISYDVKKDQDTSVNLNKLPGRVVDGMIITNVNALVNRNV

SEQ ID NO: 126 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEENLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 127 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEENLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 128 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQVAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 129 - protein, 426 aa, Macrococcus brunensis
MAKRMPKVKGLDQTLTVLKEGYEFIPNRTKELNTNVFEMRVLGGKKAVVISGKEAAELFYDNDKTERSGTLPKRLVKTLFGKGAIHTTTGDKHKNRKTLFMSLMTDNNLEYLRKLTRNYWFEDTARLQAMDQTNVYLESTLVLLKVGFRFADVPFDTPERMEQYAKDMNAMVDSFGSIGTAYSGYKRALNARDRVEDYLEKQIIATRKGKLFPEEGSGLYEFSHWKDMNGKQMDSRLAAVDLMNTIRPLVAINRFISFGVLAMHEYPGERARVAQNDNSYAYKFAQEVRRYYPFVPFLPGKAKKNITFDGYKIEKDTMLILDIFGTLHSESLWQNAEQFYPERFENWDGSPFDLIPQGGGDYWTNHRCAGEWMTVIIMEESMKYFAQEIQYDVPAQDFTVDRTKLPGQINSGMIIENIRNNFDRRR

SEQ ID NO: 130 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALTEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 131 - protein, 422 aa, Staphylococcus pseudintermedius
MAKKLPKDTGLDNTLKMIYEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 132 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 133 - protein, 422 aa, Staphylococcus sp.
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 134 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 135 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 136 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFSQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 137 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFSQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 138 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDILGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 139 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 140 - protein, 422 aa, Staphylococcus pseudintermedius
MAKKLPKDTGLDNTLKIINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPAQDLSVKLSQFPGKVTSGTMIKNVYPRI

SEQ ID NO: 141 - protein, 422 aa, Staphylococcus pseudintermedius
MAKKLPKDTGLDNTLKMINEAYTYVPKRLEKFGTKAFETRALGMKTIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPAQDLSVKLSQFPGKVTSGTMIKNVYPRI

SEQ ID NO: 142 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQETGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 143 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQLNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 144 - protein, 422 aa, Staphylococcus pseudintermedius
MANKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 145 - protein, 422 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLCELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPFDLIPQGGGDFYTNHRCAGEWMTVIVMEETIQYFANKIDFVVPAQDLSVKLSQFPGKVTSGTMIKNVYPRI

SEQ ID NO: 146 - protein, 371 aa, Staphylococcus pseudintermedius
GKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLRQTDEQAVQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDILGTTHDPELFENPYQFNPDRFDNWDGSPFDLIPQGGGDFYTNHRCAGEWMTVIVMEETIQYFANKIDFVAPPQDLSVKLSQFPGKVTSGTMIKNVYPRI

SEQ ID NO: 147 - protein, 352 aa, Staphylococcus pseudintermedius
MAKKLPKDTGLDNTLKMINEAYTYVPKRLEKFGTKAFETRALGMKPIVVISGKAAAELFYDNDKISRKGTLPKRIVHTLFGKGAIHTTEGKVHVDRKALFMSLMTEKNLKYLRELTRNYWFMHTERMQNKDEVNVYQEAGLILTKVGFRWAGLKQTDEQAAQNAEDMNTMIDSFSGLGQSLKGYREAKKARARVEQFLQEQIEAVRVGQQYAEPGTALYEFAHWKDLNDQPMDPHLCAVDLMNIVRPLVAVNRFVSYGVKALIEFDQERKKLQVTNDPNYAYKFAQEVRRIFPFVPFLPGRLKKTVEFDGFKLKKGTLTVLDIFGTTHDPELFENPYQFNPDRFDNWDGSPF

SEQ ID NO: 148 - protein, 423 aa, Jeotgalicoccus saudimassiliensis
MMASLKRDKGLDNTLKLMKQGYLYTTNQRNRLDTTVFETKVLGGKPFAVVTGKEGAEMFYNNDVVQREGMLPKRIVNTLFGKGAIHTIDGKKHIDRKALFMSLMTEGNLDYVRELTRTLWRANTQRMESMDQVNIYRESIVLLTKVGTRWAGVQAPPEDIERIATDMDIMIDSFKGLGGAFKGYKESKAARRRVEDWLEEQILETRKGNIHPPEGTALYEFAHWEDYLGNPMDSRNCAIDLMNTFRPLIAINRFVSFGLLAMHDNPITREKIKSEPDYAYKFAQEVRRFYPFVPFLPGKAKVDIDFQGVTIPAGHGLALDVYGTMHDETLWEDPNEFRPERFEDWDGSPFDLIPQGGGDYWTNHRCAGEWITVIIMEETMKYFAEKVTYDVPEQDLTVDLNSIPGYVKSGFIISNVQEVVDRK

SEQ ID NO: 149 - protein, 423 aa, Jeotgalicoccus sp.
MMASLKRDKGLDNTLKLMKQGYLYTTNQRNRLNTTVFETKVLGGKPFAVVTGKEGAEMFYNNDIVQREGMLPKRIVNTLFGKGAIHTIDGKKHIDRKALFMSLMTEGNLNYVRELTRTLWQANTQRMESMDQVNIYRESIVLLTKVGTRWAGVQAPPEDIERIATDMDIMIDSFKGLGGVFKGYKESKAARRRVEDWLEEQILETRKGNIHPPEGTALYEFAHWEDYLGNPMDSRNCAIDLMNTFRPLIAINRFVSFGLLAMHDNPITREKIKSEPDYAYKFAQEVRRFYPFVPFLPGKAKVDIDFQGVTIPAGHGLAIDVYGTMHDETLWENPNEFRPERFEDWDGSPFDLIPQGGGDYWTNHRCAGEWITVIIMEETMKYFAEKVTYDVPEQDLTVDLNSIPGYVKSGFIISNVQEVVDRK

SEQ ID NO: 150 - protein, 422 aa, Jeotgalicoccus saudimassiliensis
MASLKRDKGLDNTLKLMKQGYLYTTNQRNRLDTTVFETKVLGGKPFAVVTGKEGAEMFYNNDVVQREGMLPKRIVNTLFGKGAIHTIDGKKHIDRKALFMSLMTEGNLDYVRELTRTLWRANTQRMESMDQVNIYRESIVLLTKVGTRWAGVQAPPEDIERIATDMDIMIDSFKGLGGAFKGYKESKAARRRVEDWLEEQILETRKGNIHPPEGTALYEFAHWEDYLGNPMDSRNCAIDLMNTFRPLIAINRFVSFGLLAMHDNPITREKIKSEPDYAYKFAQEVRRFYPFVPFLPGKAKVDIDFQGVTIPAGHGLALDVYGTMHDETLWEDPNEFRPERFEDWDGSPFDLIPQGGGDYWTNHRCAGEWITVIIMEETMKYFAEKVTYDVPEQDLTVDLNSIPGYVKSGFIISNVQEVVDRK

SEQ ID NO: 151 - protein, 441 aa, Macrococcus caseolyticus
MRVEFTINYINVEGISMSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 152 - DNA, 1266 bp, Artificial Sequence
  Codon optimized artificial DNA sequence
atggcgaccctgaagcgtgacaaaggcctggataacaccctgaaggtgctgaaacagggttacctgtataccaccaaccaacgtaaccgtctgaacaccagcgttttccagaccaaagcgctgggtggcaagccgtttgtggttgtgaccggcaaagagggtgcggaaatgttctacaacaacgacgttgtgcaacgtgagggcatgctgccgaaacgtatcgtgaacaccctgttcggcaagggtgcgattcacaccgttgacggcaagaaacacgtggatcgtaaggcgctgtttatgagcctgatgaccgagggtaacctgaactacgttcgtgaactgacccgtaccctgtggcacgcgaacacccagcgtatggagagcatggacgaagtgaacatctatcgtgaaagcattgttctgctgaccaaagtgggcacccgttgggcgggtgttcaagctccgccggaggatatcgaacgtattgcgaccgacatggatatcatgattgacagcttccgtgcgctgggtggcgcgtttaagggttataaagcgagcaaagaggcgcgtcgtcgtgtggaagattggctggaggaacagatcattgagacccgtaagggcaacatccacccgccggaaggtaccgcgctgtacgagttcgcgcactgggaagactatctgggcaacccgatggatagccgtacctgcgcgattgacctgatgaacacctttcgtccgctgatcgcgattaaccgtttcgtgagctttggtctgcacgcgatgaacgagaacccgatcacccgtgagaagattaaaagcgaaccggactacgcgtataaattcgcgcaggaagtgcgtcgttactatccgttcgttccgtttctgccgggcaaggcgaaagttgacatcgattttcaaggtgttaccatcccggcgggcgtgggtctggcgctggatgtttacggtaccacccacgacgagagcctgtgggacgatccgaacgaattccgtccggagcgttttgaaacctgggacggcagcccgttcgatctgatcccgcagggtggcggtgattattggaccaaccaccgttgcgcgggcgagtggatcaccgtgatcattatggaggaaaccatgaaatacttcgcggaaaagattacctatgacgtgccggagcaagacctggaagttgatctgaacagcatcccgggctacgttaaaagcggttttgttattaagaacgtgcgtgaggttgtggatcgtacc

SEQ ID NO: 153 - protein, 436 aa, Jeotgalicoccus sp.
MHHHHHHENLYFQGMATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 154 - DNA, 1308 bp, Artificial Sequence
  Codon optimized artificial DNA sequence
catatgcaccaccaccaccaccactcaaaacgcgttcccaaagatagagggattgataacagcctgaagatcatgaaagagggttacgaatatgttccggcgcgtatgaagaagttcaacaccaacatcttcgagacccgtgtgctgggtggcaagaccgcggtggttatcagcggcaaagatgcggcggagctgttctacgacaacgataagaccgaacgtaaaggcaccctgccgaagcgtgtggttaaaaccctgttcggcaagggtgcgattcacaccaccaccggtaagaaacacatcgatcgtaaagcgctgtttatgagcctgatgaccgacgagaacctggcgtacctgcgtaagctgacccgtatttattggtttcagaacatcgagcacatgcagtacaagcaaaaagtgaacgtttatgaggaagcgaccgaactgctgaccaaagttggcctgcgttgggcgggtattgtggatcacccggaaaacatccaaaagatggcggacgatatgaacaaaatgattgacagcttcagcgcgatcggcagcctgtacggtggctatcgtgaggcgaagaaagcgcgtgcgcgtgttgagcagtttctggaagaccaaattaccgcggtgcgtaagggcaaaatccacccggaaaagggcaccgcgctgtacgagttcagccactgggaagacatgaacggtaaaccgatggatgcgcgtctgtgcgcggtggacctgatgaacgttattcgtccgctggttgcgatcaacaagttcgtgagctttggcgttctggcgctgcacgagttcccgggtgaacgtgtgcgtgttgcgctgaacgagggcgattacgcgtataaatttgtgcaggaagttcgtcgttactatccgttcgtgccgtttctgccgggcaaggcgaaagagaacattaccttcgacggttacaagatccaaaaagacaccatgatgctgctggatatttatggcaccctgcaccgtgacgacctgttcagcgagccggaacgtttcaacccgtaccgttttgacaactgggatggcagcccgtttgatctgatcccgcagggtggcggtgactactataccaaccaccgttgcgcgggcgagtggatgaccatcattatcatggaggaaaccatgaagttctttgcgaacgaaatcagctatgacgttccgccgcaagacttcaccgtggataccaccaagtttccgggcaaagttgcgagcggtatggatattgaaaacatccgtgtgaacatcgaccgtaccaaataatgaaagctt

SEQ ID NO: 155 - protein, 431 aa, Micrococcus lylae
MHHHHHHSKRVPKDRGIDNSLKIMKEGYEYVPARMKKFNTNIFETRVLGGKTAVVISGKDAAELFYDNDKTERKGTLPKRVVKTLFGKGAIHTTTGKKHIDRKALFMSLMTDENLAYLRKLTRIYWFQNIEHMQYKQKVNVYEEATELLTKVGLRWAGIVDHPENIQKMADDMNKMIDSFSAIGSLYGGYREAKKARARVEQFLEDQITAVRKGKIHPEKGTALYEFSHWEDMNGKPMDARLCAVDLMNVIRPLVAINKFVSFGVLALHEFPGERVRVALNEGDYAYKFVQEVRRYYPFVPFLPGKAKENITFDGYKIQKDTMMLLDIYGTLHRDDLFSEPERFNPYRFDNWDGSPFDLIPQGGGDYYTNHRCAGEWMTIIIMEETMKFFANEISYDVPPQDFTVDTTKFPGKVASGMDIENIRVNIDRTK

SEQ ID NO: 156 - protein, 453 aa, Staphylococcus massiliensis
MFVDSILVLRLNLLKTGIQLEMKNGGIKVAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 157 - protein, 425 aa, Staphylococcus massiliensis
MAKKLPKVKGLDNTVDIIKGGYTYVPGKLEEFDSKAFEVRALGGKKIAVMSGKEAAEIFYDNEKMERQGTLPKRIVNTLFGKGAIHTTAGKKHVDRKALFMSLMTDENLNYLRELTRNYWFMNTERMQSMDKVNVYNESIYMLTKIGFRWAGIIQTPEEAEQNAKDMDTMINSFVSLGSAYKGYKKAKKARKRVEDFLEKQIIDVRKGKLHPEEGTALYEFAHWEDLNDNPMDSHLCAVDLMNVVRPLAAINRFISYGVKVLIEFDQEKEKLRLENNEDYAYKFAQEVRRIFPFVPYLPGRAAVDLEYDGYKIPAGMMTALDVYGTTHDEDLWENPDQFNPNRFDNWDGSPFDLIPQGGGDFYTNHRCAGEWITVIIMEETMKYFANKIEFDVPSQDLSVKLDKLPGNVTSGTIISNVRPRVARK

SEQ ID NO: 158 - protein, 422 aa, Jeotgalicoccus halophilus
MASLKRDKGLDNTLKVMKQGYLYTTNQRNRLNTTVFETKALGGKPFAVVTGKEGAEMFYNNDIVQREGMLPKRIVNTLFGKGAIHTIDGKKHVDRKALFMSLMTEGNLNYVRELTRTLWQANTQRMESMDEVNIYRESIVLLTKVGTRWAGVQAPPEQIERIATDMDIMIDSFKGLGGVFKGYKESKAARRRVEDWLEDQILETRKGNIHPPEGTALYEFAHWEDYLGNPMDSRNCAIDLMNTFRPLIAINRFVSFGLLAMHDNPVSREKIKSEPDYAYKFAQEVRRYYPFVPFLPGKAKTDIDFQGVTIPAGHGLAIDVYGTLHDESLWEDPNEFRPERFEGWDGSPFDLIPQGGGDYWTNHRCAGEWITVIIMEETMKYFAEKVTYDVPEQDLTVDLNSIPGYVKSGFVIKNVQEVVDRR

SEQ ID NO: 159 - DNA, 1332 bp, Artificial Sequence
  Codon optimized artificial DNA sequence
catatgcatcaccaccaccaccacgagaacctgtacttccagggcatggcgaagcgtatgccgaaggtgaaaggtctggaccaaaccctgaccgttctgaaagagggttatgaattcattccgaaccgtacccgtaaactgaacaccaacgtttttgagattcgtatcctgggtggcaagaaagcggtggttatcagcggcaaagaggcggcggaactgttttacgacaacgataaaaccgaacgtgcgggtaccctgccgaagcgtctggtgaaaaccctgttcggcaagggtgcgattcacaccaccaccggtgataagcacaaaaaccgtaaaagcctgtttatgagcctgatgaccgacaacaacctggagtacctgcgtaaactgacccgtaactattggttcgaagataccgcgcgtctgcaggcgatggaccaaaccaacgtgtaccaggaagcgaccctggttctgctgaagattggcttccgttttgcggacgttccgttcgataccccggagcgtatggaacaatatgcgaaagacatgaacgcgatggtggatagctttggcagcatcggtaccgcgtacaccggttatcgtcgtgcgctgaacgcgcgtgaccgtgttgaggattacctggaaaagcagatcattgcgacccgtaagggcaaactgttcccggaggaaggcagcggtctgtatgagtttagccactggaaggacatgaacggtaaacacatggatagccgtctggcggcggtggacctgatgaacaccatccgtccgctggttgcgattaaccgtttcatcagctttggcgtgctggcgatgcacgagtacccgggtgaacgtgcgcgtgtggcgcagaacgacaacagctacgcgtataaattcgcgcaagaagtgcgtcgttactatccgttcgttccgtttctgccgggcaaggcgaaacaaaacattaccttcgagggttacaagatcgacaaagataccatgctgattctggatatctttggtaccctgcacagcgaaagcctgtgggagaacgcggaacagttctatccggagcgttttgaaaactgggacggcagcccgtttgatctgattccgcaaggtggcggtgattactataccaaccaccgttgcgcgggcgagtggatgaccatcattatcatggaggaaaccatgaagtacttcgcgcgtgaaatccagtatgacgtgccgccgcaagactttaccgttgatcgtaccaaactgccgggccagattaacagcggtatgattatcgagaacatccgtaacaacttcgatcgtcgtcgttaactcgag

SEQ ID NO: 160 - DNA, 1320 bp, Artificial Sequence
  Codon optimized artificial DNA sequence
catatgcatcaccaccaccaccacgagaacctgtacttccagggcatggcgaagaaactgccgaaagataccggtctggacaacaccctgaagatcattaacgaagcgtacacctatgtgccgaagcgtctggagaaattcggcaccaaggcgtttgaaacccgtgcgctgggcatgaaaccggtggttgtgatcagcggcaaggcggcggcggagctgttttatgacaacgataaaatcagccgtaagggcaccctgccgaaacgtattgttcacaccctgttcggcaagggtgcgatccacaccaccgagggtaaagttcacgtggatcgtaaggcgctgtttatgagcctgatgaccgagaagaacctgaaatacctgcgtgaactgacccgtaactattggttcatgcacaccgagcgtatgcagaacaaagatgaggttaacgtgtaccaagaagcgggcctgattctgaccaaagtgggctttcgttgggcgggtctgaagcagaccgacgagcaggcggcgcaaaacgcggaagatatgaacaccatgatcgacagcttcagcggcctgggtcagagcctgaaaggttatcgtgaagcgaagaaagcgcgtgcgcgtgtggagcaatttctgcaggaccaaatcgaagcggtgcgtgcgggtcagcaatatgcggagccgggtaccgcgctgtatgaattcgcgcactggaaggacctgaacgatcaaccgatggatccgcacctgtgcgcggttgacctgatgaacattgtgcgtccgctggttgcggtgaaccgtttcgttagctacggtgtgaaagcgctgatcgagtttgatcaggagcgtaagaaactgcaagttacccacgacccgaactacgcgtataagttcgcgcaggaagtgcgtcgtattttcccgtttgtgccgtttctgccgggccgtctgaaacaaaccgtggaattcgatggctttaagctgaagaaaggtaccctgaccgttctggatatcttcggtaccacccacgacccggagctgtttgaaaacccgtatcagttcaacccggaccgttttgataactgggacggcagcccgttcgatctgattccgcaaggtggcggtgacttttacaccaaccaccgttgcgcgggcgagtggatgaccgttatcgtgatggaggaaaccattcagtatttcgcgaacaaaatcgactttgaagcgccggcgcaggatctgagcgttaaactggaccaattcccgggcaaggtgaccagcggtaccatcattaagagcgtttacccgcgtatttaactcgag

SEQ ID NO: 161 - DNA, 1317 bp, Artificial Sequence
  Codon optimized artificial DNA sequence
catatgcatcaccaccaccaccacgagaacctgtacttccagggcatgaacaagattccgaaggacaaaggtctggataacaccctgaagatcgtgaaagaagcgtacacctatgttccgcgtcgtctggaggcgttcgacaccaacgtgtttgaaacccgtgcgctgggcatgaagaaagttctggtggttagcggtaagaaagcggcggagctgttttatgataacgaaaagatcagccgtaaaggcgcgctgccgaaacgtattgtgaacaccctgtttggtcagggtgcgatccacaccaccgagggcaagcaacacgttgaccgtaaagcgctgtttatgagcctgatgaccaccaagaacctgaaatacctgcgtgaactgacccgtaactattggttcgcgcacacccagcgtatggaggacatggatgaagtgaacgtttacgaggaagcgaccctgctgctgaccaagattggctttcgttgggcgggtctgaaacagacccacgaggaagcggtgcaaaacgcgaaggacatggatctgctgatcgatagctttagcgcgctgggtcaaagcccggcgggttataagaaagcgaagaaagcgcgtgagcgtgtggaaaagtttctggagcaccagattaaagcggttcgtgagggcgaacaatacgcggcgccgaacaccgcgctgtatgaattcgcgcactggaaggacctgaacgataaaccgatggacctgcacctgtgcgcggtggatctgatgaacgtggtgcgtccgctggttgcggttaaccgtttcgtgagctacgcggttaaagcgctgatcgagtttgaccaggaacgtctgaagctgcaggttaccaaagatcaaaactacgcgtataagtttgcgcaggaagtgcgtcgtattttcccgtttgttccgttcctgccgggccgtctgaaacaaaacatcgaatttgacggttacagcctgaagaaaaacaccctgatcattctggacgtgttcggtaccacccacgatccgaagctgtttgagaacccgtatcagttcaacccgaaccgttttgaagactgggatggcagcccgttcgacctgatcccgcaaggtggcggtgatttttacaccaaccaccgttgcgcgggcgagtggatgaccgtgatcgttatggaggaaaccatcaactacttcgcgaacaagatcgacttcagcgtgccgacccaggacctgagcgttaagctggatcaattcccgggcaaagttaccagcggtaccgtgatccgtaacgttcgtccgcgtcgttaactcgag

SEQ ID NO: 162 - DNA, 1326 bp, Artificial Sequence
  Codon optimized artificial DNA sequence
catatgcatcaccaccaccaccacgagaacctgtactttcagggcatgagcaagcaactgaacatcccgaaagaggaaggcctggaccacagcctggcgctgatccaggaaggttaccaatatattaccagccgtcgtaccaagtttaaaagcgacctgttcgaaacccgtctgctgggccagaaagtggtttgcattggtggccaagagggtgcggaactgttttatgacaacgagaagttcgaacgtaagggtgcggttccgaagcgtatccagaaaagcctgtttggcgaaaacgcgattcacaccctggatggcgaggcgcacgaacaccgtaagctgctgttcatgagcctgatgaccgaggaacgtctggagctgctggcggaactgacccgtgaggaatggcgtctggcggcggcggagtgggcgaaaatggaacaggtggttctgtttgacgaggttcagcaaattctgtgccgtgttgcgtgccgttgggcgggcgttccgatcgatgcgattgaagtgaagccgctggcgcgtgactttattgcgatggttgatgcgtttggtgcggtgggtccgcgtcaccaaaagggccgtaaagcgcgtaagaaaatggaggactacttcaagggtctgatcattgaaatccgtcaccgtcgtatcgagccgattgaaggtaccgcgctgcacaaaattgcgtggcaccgtgaccacaaggagaaacacctggatgaacacaccgcggcggtggagctgctgaacgttctgcgtccgatcgtggcgattacctacctggttacctttggcgcgctggcgctgttcgagcacaacgtgtggaaggaaaaactgcagagcggtgatgaggaaagcgttcagatgttcgcgcaagaagtgcgtcgtttctatccgtttgcgccgttcctgggtgcgaaggttaaaaacggctttgagtggaagggtcacaccttcgaaaaaggccaactggtgctgctggacgtttacggtaccaaccacgataaccgtctgtgggaggaaccgtatgtgtttaacccggagcgtttccgtaactggaacggtggcatgtttgacctgatcccgcagggtggcggtgatgagtacaccggccaccgttgcccgggtgaaatggcgaccatccaagtgatgaaaaccagcttcagctttctgaccaacgagatgatttataacgtgccggttggtcaggacctgagctacagcctgagccgtatgccgacctatccggaaagcggcttcatcattcagggtgttagcatcaagagccaaaccaccgtgtaactcgag

SEQ ID NO: 163 - DNA, 1326 bp, Artificial Sequence; identical to SEQ ID NO: 162
  Codon optimized artificial DNA sequence

SEQ ID NO: 164 - DNA, 1329 bp, Artificial Sequence
  Codon optimized artificial DNA sequence
catatgcatcaccaccaccaccacgagaacctgtacttccagggcatgggtaaaagcattccgcgtgaccgtggcatggataacaccgttaaggcgctgaagaaaggttacacctatattccggagctgcgtgaacgtctgaacagcaacgatatcgttgcgacccgtgcgctgggtggcaagaaaaccgtgatcattagcggcaaagacgcggcggagctgttttacgacaacgataagatcgaacgtcaaggtaccctgccgccgcgtgtggttaagaccctgttcggcaaaggtgcgattcacaccaccagcggcaagacccacatcgatcgtaaagcgctgtttatgagcctgatgaccgaggaaaacctgagctatctgcgtgagctgacccgtctggaatggtactataacaccacccgtatgagccacatggacgaggtgaacgtttacaaagaagcgatttatgtgctgaccaaggttggcatcaaatgggcgggtctgcacgcgagcgaggatgaaattaaccagtacgcggaggacatggataccatgatcgacagcttcaaagcgattggcaccatgtttaagggttataaagaagcgcgtaaggcgcgtgatcgtgttgaaacctggctggaagcgcagattcaaggcgtgcgtgatggtgaaattaccccgccggaaggtaccgcgctgtacgagttcgcgcactggaaagactatgaaggtaacccgatggatgcgcgtctggcggcgatcgacctgatgaacgtgattcgtccgctggttgcgatcaaccgtttcgttgcgtttggtaccctggcgatgtacgagttcccgaaggaacgtaccaaagtggcgcagaacgaggaagattacgtgtataagtttatccaagaggttcgtcgtttctacccgtttgtgccgtatctgccgggcaaggcgggtaaagacattgagttccgtggctacgaaatcaagaaagacacctttatggtgctggatgtttatggtaccctgcaccgtgaggacctgtgggaaaacgcggatcagttcattccggagcgttttgaagactgggatggcagcccgttcgatctgatcccgcaaggtggcggtgactactataccaaccaccgttgcgcgggcgagtggatgaccgtgattgttatggaggaaagcatgaaatactttgcgcaggcgatcaagtatgaagttccggcgcaagacctgaccgtggatctgaacaagctgccgggccgtgttaaaagcggtttcatcattaagaacgtggaacgtgacatcaaccgtttttaactcgag

SEQ ID NO: 165 - DNA, 1320 bp, Artificial Sequence
  Codon optimized artificial DNA sequence
catatgcatcaccaccaccaccacgaaaacctgtactttcagggcatggcgaagattaaacgtgacaagggtctggataacaccctgaaggttttcaaacagggctacctgtataccaccaaccaacgtgagcgtctgggtgcggaagtgtttgaaacccgtgcgctgggtggcaaaagctacgtggttctgagcggcaaggaaggtgctgagctgttctatgacaacgataaaatcgaacgtgagggtggcctgccgaagcgtgtggttaacaccctgttcggcaaaggtgcgattcacaccaccaccggcaaacaacacatcgatcgtaaggcgctgtttatgagcctgatgaccgaaggtaacctggagtacgttcgtaaactgacccgtaactattggaaggcgaacaccgcgcgtatggaagcgatgggcgacgtgaacgtttaccacgagagcattgttctgctgacccgtattggtatgcgttgggcgggtgtgaccgcgccggaggaagagattgaacgtatcgcggaggacatggatattatgatcgatagctttaagggcctgggtaacgcgttcaagggttataaaagcagcaaggacgcgcgtaaacgtgtggaagactggctggaggatcagatcattaagacccgtaacggcgaaattaacccgccgaaaggtagcagcctgtacgaactggcgcactggcgtgactatgagggcaaccaaatggatagccgtctggcgggtatcgacctgatgaacacctttcgtccgctgattgcgatcaaccgtttcgtgagctacggtgttctggcgctgtatgaacacccggaggcgatccgtaaaatgagcaccgttgaagactacccgtatatgttcgcgcaggaagtgcgtcgtttctacccgtttgtgccgttcctgccgggcaagaccaaagtggaagttgagcacaaaggtgtgaccattccgaaggaccagcgtctggtgatcgatgtttatggtaccctgcacagcgaagagctgtggaaccaaccgaacaaatttattccggaacgtttcaaggactgggatggcagcccgttcgatatgatcccgcagggtggcggtgactactggaccaaccaccgttgcgcgggcgagtggattaccatcatcatcatggaagaaaccatgaagtacttcgcgaacgaaattacctgggatgttccggagcaagacctgaccatcgatctgaacagcattccgggctacatcaacagcggtatggtgatcaacaacgttcgtgagaaagtggaccgtttttaactcgag

SEQ ID NO: 166 - DNA, 1296 bp, Artificial Sequence
  Codon optimized artificial DNA sequence
catatgcatcaccaccaccaccacgagaacctgtacttccagggcatgaacgtgaacgttgcgaaagaccgtctggatcaaaccatcagcctggttcgtgagggttacgcgtttattccgaaccgtctgcacagccagcacctggacgtgtatgaaaccaaactgctgggccaacgtatcgcgctgattggtggcgaggaaggtgcgaacctgttctatgatagcagccgtatgaagcgtaacggtgcgctgccgaaaccggttctgaagaccctgttcggcgagggtggcgtgcataccaaagatggtgcggcgcatgcgcaccgtaagcagctgtttatgagcctgatgaccccggatgcgctgcgtcgtctgagcaacctgaccaccgcgtactgggaggaatatgcgatgaagtgggaaaaacagcgtaaggtggttctgtacgacgagagccgtgaactgctgtgccaagtggcgtgcgattgggcgggtatcccgctgggtaagaaagaggtggaacaacgtaccgaggacctgagcaaaatgatcgatggcttcagcgcgctgggtccgaagcacattgaaagccgtcgtgcgcgtaaccgtagcgagaaatggattcgtagcatcattgaagacgttcgtgcgaacaagctggaaacccgtgagggcagcgcgatccacgagatggcgtttcacctggatgaaaacggtgaccgtctggataccgagattgcggcggtggaactgctgaacgttatccgtccgctggttgcgattagcaaattcctggcgtttggcgcgaaggcgtacgcggactatccggaaacccgtgagaagaccaaagagagcgatgaatacatcctgcgtttcgcgcaggaagttcgtcgttactatccgttcgcgccgtttctgggcgcgaaggtgaaacacgacttcacctggcagggttatacctttcgtgagaaccaactggttatgatcgacctgttcggtaccaaccacgatccgcgtctgtgggaaaacccggaccagttcgacccggatcgttttaaagactggaagggtggcctgtttgatctgattccgcaaggtggcggtgactactatgatggtcatcgttgcccgggcgagtggccgaccatcgaagttctgaaagcgagctttggctacctgagccgtaagattgactataaagtgccgaagcaagatcacagctacagcctgagcaaaatgccggcgctgccgaagagcggtatgatcctgaagaaagtgcgtcgtaagtaactcgag

SEQ ID NO: 167 - DNA, 1314 bp, Artificial Sequence
  Codon optimized artificial DNA sequence
catatgcatcaccaccaccaccacgagaacctgtactttcagggcatgagcaaactgaaggaaagcggtttcgatcacaccctgaccatgctgaaagagggctacacctatattccgaaccgtaccgaaaagtaccaaagcccgatcttctttaaccgtgttctgggtggcaaaccgtgcgcgatcattagcggcaaagaggcggcggaactgttttataacaacgacctgattgagcgtaaaggtaccatgccgaaacgtatcgttaagaccctgtttggcaagggtgcgattcacaccaccaccggtaaagtgcacatcgatcgtaaggcgctgttcatgagcctgatgaccgacgaaaacctgagcctgctgcgtcagattaccaaagacgcgtgggagaacgaagttgatagcctgatcaaggtgaacgaggtgaacgtttacaagaaaagcagcgaagtgctgaccaaagttggcctgaagtgggcgggtattgaggacaccccgaaaaacattgataaaatcgcgaaggaaatggacctgatgatcgatagcttcggccagctgggtggcgcgtacaaaggttatcgtgcggcgcaaaaggcgcgtgcgaacgttgaggcgtacctgcagaaccaaattaagaaagtgcgtaaaggcaagctgaaaccgcgtgaaaacagcgcgctgtatgcgtttagccactggaaagacatgaacaacaagccgatggatctggcgctgtgcgcggtggacctgatgaacgttattcgtccgctggttgcgatcaaccgtttcgtgacctacggtgttctggcgatccacgagttcaacggcgaaaaggataaactgaagtttggtaccgacgattacgtttatcagttcgtgcaagaggttcgtcgtttctacccgtttgtgccgtatctgccgggcaaagcgaagaaaaagctgacctttgacaaccagaagattaaaaaggaccaaatgatcctgctggatgtgtacggtaccctgcacaacgagaaactgtgggaatatccgaaccgtttcgatccgacccgttttgaaaactgggacggcagcccgttcgatctgatcccgcagggtggcggtgactatcacaccaaccaccgttgcgcgggcgagtggatgaccatcattatcatggaggaaaccatgaagttctttacccgtaccattagcttcgaagttccgccgcaagactttaccgtggatctgaccaaactgccgggcaaggttgcgagcggtatgaacatccgtcaggtgcaagcgctgaacaactaactcgag

SEQ ID NO: 168 - DNA, 1308 bp, Artificial Sequence
  Codon optimized artificial DNA sequence
catatgcatcaccaccaccaccacgctaaaaaattaccaaaagtaaaggggctggacaacaccgttgatatcattaaaggtggctacacctatgtgccgggcaagctggaggagttcgacagcaaagcgtttgaagtgcgtgcgctgggtggcaagaaaatcgcggttatgagcggcaaagaggcggcggaaattttctacgataacgagaagatggaacgtcagggcaccctgccgaaacgtatcgtgaacaccctgttcggcaagggtgcgattcacaccaccgcgggcaagaaacacgttgaccgtaaagcgctgtttatgagcctgatgaccgatgagaacctgaactacctgcgtgaactgacccgtaactattggttcatgaacaccgagcgtatgcaaagcatggacaaagtgaacgtttacaacgaaagcatctatatgctgaccaagattggctttcgttgggcgggtatcattcagaccccggaggaagcggagcaaaacgcgaaagacatggataccatgatcaacagcttcgtgagcctgggcagcgcgtacaagggttataagaaagcgaagaaagcgcgtaagcgtgtggaggactttctggaaaaacagatcattgatgttcgtaagggcaaactgcacccggaggaaggcaccgcgctgtacgagttcgcgcactgggaagacctgaacgataacccgatggacagccacctgtgcgcggttgatctgatgaacgtggttcgtccgctggcggcgatcaaccgtttcattagctatggtgtgaaagttctgattgaatttgaccaggagaaggaaaaactgcgtctggagaacaacgaagattacgcgtataagtttgcgcaagaggtgcgtcgtatcttcccgtttgttccgtacctgccgggtcgtgcggcggtggacctggaatacgatggctataaaattccggcgggtatgatgaccgcgctggacgtttatggcaccacccacgacgaggatctgtgggaaaacccggatcagttcaacccgaaccgttttgacaactgggatggcagcccgttcgacctgatcccgcaaggtggcggtgatttttacaccaaccaccgttgcgcgggcgagtggatcaccgtgatcattatggaggaaaccatgaagtacttcgcgaacaagatcgaatttgacgtgccgagccaagatctgagcgttaagctggacaaactgccgggcaacgttaccagcggcaccatcattagcaacgtgcgtccgcgtgttgcgcgtaagtaatgaaagctt

SEQ ID NO: 169 - DNA, 1329 bp, Artificial Sequence
  Codon optimized artificial DNA sequence
catatgcatcaccaccaccaccacgaaaacctgtacttccaaggcatggcgaagcgtatgccgaaagaccgtggtctggataacaccctgaccattatgaaagagggttacgaatatatccagaaccgtaccaagaaattcaacagcaacgtgtttgaaacccgtgtgctgggtggcaagcaagcggtggttattagcggcaaacaggcggcggaactgttttatgacaacgataaaattgagcgtagcggtaccctgccgccgcgtgtggttaagaccctgttcggcaaaggtgcgattcacaccaccaccggtaagaaacacatcgatcgtaaggcgctgtttatgagcctgatgaccgacgaaaacctgacctacctgcgtaaactgacccgtaactattggttccagaacacccaacgtatgcagctgatggatgaagtgaacgtttacaaggagagcatcattctgctgaccaaaattggctttcgttgggcgggtatcattgaaaccccggaaaacatcgagcaacacgcgatggacatggataagatgattgatagcttcggcgcgatcggtagcccgtacaaaggctatcgtgaagcgaagaaagcgcgtacccgtgttgaggactttctggaggaacaaatcattaagacccgtaagggcaaaattcatccggagccgggtagcgcgctgtacgaattcagccactgggaggacatgaacggtcagccgatggatagccgtctgtgcagcgtggacctgatgaacgttattcgtccgctggttgcgatcaaccgtttcgtggcgtatggcgttctggcgatgcacgaatttccgggcgagcgtacccgtgttgcgcgtgacgatgacgattacgcgtataagttcgtgcaagaagttcgtcgttactatccgttcgtgccgtttctgccgggcaaggcggcgaagaacatcgagttcgatggttacaagatcaagaaagacaccatgatcattctggatgtgttcggtaccctgcaccgtgaagacacctttagcgagccggaacgtttcaacccgtaccgttttgacaactgggatggcagcccgttcgatctgatcccgcagggtggcggtgactatcacaccaaccaccgttgcgcgggtgaatggatgaccatcattatcatggaggaaaccatgaagtacttcgcgcaagagattgactatgatgttccgccgcaggactttaccgtggatcgtaccaagctgccgggccgtgttaaaagcggtatgattatcaacaacgtgcgtctgaacatcgaccgtaccaaataactcgag

SEQ ID NO: 170 - DNA, 1329 bp, Artificial Sequence
  Codon optimized artificial DNA sequence
catatgcatcaccaccaccaccacgaaaacctgtacttccagggcatgagcaagccgattccggtggacaagggtatcgataaaaccctgaccgttatgaaagagggttacgaatatgtgagcaaccgtaccaagaaattcaacagcgatatttttgaaacccgtgttctgggtggcaagcgtgcgatggtgatcagcggcaaagaggcggcggaactgttctatgacaacgataagatcgagcgtaaaggtaccctgccgccgcgtgtggttaagaccctgtttggcaaaggtgcgattcacaccaccaccggcaaggttcacatcgaccgtaaaagcctgttcatgagcctgatgaccgaaaacaacctggagtacctgcgtaagctgacccgtaacttttggtatcagaacaccgaacgtatgcaactgatgggcaaggtgaacgtttacaaagagagcatcattctgctgaccaaagttggcttccgttgggcgggtgtgaccgagaagccggaaaaaattgagcagcacgcgatggacatggataagatgatcgatagcttcggcagcgtgggtaccgcgtacaaaggctatcgtgaagcgaagaaagcgcgtgcgcgtgttgaagactttctggagaagcagatcattgcgacccgtaagggcaaaattcacccggaggaaggtaccgcgctgcacagcttcagccactggcgtgactttcagggtaactacatggatagccgtctggcggcggtggacctgatgaacgttattcgtccgctggttgcgatcaaccgtttcgtggcgtatggcgttctggcgctgcacgagtttccgggcgagcgtgaacgtgtgagcgcggatgaaaacaactacgcgtataaatttacccaagagctgcgtcgttacttcccgtttgttccgttcctgccggcgaaggcgaaagttgacattgattttcagggccacgtgatcccgcaagacaccatgattgttctggatatcttcggtaccctgcaccgtgaagacctgtgggaagatccggagcgtttctacccggatcgttttgagaactgggacggcagcccgtttgatctgatcccgcagggtggcggtgactatcacaccaaccaccgttgcgcgggtgaatggatgaccatcattatcatggaggaaaccatgaagtacttcgcgcgtgagattacctatgacgttccggcgcaagactttaccgtggatcgtaccaagctgccgggccgtgtgaaaagcggtatggatatcgagaacgttcgtctgaacttcgaccgtaccctgtaactcgag

SEQ ID NO: 171 - DNA, 1323 bp, Artificial Sequence
  Codon optimized artificial DNA sequence
catatgcatcaccaccaccaccacgagaacctgtactttcagggcatgagcaccatcaagcgtgacaaaggtctggataacaccctgaaggtgatgaaagagggctacctgtataccaccaaccaacgtaaccgtctgggcgcggataacatctttgaaacccgtggtctgggtggcaagcgtgtgctggttctgagcggcaagaaagcggcggaaaccttctatgacaacgataagattgaacgtaaaggtaccctgccgaagcgtgtggttaacaccctgtttggcaaaggtgcgatccacaccaccaccggcaagaaacacattgatcgtaaggcgctgttcatgagcctgatgaccgagggtaacctgaaacacctgcgtgaactgacccgtaaccactggtacatgaacacccaccgtatggagcagatggaccaaatcaacatttatcgtgaaagcatcattctgctgaccaaagtgggcaccaaatgggcgggtgttcaggcgccggaggaaaaaatcgaggaaattgcgaccgacatggatatcatgattgacagctttcgtggcctgggtaccgcgttcaagggttaccgtagcagcgtgaaagcgcgtcgtcgtgttgaggactggctggaagatcagatcattcaaacccgtaagggcaaaatctttccgccggaaggtaccgcgctgtacgagttcgcgcactgggaagactatctgggcaacccgatggatagccgtctgtgcgcgattgacctgatgaacaccttccgtccgctgatcgcgattaaccgtttcgtgagctttggtctgctggcgatgcacgagtttccggttagccgtgagaagatcaacaacgaagacgattacgcgtatatgttcagccaagaagtgcgtcgtttctacccgtttgttccgttcctgccggcgaaggcgaaaaccgacttccactttgatggccacaaggtggacaaagataccatgctgctgctggatatctatggtaccatgcaccgtgaggacgtttttgaaaacccgaacgagtttcgtccggaacgtttcctggactgggatggtagcccgttcgatctgattccgcagggtggcggtgactactataccaaccaccgttgcgcgggcgagtggatgaccgtgatcattatggaggaaaccatgaaatactttgcgggtcgtatcacctatgatgtgccggaacaagacctgcgtgttgatctgaacagcatcccgggctacgttaagagcggtttcgtgattgagaacgttaaagaaaacctggaccgtcgttaactcgag

SEQ ID NO: 172 - DNA, 1323 bp, Artificial Sequence
  Codon optimized artificial DNA sequence
catatgcatcaccaccaccaccacgaaaacctgtacttccaaggcatgagcaccattaagaaagacaagggtctggataacaccctgaaggcgatcaaacagggctacaagtataccgaaaaccaacgtaaacgtctggacagcaacaacatttttgaaacccgtggtctgggtggtaaacgtatcattttcatcagcggtaaagaggcggcggaaaccttttatgacaacgataagatcgagcgtgagggtatgctgccgaaacgtgtggttagcaccctgttcggcaagggtgcgattcacaccaccaccggcaaggtgcacatcgatcgtaaagcgctgtttatgagcctgatgaccgagggtaacctgcagtacctgcgtgaactgacccgtaaccaatggcgtagcaacaccattaacatggaacgtcaggacaccgtgaacgtttatcgtgaggcgattgtgcaactgacccacatcggcacccgttgggcgggtgttcaggcgccggcggaggaaattgatcgtatcgcgaccgacatggatctgatgatcgacagcttcaaaggcctgggtctgagcttcaagggttttcgtgaaagcaaagcggcgcgtcgtcgtgtggaggactggctggaagatcagattcacaagacccgtaaaggcgttattcacccgccggagggtaccgcgctgtacgaattcgcgcactgggaggactttgaaggcaacccgatggatagccgtctgtgcgcgattgacctgatgaacaccttccgtccgctgattgcgatcaaccgtttcgtgacctttggtgttctggcgctgcaccaatatccggaaacccgtgagaagatcaaaaacgacccggattacgcgtatatgtttagccaggaagtgcgtcgttactatccgttcgttccgtttctgccggcgaaggcgaaagtggatttcgagtttgaaggccacaagattgacaaagatgtgatgatcctgctggatgtttacggtaccatgcaccgtgacgatgttttcgacaacccgaacgagttctatccggaacgttttaaggactgggatggcagcccgtttgatctgatcccgcaaggtggcggtgactaccacaccaaccaccgttgcgcgggtgaatggatgaccatcattatcatggaggaaaccatgaaatacttcgcggagcgtattacctatgacgtgccggaacaggacctgaccgttgatctggagaagctgccgggcagcgttgcgagcggtatgaacatcaccaacgtgcgtgagaacgttaaccgtaaataactcgag

SEQ ID NO: 173 - DNA, 1326 bp, Artificial Sequence
  Codon optimized artificial DNA sequence
catatgcatcaccaccaccaccacgagaacctgtacttccagggcatggcgaccatcaagcgtgataaaggtctggacaacagcgtgaaggttatgaaacagggctacctgtataccaccaaccaacgtgagcgtctgggcgtgaccgacggtgttttcgaaacccgtgcgctgggtggcaagcgtatcattgttctgagcggcaaagatggtgcggagctgttttatgataacgacaagattgaacgtagcggtaccctgccgaaacgtgtggttaacaccctgttcggcaagggtgcgatccacaccaccaccggcaaggtgcacattgaccgtaaagcgctgtttatgagcctgatgaccgagggtaacctgaaatacctgcgtgaactgacccgtaacctgtggtttgcgaacacccagcgtatggagagcatggatcaagtgaacgtttatcgtgaaagcatcattgtgctgaccaaagttggtacccgttgggcgggtgttcaagcgccggagaaggaaatcgagaacattgcgaccgatatggacatcatgattgacagcttcaaagcgatcggcagcgcgtttaagggttaccgtgaggcgaaagcggcgcgtcgtcgtgtggaagattggctggaggaccagatcattcaaacccgtaagggcaaaatccacccgccgaagggtaccgcgctgtacgagttcgcgcactggaaggattataaaggcgaaccgatggacagccgtctgtgcggtattgatctgatgaacacctttcgtccgctgatcgcgattaaccgtttcgttgcgtttggtgcgctggcgatgtacgagaacccggtggcgcgtgaaaagatcaaacaggacgatgactacgcgtatatgttcgcgcaagaagttcgtcgtttctacccgtttgtgccgtatctgccggcgaaggcgaaagttgactttcagtacaagggctatgaaatcgagaaagataccatgctggcgctggacatttacggtaccatgcacgatccgaacgtgtgggaagacccgaacgagttctatccggaacgttttaaggattgggatggcagcccgtttgacctgatcccgcaaggtggcggtgattaccacaccaaccaccgttgcgcgggcgagtggatgaccgttatcattatggaggaaaccatgaaatacttcgcgagccgtattaactatgacgttccggaacaggatctgaccgtggacctgaacagcctgccgggctatatcaagagcggttttgtgattgagaacgttcaagaaaacgtggatcgtacctaactcgag